(19) United States
(12) Patent Application Publication
(10) Pub. No.: US 2025/0223646 A1
(43) Pub. Date: Jul. 10, 2025

(54) METHODS FOR DETECTING AND TREATING CEREBRAL CAVERNOUS MALFORMATION

(71) Applicant: THE UNIVERSITY OF CHICAGO, Chicago, IL (US)

(72) Inventors: Issam AWAD, Chicago, IL (US); Romuald GIRARD, Chicago, IL (US)

(73) Assignee: THE UNIVERSITY OF CHICAGO, Chicago, IL (US)

(21) Appl. No.: 18/853,291

(22) PCT Filed: Mar. 30, 2023
(86) PCT No.: PCT/US2023/065144

§ 371 Date: Oct. 1, 2024

Related U.S. Application Data
(60) Provisional application No. 63/326,488, filed on Apr. 1, 2022.

(51) Int. Cl. C12Q 1/6883; C12N 15/115; G01N 33/68

(52) U.S. Cl. CPC C12Q 1/6883; C12N 15/115; G01N 33/6896; C12N 2310/16; C12N 2310/312; C12N 2320/32; C12Q 2600/158; C12Q 2600/178; G01N 2333/47; G01N 2333/54; G01N 2333/5428; G01N 2333/7151; G01N 2800/2871; G01N 2800/52; G01N 2800/60

(57) ABSTRACT

The current disclosure provides for methods for diagnosing, prognosing, and treating cerebral cavernous malformations (CCMs). Also provided are methods for determining the efficacy of therapeutic treatments for CCM, such as those that may be in a clinical trial setting or administered by a health care professional. Accordingly, aspects of the disclosure relate to method for treating cerebral cavernous malformations (CCMs) in a subject, the method comprising administering a therapy to a subject determined to have differential expression of one or more biomarkers selected from the biomarkers listed in Tables S1-S8 and S13 in a biological sample from the subject. Further aspects relate to A method for treating cerebral cavernous malformations (CCMs) in a subject, the method comprising administering a therapy to a subject determined to have differential expression of one or more biomarkers selected from let-7e-5p, miR-93-5p, miR-20b-5p, miR-128-3p, miR-9-5p, IL-10, Tsp-2, IL-2, and TNER1.

### CROSS-REFERENCE TO RELATED APPLICATIONS

This application claims the benefit of priority to U.S. Provisional Patent Application Ser. No. 63/326,488, filed Apr. 1, 2022, hereby incorporated by reference in its entirety.

### GOVERNMENT SUPPORT CLAUSE

This invention was made with government support under NS114552 awarded by the National Institutes of Health. The government has certain rights in the invention.

### I. FIELD OF THE INVENTION

The present invention relates generally to the fields of molecular biology and therapeutic diagnosis. More particularly, it concerns methods and compositions involving prognosing, diagnosing, and treating cerebral cavernous malformations.

### II. BACKGROUND

Cerebral cavernous malformations (CCMs), also known as cavernous angiomas, are enlarged blood-filled caverns prone to hemorrhage due to dysfunctional vessel wall angioarchitecture (1). A fraction of CCM patients manifest familial disease, with an autosomal dominant inheritance of a heterozygous germline loss of function mutation in one of three documented genes, CCM1/KRIT1, CCM2/Malcavernin, or CCM3/PDCD10 (1). These familial cases develop new hemorrhagic brain lesions throughout their life, predisposing them to a risk of symptomatic hemorrhage, seizures, and/or focal neurological deficits (1). The natural clinical course of individual lesions is relatively benign (2), but some cases acquire an aggressive course due to a number of mechanisms, including those related to the microbiome and gut-brain axis (3-5), lesional anticoagulant domains (6), and additional somatic mutations involving the activation of oncogenes (7). The CCM3/PDCD10 disease also manifests exceptional aggressiveness with greater lesion burden and earlier disease manifestations than other genotypes (8). Surgical excision of symptomatic CCM lesions is a current therapeutic option, but has serious morbidity and high costs, and obvious limitations with multiple lesions (1). Efforts are underway in the development of gene restoration therapies with viral vectors for familial disease (11).

There is an ongoing search for facile biomarkers that can accurately reflect disease status and lesional activity of CCMs, to guide the selection of aggressive cases for clinical trials and to monitor the impact of therapies (12-15). Thus, there is a need in the art for biomarkers for CCM that are indicative of disease level and progression.

### SUMMARY OF THE INVENTION

The current disclosure provides for methods for diagnosing, prognosing, and/or treating cerebral cavernous malformations (CCMs). Also provided are methods for determining the efficacy of therapeutic treatments for CCM, such as those that may be in a clinical trial setting or administered by a health care professional. Accordingly, the disclosure describes a method for treating cerebral cavernous malformations (CCMs) in a subject, the method comprising administering a therapy to a subject determined to have differential expression of one or more biomarkers selected from the biomarkers listed in Tables S1-S8 and S13 in a biological sample from the subject. Also described is a method for treating cerebral cavernous malformations (CCMs) in a subject, the method comprising administering a therapy to a subject determined to have differential expression of 1, 2, 3, 4, 5, 6, 7, 8, or 9 of the following biomarkers: let-7e-5p, miR-93-5p, miR-20b-5p, miR-128-3p, miR-9-5p, IL-10, Tsp-2, IL-2, and/or TNFR1. Also provided is a method for evaluating a subject comprising measuring the level of expression of one or more biomarkers listed in Tables S1-S8 and S13 in a biological sample from the subject. Also provided is a method for evaluating a subject comprising measuring the level of expression of 1, 2, 3, 4, 5, 6, 7, 8 or 9 of the following biomarkers: let-7e-5p, miR-93-5p, miR-20b-5p, miR-128-3p, miR-9-5p, IL-10, Tsp-2, IL-2, and TNER 1 in a sample from the subject.

The disclosure describes a method of prognosing, diagnosing, and/or determining the efficacy of a therapy in a subject, the method comprising: a) measuring the level of expression of one or more biomarkers listed in Tables S1-S8 and S13 in a sample from the subject; b) i) comparing the level(s) of expression of the one or more biomarkers to a control sample(s) or control level(s) of expression; or ii) calculating a canonical value; and, c) prognosing, diagnosing, and/or determining the efficacy of a therapy based on the levels of measured expression and/or the canonical value. Also described is a method of prognosing, diagnosing, and/or determining the efficacy of a therapy in a subject, the method comprising: a) measuring the level of expression of 1, 2, 3, 4, 5, 6, 7, 8, or 9 of the following biomarkers: let-7e-5p, miR-93-5p, miR-20b-5p, miR-128-3p, miR-9-5p, IL-10, Tsp-2, IL-2, and TNER1 in a sample from the subject; b) i) comparing the level(s) of expression of the one or more biomarkers to a control sample(s) or control level(s) of expression; and/or ii) calculating a canonical value; and, c) prognosing, diagnosing, and/or determining the efficacy of a therapy based on the levels of measured expression and/or the canonical value. The disclosure also relates to a kit comprising 1, 2, 3, 4, 5, 6, 7, 8, 9, 10, 11, 12, 13, 14, or 15 detection agents for determining expression levels of biomarkers for CCM, wherein the biomarkers comprise one or more listed in Tables S1-S8 and S13. Also described is a kit comprising 1, 2, 3, 4, 5, 6, 7, 8, 9, 10, 11, 12, 13, 14, or 15 detection agents for determining expression levels of biomarkers for CCM, wherein the biomarkers comprise 1, 2, 3, 4, 5, 6, 7, 8, or 9 of the following biomarkers: let-7e-5p, miR-93-5p, miR-20b-5p, miR-128-3p, miR-9-5p, IL-10, Tsp-2, IL-2, and TNER1.

The method may comprise measuring, comprise measuring at most, or comprise measuring at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 biomarkers (or any derivable range therein) listed in Tables S1-S8. The method may exclude the measurement or evaluation of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 specific biomarkers (or any derivable range therein) listed in Tables S1-S8. The methods may also exclude the measurement, evaluation, or determination of a CCM biomarker of Tables S1-S8 and S13. The methods may exclude the measurement, evaluation, or determination of 1, 2, 3, 4, 5, 6, 7, 8, or 9 of the following biomarkers: let-7e-5p, miR-93-5p, miR-20b-5p, miR-128-3p, miR-9-5p, IL-10, Tsp-2, IL-2, and TNER1. The subject may be one in which the expression level of 1, 2, 3, 4, 5, 6, 7, 8, or 9 of the following biomarkers: let-7e-5p, miR-93-5p, miR-20b-5p, miR-128-3p, miR-9-5p, IL-10, Tsp-2, IL-2, and TNER1 has not been detected, evaluated, measured, or determined. Also described is a method for treating cerebral cavernous malformations (CCMs) in a subject, the method comprising administering one or more miRNA modulators, wherein the miRNA comprises let-7e-5p, miR-93-5p, miR-20b-5p, miR-128-3p, and/or miR-9-5p.

The subject may be one that has been determined to have to have differential expression of 1, 2, 3, 4, 5, or 6 of the following biomarkers: let-7e-5p, miR-93-5p, miR-20b-5p, miR-128-3p, IL-10, and Tsp-2. The expression levels of 1, 2, 3, 4, 5, or 6 of the following biomarkers: let-7e-5p, miR-93-5p, miR-20b-5p, miR-128-3p, IL-10, and Tsp-2 may be evaluated, measured, or determined in the sample. The subject may be one that has been determined to have differential expression of let-7e-5p, miR-93-5p, miR-20b-5p, miR-128-3p, IL-10, and Tsp-2. The expression levels of let-7e-5p, miR-93-5p, miR-20b-5p, miR-128-3p, IL-10, and Tsp-2 may be evaluated, measured, or determined in the sample. The methods may exclude the measurement, evaluation, or determination of 1, 2, 3, 4, 5, or 6 of the following biomarkers: let-7e-5p, miR-93-5p, miR-20b-5p, miR-128-3p, IL-10, and Tsp-2. The subject may be one in which the expression level of 1, 2, 3, 4, 5, or 6 of the following biomarkers: let-7e-5p, miR-93-5p, miR-20b-5p, miR-128-3p, IL-10, and Tsp-2 has not been detected, evaluated, measured, or determined. The methods may comprise or further comprise determining a canonical value. The canonical value is or may be used to determine a cut-off value for diagnosing, prognosing, and/or predicting efficacy to a therapeutic treatment. The subject may be one that has been determined to have a canonical value of greater than 0, wherein the canonical value is equal to −114.9*[let-7e-5p]ᵣ+359.5*[miR-93-5p]ᵣ−118.2*[miR-20b-5p]ᵣ+88.9*[miR-128-3p]ᵣ+199.5*[IL-10]−19.7*[Tsp-2]−463.2, wherein [X]ᵣ denotes the relative quantification value for each miRNA biomarker. The subject may be one that has been determined to have a canonical value equal to, greater than, less than, at most, or at least −300, −299, −298, −297, −296, −295, −294, −293, −292, −291, −290, −289, −288, −287, −286, −285, −284, −283, −282, −281, −280, −279, −278, −277, −276, −275, −274, −273, −272, −271, −270, −269, −268, −267, −266, −265, −264, −263, −262, −261, −260, −259, −258, −257, −256, −255, −254, −253, −252, −251, −250, −249, −248, −247, −246, −245, −244, −243, −242, −241, −240, −239, −238, −237, −236, −235, −234, −233, −232, −231, −230, −229, −228, −227, −226, −225, −224, −223, −222, −221, −220, −219, −218, −217, −216, −215, −214, −213, −212, −211, −210, −209, −208, −207, −206, −205, −204, −203, −202, −201, −200, −199, −198, −197, −196, −195, −194, −193, −192, −191, −190, −189, −188, −187, −186, −185, −184, −183, −182, −181, −180, −179, −178, −177, −176, −175, −174, −173, −172, −171, −170, −169, −168, −167, −166, −165, −164, −163, −162, −161, −160, −159, −158, −157, −156, −155, −154, −153, −152, −151, −150, −149, −148, −147, −146, −145, −144, −143, −142, −141, −140, −139, −138, −137, −136, −135, −134, −133, −132, −131, −130, −129, −128, −127, −126, −125, −124, −123, −122, −121, −120, −119, −118, −117, −116, −115, −114, −113, −112, −111, −110, −109, −108, −107, −106, −105, −104, −103, −102, −101, −100, −99, −98, −97, −96, −95, −94, −93, −92, −91, −90, −89, −88, −87, −86, −85, −84, −83, −82, −81, −80, −79, −78, −77, −76, −75, −74, −73, −72, −71, −70, −69, −68, −67, −66, −65, −64, −63, −62, −61, −60, −59, −58, −57, −56, −55, −54, −53, −52, −51, −50, −49, −48, −47, −46, −45, −44, −43, −42, −41, −40, −39, −38, −37, −36, −35, −34, −33, −32, −31, −30, −29, −28, −27, −26, −25, −24, −23, −22, −21, −20, −19, −18, −17, −16, −15, −14, −13, −12, −11, −10, −9, −8, −7, −6, −5, −4, −3, −2, −1, 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700 or any derivable range therein.

The subject may be diagnosed as having CCM, prognosed as being high risk, or the efficacy of the therapy is determined to be ineffective when the canonical value is greater than 0. The subject may be diagnosed as having CCM, prognosed as being high risk, or the efficacy of the therapy is determined to be ineffective when the canonical value is greater than, is exactly, or is at least −20, −19, −18, −17, −16, −15, −14, −13, −12, −11, −10, −9, −8, −7, −6, −5, −4, −3, −2, −1, 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, or any derivable range therein.

The subject may be diagnosed as not having CCM, prognosed as low risk, and/or the efficacy of the therapy is determined to be effective when the canonical value is less than 300. The subject may be diagnosed as not having CCM, prognosed as low risk, and/or the efficacy of the therapy is determined to be effective when the canonical value is lower than, is exactly, or is at most −300, −299, −298, −297, −296, −295, −294, −293, −292, −291, −290, −289, −288, −287, −286, −285, −284, −283, −282, −281, −280, −279, −278, −277, −276, −275, −274, −273, −272, −271, −270, −269, −268, −267, −266, −265, −264, −263, −262, −261, −260, −259, −258, −257, −256, −255, −254, −253, −252, −251, −250, −249, −248, −247, −246, −245, −244, −243, −242, −241, −240, −239, −238, −237, −236, −235, −234, −233, −232, −231, −230, −229, −228, −227, −226, −225, −224, −223, −222, −221, −220, −219, −218, −217, −216, −215, −214, −213, −212, −211, −210, −209, −208, −207, −206, −205, −204, −203, −202, −201, −200, −199, −198, −197, −196, −195, −194, −193, −192, −191, −190, −189, −188, −187, −186, −185, −184, −183, −182, −181, −180, −179, −178, −177, −176, −175, −174, −173, −172, −171, −170, −169, −168, −167, −166, −165, −164, −163, −162, −161, −160, −159, −158, −157, −156, −155, −154, −153, −152, −151, −150, −149, −148, −147, −146, −145, −144, −143, −142, −141, −140, −139, −138, −137, −136, −135, −134, −133, −132, −131, −130, −129, −128, −127, −126, −125, −124, −123, −122, −121, −120, −119, −118, −117, −116, −115, −114, −113, −112, −111, −110, −109, −108, −107, −106, −105, −104, −103, −102, −101, −100, −99, −98, −97, −96, −95, −94, −93, −92, −91, −90, −89, −88, −87, −86, −85, −84, −83, −82, −81, −80, −79, −78, −77, −76, −75, −74, −73, −72, −71, −70, −69, −68, −67, −66, −65, −64, −63, −62, −61, −60, −59, −58, −57, −56, −55, −54, −53, −52, −51, −50, −49, −48, −47, −46, −45, −44, −43, −42, −41, −40, −39, −38, −37, −36, −35, −34, −33, −32, −31, −30, −29, −28, −27, −26, −25, −24, −23, −22, −21, −20, −19, −18, −17, −16, −15, −14, −13, −12, −11, −10, −9, −8, −7, −6, −5, −4, −3, −2, −1, 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300 or any derivable range therein.

The subject may be one that has been determined to have to have differential expression of 1, 2, 3, 4, or 5 of the following biomarkers: miR-9-5p, miR-93-5p, IL-2, TNFR1, and Tsp-2. The expression levels of 1, 2, 3, 4, or 5 of the following biomarkers: miR-9-5p, miR-93-5p, IL-2, TNFR1, and Tsp-2 may be evaluated, measured, or determined in the sample. The subject may be one that has been determined to have to have differential expression of miR-9-5p, miR-93-5p, IL-2, TNFR1, and Tsp-2. The expression levels of miR-9-5p, miR-93-5p, IL-2, TNFR1, and Tsp-2 may be evaluated, measured, or determined in the sample. The methods may exclude the measurement, evaluation, or determination of 1, 2, 3, 4, or 5 of the following biomarkers: miR-9-5p, miR-93-5p, IL-2, TNFR1, and Tsp-2. The subject may be one in which the expression level of 1, 2, 3, 4, or 5 of the following biomarkers: miR-9-5p, miR-93-5p, IL-2, TNFR1, and Tsp-2 has not been detected, evaluated, measured, or determined. The methods may comprise or further comprise determining a canonical value. The canonical value is or may be used to determine a cut-off value for diagnosing, prognosing, and/or predicting efficacy to a therapeutic treatment. The subject may be one that has been determined to have a canonical value of less than 15, wherein the canonical value is equal to 2.1*[miR-9-5p]ᵣ−3.0*[miR-93-5p], −0.01*[IL-2]+0.1*[TNFR1]+0.5*[Tsp-2], wherein [X]ᵣ denotes the relative quantification value for each miRNA biomarker. The subject may be one that has been determined to have a canonical value equal to, greater than, less than, at most, or at least −10, −9.8, −9.6, −9.4, −9.2, −9, −8.8, −8.6, −8.4, −8.2, −8, −7.8, −7.6, −7.4, −7.2, −7, −6.8, −6.6, −6.4, −6.2, −6, −5.8, −5.6, −5.4, −5.2, −5, −4.8, −4.6, −4.4, −4.2, −4, −3.8, −3.6, −3.4, −3.2, −3, −2.8, −2.6, −2.4, −2.2, −2, −1.8, −1.6, −1.4, −1.2, −1, −0.8, −0.6, −0.4, −0.2, 0, 0.2, 0.4, 0.6, 0.8, 1, 1.2, 1.4, 1.6, 1.8, 2, 2.2, 2.4, 2.6, 2.8, 3, 3.2, 3.4, 3.6, 3.8, 4, 4.2, 4.4, 4.6, 4.8, 5, 5.2, 5.4, 5.6, 5.8, 6, 6.2, 6.4, 6.6, 6.8, 7, 7.2, 7.4, 7.6, 7.8, 8, 8.2, 8.4, 8.6, 8.8, 9, 9.2, 9.4, 9.6, 9.8, 10, 10.2, 10.4, 10.6, 10.8, 11, 11.2, 11.4, 11.6, 11.8, 12, 12.2, 12.4, 12.6, 12.8, 13, 13.2, 13.4, 13.6, 13.8, 14, 14.2, 14.4, 14.6, 14.8, 15, 15.2, 15.4, 15.6, 15.8, 16, 16.2, 16.4, 16.6, 16.8, 17, 17.2, 17.4, 17.6, 17.8, 18, 18.2, 18.4, 18.6, 18.8, 19, 19.2, 19.4, 19.6, 19.8, 20, 20.2, 20.4, 20.6, 20.8, 21, 21.2, 21.4, 21.6, 21.8, 22, 22.2, 22.4, 22.6, 22.8, 23, 23.2, 23.4, 23.6, 23.8, 24, 24.2, 24.4, 24.6, 24.8, 25, 25.2, 25.4, 25.6, 25.8, 26, 26.2, 26.4, 26.6, 26.8, 27, 27.2, 27.4, 27.6, 27.8, 28, 28.2, 28.4, 28.6, 28.8, 29, 29.2, 29.4, 29.6, 29.8, 30, 30.2, 30.4, 30.6, 30.8, 31, 31.2, 31.4, 31.6, 31.8, 32, 32.2, 32.4, 32.6, 32.8, 33, 33.2, 33.4, 33.6, 33.8, 34, 34.2, 34.4, 34.6, 34.8, 35, or any range derivable therein.

The subject may be diagnosed as having CCM, prognosed as being high risk, or the efficacy of the therapy is determined to be ineffective when the canonical value is less than 15. The subject may be diagnosed as having CCM, prognosed as being high risk, or the efficacy of the therapy is determined to be ineffective when the canonical value is less than, is exactly, or is at most 0, 0.2, 0.4, 0.6, 0.8, 1, 1.2, 1.4, 1.6, 1.8, 2, 2.2, 2.4, 2.6, 2.8, 3, 3.2, 3.4, 3.6, 3.8, 4, 4.2, 4.4, 4.6, 4.8, 5, 5.2, 5.4, 5.6, 5.8, 6, 6.2, 6.4, 6.6, 6.8, 7, 7.2, 7.4, 7.6, 7.8, 8, 8.2, 8.4, 8.6, 8.8, 9, 9.2, 9.4, 9.6, 9.8, 10, 10.2, 10.4, 10.6, 10.8, 11, 11.2, 11.4, 11.6, 11.8, 12, 12.2, 12.4, 12.6, 12.8, 13, 13.2, 13.4, 13.6, 13.8, 14, 14.2, 14.4, 14.6, 14.8, 15, or any derivable range therein.

The subject may be diagnosed as not having CCM, prognosed as low risk, and/or the efficacy of the therapy is determined to be effective when the canonical value is greater than 10. The subject may be diagnosed as not having CCM, prognosed as low risk, and/or the efficacy of the therapy is determined to be effective when the canonical value is greater than, is exactly, or is at least 8, 8.2, 8.4, 8.6, 8.8, 9, 9.2, 9.4, 9.6, 9.8, 10, 10.2, 10.4, 10.6, 10.8, 11, 11.2, 11.4, 11.6, 11.8, 12, 12.2, 12.4, 12.6, 12.8, 13, 13.2, 13.4, 13.6, 13.8, 14, 14.2, 14.4, 14.6, 14.8, 15, 15.2, 15.4, 15.6, 15.8, 16, 16.2, 16.4, 16.6, 16.8, 17, 17.2, 17.4, 17.6, 17.8, 18, 18.2, 18.4, 18.6, 18.8, 19, 19.2, 19.4, 19.6, 19.8, 20, 20.2, 20.4, 20.6, 20.8, 21, 21.2, 21.4, 21.6, 21.8, 22, 22.2, 22.4, 22.6, 22.8, 23, 23.2, 23.4, 23.6, 23.8, 24, 24.2, 24.4, 24.6, 24.8, 25, 25.2, 25.4, 25.6, 25.8, 26, 26.2, 26.4, 26.6, 26.8, 27, 27.2, 27.4, 27.6, 27.8, 28, 28.2, 28.4, 28.6, 28.8, 29, 29.2, 29.4, 29.6, 29.8, 30, or any derivable range therein.

Relative and absolute quantifications of miRNAs can be assessed using RT-qPCR, for example. An exogenous spike-in control miRNA can be added to samples prior to extraction and then measured to correct for extraction efficiency. The absolute quantification (number of miRNAs strands/μl) of each miRNA can be estimated using a standard curve comprised of serial dilutions of known concentrations of the miRNAs of interest. For the relative quantification, an endogenous miRNA control can be added during the extraction and used as it is expected to be expressed at equal levels across tissue types and samples. The comparison of the relative quantification of each pre-defined miRNA can be performed between (1) familial-CCM1 patients, (2) familial-CCM3 patients, and (3) healthy non-CCM controls. A relative quantification value that is greater than +4, 3, 2, or 1 standard deviations away from the mean may be defined as an outlier. Plasma proteins can be measured using the enzyme-linked immunosorbent assay (ng/ml) and Multiplex (pg/ml) assay in accordance with manufacturer protocols. The plasma levels of thrombomodulin (TM), thrombospondin-1 (Tsp-1), thrombospondin-2 (Tsp-2) can be assessed using commercially available ELISA assays kits (R&D Systems, Minneapolis, Minnesota, USA). The plasma levels of other CCM biomarkers, such as, tumor necrosis factor alpha (TNFα), tumor necrosis factor receptor 1 (TNFR1), matrix metalloproteinase-2 (MMP2) and −9 (MMP9), chemokine ligand 2 (CCL2/MCP1), soluble endoglin/CD105 (sENG), soluble vascular cell adhesion protein 1 (sVCAM1), soluble intercellular adhesion molecule 1 (sICAM1/CD54), interleukin-1 beta (IL-1B), IL-2, IL-6, IL-8/CXCL-8, IL-10, vascular endothelial growth factor (VEGF), soluble roundabout guidance receptor 4 (sROBO4), interferon gamma (IFNγ), soluble cluster of differentiation 14 (sCD14), and C-reactive protein (CRP) can be assessed using customized magnetic bead-based multiplex Luminex screening immunoassay kits (R&D Systems, Minneapolis, Minnesota, USA), for example. The measurements can be performed with a BioRad BioPlex-100 analyzer (Bio-Rad Laboratories, Hercules, California, USA) running the BioPlex Manager Software version 5.0, or the Luminex 200 System (Luminex Corporation, Austin, Texas, USA) running with xPONENT Software. Plasma values greater±4, 3, 2, or 1 standard deviations from the mean may be excluded as outliers. Batch effects can be corrected using the Combat function from the R package SVA (14). Differences in relative and absolute levels of miRNAs measured by RT-qPCR, as well as biomarker canonical values between genotypes and non-CCM healthy controls can be analyzed using an unpaired two samples Student's t-test, assessed with pooled standard deviation, or Mann-Whitney test according to the equality of the variance. Canonical values of the best weighted combinations developed during the three-step Bayesian integrative approach can be calculated for each subject and compared between familial-CCM patients and healthy-non-CCM subjects using an unpaired two sample Student's t-test, assessed with pooled standard deviation, or Mann-Whitney test according to the equality of the variance. The expression level used to determine a canonical value may be an absolute, relative, normalized, or corrected value.

The biomarker may be increased or decreased relative to a control. The subject may be one in which the sample from the subject has been determined to a have a biomarker increased relative to a control. The subject may be one in which the sample from the subject has been determined to a have a biomarker decreased relative to a control. The biomarker may be determined, evaluated, or measured as one that is increased relative to a control. The biomarker may be determined, evaluated, or measured as one that is decreased relative to a control. The control may comprise the expression or protein level of the biomarker in samples from subjects identified as low risk or in subjects identified as not having CCM. The control may comprise the expression or protein level of the biomarker in samples from subjects identified as high risk or in subjects identified as having CCM. The subject may be one that has been diagnosed with CCM. The subject may be one that has not been diagnosed with CCM. The CCM may be further defined as familial CCM. The familial CCM may be further defined as familial-CCM1. Familial-CCM1 refers to CCM in a subject having a mutation in the CCM1 gene. The familial CCM may be further defined as familial-CCM2. Familial-CCM2 refers to CCM in a subject having a mutation in the CCM2 gene. The familial CCM may be further defined as familial-CCM3. Familial-CCM3 refers to CCM in a subject having a mutation in the CCM3 gene. The subject may be one that is being treated with a therapy for CCM. The subject may be one that is undergoing a clinical trial for a CCM therapy. The subject may be one that is in need of monitoring the effectiveness of a CCM therapy. The subject may be one that is being treated with a gene therapy for CCM. Other CCM therapies include, for example, surgical excision of a CCM lesion and B-cell immunomodulation therapy. Gene restoration therapy is known and contemplated in the art and may comprise using an endothelial specific viral vectors. These aspects are further described in Albright, B. H., et al., Mol Ther. 2018 Feb. 7;26 (2): 510-523, Tse L.V. et al., Proc Natl Acad Sci USA. 2017 Jun. 13;114 (24): E4812-E4821, and de Leeuw C.N. et al., Mol Brain. 2016 May 10;9 (1): 52, each of which are incorporated by reference in their entirety.

The sample from the subject may comprise a tissue sample, a blood sample, a whole blood sample, a fractionated sample, a plasma sample, a fecal sample, or a urine sample. The sample from the subject may comprise a plasma sample from the subject. The sample may also be a sample described herein.

At least the expression level of miR-9-5p was, may be, or is determined, measured, or evaluated in the sample. At least the expression level of miR-93-5p was, can be, or is determined, measured, or evaluated in the sample. At least the expression level of IL-2 was, can be, or is determined, measured, or evaluated in the sample. At least the expression level of TNFR1 was, can be, or is determined, measured, or evaluated in the sample. At least the expression level of Tsp-2 was, can be, or is determined, measured, or evaluated in the sample. At least the expression level of let-7e-5p was, can be, or is determined, measured, or evaluated in the sample. At least the expression level of miR-20b-5p was, can be, or is determined, measured, or evaluated in the sample. At least the expression level of miR-128-3p was, can be, or is determined, measured, or evaluated in the sample. At least the expression level of IL-10 was, can be, or is determined, measured, or evaluated in the sample. The methods may exclude the determination, evaluation, or measurement of the expression level of a biomarker or a CCM biomarker in the sample, other than those specifically recited.

The expression levels of the biomarkers may be determined by measuring the protein levels or the RNA levels of the biomarker. The expression level(s) of the biomarker(s) can be determined by evaluating the RNA level of the biomarker. The expression level(s) of the biomarker(s) may be or have been determined by evaluating the protein level of the biomarker. The determined, measured, or evaluated biomarker may comprise 1, 2, 3, 4, or 5 of the following biomarkers: let-7e-5p, miR-93-5p, miR-20b-5p, miR-128-3p, and miR-9-5p, and wherein the expression level(s) of the biomarker(s) were determined by evaluating or measuring the RNA level of the biomarker. The determined, measured, or evaluated biomarker may comprise 1, 2, 3, or 4 of the following biomarkers: IL-10, Tsp-2, IL-2, and TNER1, and wherein the expression level(s) of the biomarker(s) were determined by evaluating or measuring the protein level of the biomarker.

Methods of the disclosure may comprise or further comprise treating a subject with a CCM therapy. The subject being treated may be one that has been diagnosed as having CCM, prognosed as high risk, and/or determined to have an ineffective therapeutic response. The therapy may comprise or further comprise a gene therapy, a B-cell immunomodulation therapy, surgical excision of a CCM lesion, or combinations thereof. The methods may also exclude a certain CCM therapy. Accordingly, methods may exclude a B-cell immunomodulation therapy, surgical excision of a CCM lesion, or combinations thereof.

The modulator of the methods may be an agomer of a miRNA biomarker described herein. The modulator may be an antagomir of the miRNA biomarker described herein.

As used herein, the term “agomir” refers to a synthetic oligonucleotide or oligonucleotide mimetic that functionally mimics a miRNA. An agomir can be an oligonucleotide with the same or similar nucleic acid sequence to a miRNA or a portion of a miRNA. The agomir may have 1, 2, 3, 4, 5, 6, 7, 8, 9 or 10 nucleotide differences from the miRNA that it mimics. Further, agomirs can have the same length, a longer length or a shorter length than the miRNA that it mimics. The agomir may have the same sequence as 6-8 nucleotides at the 5′ end of the miRNA it mimics. An agomir can be 5, 6, 7, 8, 9, 10, 11, 12, 13, 14, 15, 16, 17, 18, 19, 20, 21, 22, 23, 24, 25, 27, 28, 29, 30, 31, 32, 33, 34, 35, 36, 37, 38, 39, 40, 41, 42, 43, 44, 45, 46, 47, 48, 49 or 50 nucleotides in length. An agomir can be 5-10, 6-8, 10-20, 10-15 or 5-500 nucleotides in length. Agomirs include any of the sequences shown in Tables 1, 11, 13 and 14. These chemically modified synthetic RNA duplexes include a guide strand that is identical or substantially identical to the miRNA of interest to allow efficient loading into the miRISC complex, whereas the passenger strand is chemically modified to prevent its loading to the Argonaute protein in the miRISC complex (Thorsen S B et al., Cancer J., 18 (3): 275-284 (2012); Broderick J A et al., Gene Ther., 18 (12): 1104-1110 (2011)).

As used herein, the term “antagomir” refers to a synthetic oligonucleotide or oligonucleotide mimetic having complementarity to a specific microRNA, and which inhibits the activity of that miRNA. The antagomir may have 1, 2, 3, 4, 5, 6, 7, 8, 9 or 10 nucleotide differences from the miRNA that it inhibits. Further, antagomirs can have the same length, a longer length or a shorter length than the miRNA that it inhibits. The antagomir may hybridize to 6-8 nucleotides at the 5′ end of the miRNA it inhibits. An antagomir can be 5, 6, 7, 8, 9, 10, 11, 12, 13, 14, 15, 16, 17, 18, 19, 20, 21, 22, 23, 24, 25, 27, 28, 29, 30, 31, 32, 33, 34, 35, 36, 37, 38, 39, 40, 41, 42, 43, 44, 45, 46, 47, 48, 49 or 50 nucleotides in length. An antagomir can be 5-10, 6-8, 10-20, 10-15 or 5-500 nucleotides in length. Antagomirs can include nucleotides that are complementary to any of the sequences shown in Tables 1, 11, 13 and 14. The antagomirs are synthetic reverse complements that tightly bind to and inactivate a specific miRNA. Various chemical modifications are used to improve nuclease resistance and binding affinity. The most commonly used modifications to increase potency include various 2′ sugar modifications, such as 2′-O-Me, 2′-O-methoxyethyl(2′-MOE), or 2′-fluoro (2′-F). The nucleic acid structure of the miRNA can also be modified into a locked nucleic acid (LNA) with a methylene bridge between the 2′ oxygen and the 4′ carbon to lock the ribose in the 3′-endo (North) conformation in the A-type conformation of nucleic acids (Lennox K A et al., Gene Ther. December 2011; 18 (12): 1111-1120; Bader A G et al. Gene Ther. December 2011; 18 (12): 1121-1126).

The modulator may be linked to a targeting moiety. The targeting moiety may be an aptamir. As used herein, the term “aptamir” refers to the combination of an aptamer (oligonucleic acid or peptide molecule that bind to a specific target molecule) and an agomir or antagomir as defined above, which allows cell or tissue-specific delivery of the miRNA agents. The targeting moiety may be one that delivers the modulator to a specific cell type or tissue. The modulator may comprise a modified oligonucleotide. The modified oligonucleotide may comprise a locked nucleotide, ethylene bridged nucleotide, a peptide nucleic acid, or a 5′ (E)-vinyl-phosphonate (VP) modification. The modification may also be one described herein. The modulator may be administered by systemic, intravenous, intramuscular, or parenteral administration. The route of administration may also be one described herein.

Kits of the disclosure may comprise one or more negative or positive control samples and/or control detection agents. The kit may comprise detection agents for CCM biomarkers, wherein the CCM biomarkers comprise 1, 2, 3, 4, 5, or 6 of the following biomarkers: let-7e-5p, miR-93-5p, miR-20b-5p, miR-128-3p, IL-10, and Tsp-2. The kit may comprise detection agents for CCM biomarkers, wherein the CCM biomarkers exclude 1, 2, 3, 4, 5, or 6 of the following biomarkers: let-7e-5p, miR-93-5p, miR-20b-5p, miR-128-3p, IL-10, and Tsp-2. The kit may comprise detection agents for CCM biomarkers, wherein the CCM biomarkers consist of let-7e-5p, miR-93-5p, miR-20b-5p, miR-128-3p, IL-10, and Tsp-2. The kit may comprise reagents for detecting CCM biomarkers, wherein the CCM biomarkers comprise 1, 2, 3, 4, or 5 of the following biomarkers: miR-9-5p, miR-93-5p, IL-2, TNFR1, and Tsp-2. The kit may comprise reagents for detecting CCM biomarkers, wherein the CCM biomarkers exclude 1, 2, 3, 4, or 5 of the following biomarkers: miR-9-5p, miR-93-5p, IL-2, TNFR1, and Tsp-2. The kit may comprise reagents for detecting CCM biomarkers, wherein the CCM biomarkers consist of miR-9-5p, miR-93-5p, IL-2, TNFR1, and Tsp-2. The kit may further comprise one or more negative or positive control samples and/or control detection agents. The kit may further comprise instructions for use. The kit may comprise or further comprise instructions for use. The CCM biomarkers may comprise or consist of let-7e-5p, miR-93-5p, miR-20b-5p, miR-128-3p, IL-10, Tsp-2 and/or one or more controls. The instructions may comprise evaluating a canonical value, wherein the canonical value is equal to −114.9*[let-7e-5p]ᵣ+359.5*[miR-93-5p]ᵣ−118.2*[miR-20b-5p]ᵣ+88.9*[miR-128-3p]ᵣ+199.5*[IL-10]−19.7*[Tsp-2]−463.2, wherein [X]ᵣ denotes the relative quantification value for each miRNA biomarker. The CCM biomarkers may comprise or consist of miR-9-5p, miR-93-5p, IL-2, TNFR1, Tsp-2.and/or one or more controls. The instructions may comprise evaluating a canonical value, wherein the canonical value is equal to 2.1*[miR-9-5p]ᵣ−3.0*[miR-93-5p]ᵣ−0.01*[IL-2]+0.1*[TNFR1]+0.5*[Tsp-2], wherein [X]ᵣ denotes the relative quantification value for each miRNA biomarker. The kit may comprise reagents for isolation and/or amplification of biomarkers from a biological sample.

The subject may be a mammal. The subject may comprise a laboratory test animal, such as a mouse, rat, rabbit, dog, cat, horse, or pig. The subject may be a human.

“Treatment” or treating may refer to any treatment of a disease in a mammal, including: (i) preventing the disease, that is, causing the clinical symptoms of the disease not to develop by administration of a protective composition prior to the induction of the disease; (ii) suppressing the disease, that is, causing the clinical symptoms of the disease not to develop by administration of a protective composition after the inductive event but prior to the clinical appearance or reappearance of the disease; (iii) inhibiting the disease, that is, arresting the development of clinical symptoms by administration of a protective composition after their initial appearance; and/or (iv) relieving the disease, that is, causing the regression of clinical symptoms by administration of a protective composition after their initial appearance. The treatment may exclude prevention of the disease.

Throughout this application, the term “about” is used according to its plain and ordinary meaning in the area of cell and molecular biology to indicate that a value includes the standard deviation of error for the device or method being employed to determine the value.

The use of the word “a” or “an” when used in conjunction with the term “comprising” may mean “one,” but it is also consistent with the meaning of “one or more,” “at least one,” and “one or more than one.”

As used herein, the terms “or” and “and/or” are utilized to describe multiple components in combination or exclusive of one another. For example, “x, y, and/or z” can refer to “x” alone, “y” alone, “z” alone, “x, y, and z,” “(x and y) or z,” “x or (y and z),” or “x or y or z.” It is specifically contemplated that x, y, or z may be specifically excluded from an embodiment or aspect.

The words “comprising” (and any form of comprising, such as “comprise” and “comprises”), “having” (and any form of having, such as “have” and “has”), “including” (and any form of including, such as “includes” and “include”), “characterized by” (and any form of including, such as “characterized as”), or “containing” (and any form of containing, such as “contains” and “contain”) are inclusive or open-ended and do not exclude additional, unrecited elements or method steps.

The compositions and methods for their use can “comprise,” “consist essentially of,” or “consist of” any of the ingredients or steps disclosed throughout the specification. The phrase “consisting of” excludes any element, step, or ingredient not specified. The phrase “consisting essentially of” limits the scope of described subject matter to the specified materials or steps and those that do not materially affect its basic and novel characteristics. It is contemplated that embodiments and aspects described in the context of the term “comprising” may also be implemented in the context of the term “consisting of” or “consisting essentially of.”

Any method in the context of a therapeutic, diagnostic, or physiologic purpose or effect may also be described in “use” claim language such as “Use of” any compound, composition, or agent discussed herein for achieving or implementing a described therapeutic, diagnostic, or physiologic purpose or effect.

Use of the one or more sequences or compositions may be employed based on any of the methods described herein. Other embodiments are discussed throughout this application. Any embodiment or aspect discussed with respect to one aspect of the disclosure applies to other aspects of the disclosure as well and vice versa.

It is specifically contemplated that any limitation discussed with respect to one embodiment or aspect of the invention may apply to any other embodiment or aspect of the invention. Furthermore, any composition of the invention may be used in any method of the invention, and any method of the invention may be used to produce or to utilize any composition of the invention. Aspects of an embodiment set forth in the Examples are also embodiments that may be implemented in the context of embodiments discussed elsewhere in a different Example or elsewhere in the application, such as in the Summary of Invention, Detailed Description of the Embodiments, Claims, and description of Figure Legends.

Other objects, features and advantages of the present invention will become apparent from the following detailed description. It should be understood, however, that the detailed description and the specific examples, while indicating specific embodiments and aspects of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1. Methodological Overview for Identification of Mechanistically Relevant Homologous Differentially Expressed (DE) miRNAs. A parallel methodological pipeline was applied for murine models and human subjects to identify DE miRNA in the plasma of CCM1 and CCM3 genotypes compared to healthy controls, wild type, and each other. Once homologous DE miRNA were identified using the MirGeneDB database, MirWalk 3.0 was used to query putative gene targets of each homologous DE miRNA in mice and then in patients. Gene targets were compared to DE genes within previously published CCM transcriptomes, including the transcriptomes of mouse in vitro Ccm1^{-/-} BMECs, and Ccm3^{-/-} brain microvascular endothelial cells (BMECs), as well as in vivo Ccm3^{+/-} lesional neurovascular units (NVUs) and laser micro-dissected NVUs of human surgically resected CCM lesions. Illustrative images represent microCT of Ccm3^{-/-} mouse brain, and SWI (susceptibility weighted imaging) MRI of human brain with CCM disease and CCM3 genotype.

FIG. 2. Homologous Differentially Expressed (DE) Plasma miRNAs in Mouse and Human CCM1 and CCM3 Genotypes. This was constructed to portray commonalities in DE miRNAs between human and mice in each differential expression comparison made. Ten DE plasma miRNAs were identified as homologous between mice and human CCM1 and CCM3 genotypes. miR-375-5p was upregulated in CCM1 patients and Ccm1 mice as well as in CCM3 patients and Ccm3 mice compared to healthy controls (HC) and wild type (WT) mice. miR-93-5p was downregulated in CCM3 patients and Ccm3 mice compared to HC and WT mice. It was also downregulated in CCM1 patients and Ccm1 mice compared to CCM3 patients and Ccm3 mice. miR-20b-5p was upregulated in CCM1 patients and Ccm1 mice compared to HC and WT mice. miR-370-3p, miR-487b-3p, and miR-369-3p were upregulated in CCM3 patients and Ccm3 mice compared to HC and WT mice. miR-410-3p and miR-323-3p were upregulated in CCM1 patients and Ccm1 mice compared to CCM3 patients and Ccm3 mice. miR-128-3p and miR-9-5p were upregulated in Ccm1 compared to Ccm3 in mice but downregulated in CCM1 compared to CCM3 in patients.

FIG. 3. Homologous Differentially Expressed (DE) Plasma miRNAs of CCM1 and CCM3 Target Genes Associated with CCM-Enriched KEGG Pathways. Seven of the ten homologous DE plasma miRNAs identified between mice and humans target DE genes [|FC|>1.5; p<0.05, FDR corrected] within the previously published transcriptomes of (1) laser micro-dissected NVUs of human surgically resected CCM lesions, and/or (2) in vitro Ccm1^{-/-} mouse BMECs, (2) in vitro Ccm3/mouse BMECs, as well as within (3) laser micro-dissected lesional NVUs of Ccm3^{+/-} mice. The color-coded enriched KEGG pathways [p<0.05, FDR corrected; Bayes factor≥3] presented were limited to (1) those related to CCM processes identified through a comprehensive literature search and (2) linked to at least two homologous DE miRNAs. These biological processes include: (1) Neuron, Glia, Pericyte Function, (2) Permeability/Adhesion, (3) Apoptosis and Oxidative Stress, (4) Inflammation/Immune Response, (5) Vascular Processes, (6) Cellular Proliferation. Additionally, only DE genes linked to these KEGG pathways are shown. Homologous DE miRNAs are shown with a gradual increase in size for an increasing number of connections to DE genes.

FIG. 4a-f. Relative Quantification of Differentially Expressed (DE) miRNAs through RT-qPCR. Validation of differential expression was performed on a select panel of DE miRNAs in an independent cohort of familial-CCM patients along with healthy non-CCM patients using RT-qPCR. Relative expression showed that plasma levels of these miRNAs followed the same directionality of dysregulation seen in the miRNome sequencing results. (a) miR-20b-5p levels were higher in non-CCM subjects and CCM3 patients compared to CCM1 patients, (b) miR-93-5p levels were higher in CCM1 and CCM3 patients compared to non-CCM subjects, (c) miR-9-5p levels were higher in non-CCM subjects and CCM1 patients compared to CCM3 patients, (d) miR-375-3p levels were higher in CCM1 and CCM3 patients compared to non-CCM subjects, (e) miR-128-3p showed no significant differences between groups, (f) let-7e-5p levels were higher in CCM1 and CCM3 patients compared to non-CCM subjects. Statistical analyses were performed using a Mann-Whitney test. * p<0.05; ** p<0.01; NS=not significant

FIG. 5a-d. Integrated Plasma miRNA and Protein Biomarker and Diagnosis of Familial CCM. Two machine learning approaches were applied to integrate plasma miRNAs and proteins into a diagnostic biomarker for CCM and identify the best model with the lowest Akaike information criterion. (a) Using a 10-fold cross validation method, the best weighted combination of molecules to diagnose familial CCM included plasma relative quantification values of let-7e-5p, miR-93-5p, miR-20b-5p, miR-128-3p, and plasma concentrations of interleukin-10 (IL-10) and thrombospondin-2 (Tsp-2). (b) Receiver operating characteristic (ROC) analysis for this model yielded a 97.5% area under the curve (AUC), with a sensitivity of 94.7% and specificity of 100%. The red curve represents the average ROC of the cross validation, while the dash-dotted lines represent the unique ROC curves generated for each of the 10 iterations within the 10-fold cross validation. (c) Using a leave-one-out cross validation method, the best weighted combination of molecules to diagnose familial CCM included plasma relative quantification values of miR-93-5p, miR-9-5p, and plasma concentrations of interleukin-2 (IL-2), tumor necrosis factor receptor 1 (TNFRI), and Tsp-2. (d) ROC analysis for this model yielded a 97.4% AUC, with a sensitivity of 94.7% and a specificity of 100%. [X]ᵣ denotes the relative quantification value for each miRNA. Statistical analyses to compare the canonical values were implemented using an unpaired two samples Student's t-test, assessed with pooled standard deviation, or Mann-Whitney test according to the equality of the variance. *** p<0.001

FIG. 6. Methodological Overview for Integrated Plasma miRNA and Protein Biomarker Development. A feature selection approach using machine learning cross validations was performed on miRNAs and proteins to select the best independent molecules. The feature selected miRNAs were used to develop the best miRNA biomarker across 37 patients, and similarly the feature selected proteins were used to develop the best protein biomarker across 74 patients. A cohort of 25 patients with both miRNAs and proteins was used to develop the best combined biomarker to diagnose familial-CCM patients. Feature selected miRNAs or proteins were considered in this combined biomarker. Finally, the best miRNA biomarker, and the best protein biomarker were tested on the same 25 patient cohort to compare performance across all three models.

FIG. 7a-f. Absolute Quantification of Differentially Expressed (DE) miRNAs through RT-qPCR. Validation of differential expression was performed on a select panel of DE miRNAs in an independent cohort of 12 CCM3, 12 CCM1, and 13 healthy non-CCM patients using RT-qPCR. (a) miR-20b-5p levels were higher in CCM1 patients compared to CCM3 patients and non-CCM subjects, (b) miR-93-5p showed no significant differences between groups, (c) miR-9-5p levels were lower in CCM3 patients compared to CCM1 patients, (d) miR-375-3p levels were higher in CCM1 and CCM3 patients compared to non-CCM subjects, (e) miR-128-3p showed no significant differences between groups, (f) let-7e-5p levels were higher in CCM1 patients compared to non-CCM subjects.* p<0.05; NS=not significant

FIG. 8a-d. Plasma miRNA Biomarker and Diagnosis of Familial CCM. (a and b) Both using a 10-fold or leave-one-out cross validation method yielded the same best weighted combination of miRNAs to diagnose familial CCM, which included relative quantification values of let-7e-5p and miR-93-5p. (c and d) Receiver operating characteristic analysis for this model yielded a 75.4% area under the curve (AUC), with a sensitivity of 84% and a specificity of 67%.

FIG. 9a-d. Plasma Protein Biomarker and Diagnosis of Familial CCM. (a) Using a 10-fold cross validation method, the best weighted combination of proteins to diagnose familial CCM included concentrations of interleukin-10 (IL-10), VEGF, and thrombospondin-2 (Tsp-2). (b) Receiver operating characteristic (ROC) analysis for this model yielded a 77.2% area under the curve (AUC), with a sensitivity of 79% and a specificity of 67%. (c) Using a leave-one-out cross validation method, the best weighted combination of proteins to diagnose familial CCM included concentrations of IL-2, TNFRI, and Tsp-2 (d) ROC analysis for this model yielded a 96.5% AUC, with a sensitivity of 89.5% and a specificity of 100%. p<0.001

FIG. 10a-f. Integrated Plasma miRNA and Protein Biomarkers Selected Through 10-fold Cross Validation and Diagnosis of CCM Genotypes. (a) Using a 10-fold cross validation method, the best weighted combination of miRNAs and proteins to diagnose CCM1 patients from non-CCM subjects included concentrations of let-7e-5p, miR-93-5p, and thrombomodulin (TM). (b) Receiver operating characteristic (ROC) analysis for this model yielded an 89.6% area under the curve (AUC), with a sensitivity of 75% and a specificity of 67%. (c) Using a 10-fold cross validation method, the best weighted combination of miRNAs and proteins to diagnose CCM3 patients from non-CCM subjects included concentrations of miR-93-5p, miR-128-3p, miR-375-3p, CCL2, TNFRI, and thrombospondin-2 (Tsp-2). (d) ROC analysis for this model yielded a 100% AUC, with a sensitivity of 91% and a specificity of 83%. (e) Using a 10-fold cross validation method, the best weighted combination of miRNAs and proteins to diagnose CCM1 from CCM3 patients included concentrations of let-7e-5p, miR-9-5p, and IL-6. (f) ROC analysis for this model yielded a 90.9% AUC, with a sensitivity of 75% and a specificity of 82%. [X]ᵣ denotes the relative quantification value for each miRNA. *p<0.05; ** p<0.01; *** p<0.001.

FIG. 11a-f. Integrated Plasma miRNA and Protein Biomarkers Selected Through Leave-One-Out Cross Validation and Diagnosis of CCM Genotypes. (a) Using a leave-one-out cross validation method, the best weighted combination of miRNAs and proteins to diagnose CCM1 patients from non-CCM subjects included concentrations of miR-128-3p, and IL-6. (b) Receiver operating characteristic (ROC) analysis for this model yielded an 87.5% area under the curve (AUC), with a sensitivity of 100% and a specificity of 75%. (c) Using a leave-one-out cross validation method, the best weighted combination of miRNAs and proteins to diagnose CCM3 patients from non-CCM subjects included concentrations of miR-20b-5p and TNFRI. (d) ROC analysis for this model yielded a 90.9% AUC, with a sensitivity of 83% and a specificity of 91%. (e) Using a leave-one-out cross validation method, the best weighted combination of proteins to diagnose CCM1 from CCM3 patients included concentrations of C-reactive protein (CRP), IL-6, and thrombospondin-2 (Tsp-2). (f) ROC analysis for this model yielded a 90.9% AUC, with a sensitivity of 100% and a specificity of 82%. [X]ᵣ denotes the relative quantification value for each miRNA. * p<0.05; ** p<0.01; *** p<0.001.

### DETAILED DESCRIPTION OF THE INVENTION

The inventors identified and validated several homologous DE plasma miRNAs with mechanistic links to CCM disease in preclinical murine models and CCM patients. Additionally, they show that the integration of readily measurable plasma proteins and miRNAs into a combined biomarker can diagnose familial-CCM disease with 100% accuracy. To the knowledge of the inventors, such a translational approach to biomarker discovery in mouse models and humans, transcriptomic links, validation in an independent human cohort, and the combination of miRNA and protein levels to optimize biomarker performance have not been previously described in any other disease.

This study is the first to show a near perfect diagnostic precision with the weighted combination of plasma miRNAs and proteins in a Mendelian disease, based on ML and Bayesian approaches. Two cross-validation methods confirmed that integrating plasma miRNAs and proteins improves the diagnostic association with familial-CCM compared to each circulating compound (e.g., plasma miRNA and protein) alone. This result acts as an important proof of concept for integration of several types of molecules in plasma biomarker development (12). A homologous biomarker in mice and humans, which can differentiate familial-CCM with germline loss of function mutations can be immediately applied in preclinical studies and early trials, monitoring effectiveness of gene restoration therapeutics, which are currently of great interest. As patients undergo gene therapy, one could monitor their progress and the duration of therapeutic effect as biomarker canonical values shift in comparison to a healthy non-CCM profile. This approach to combined biomarker development is readily applicable to other clinical contexts of use, including the prediction of new lesion formation.

In summary, the inventors have shown for the first time in any disease, that homologous DE plasma miRNAs can reflect the germline loss of function mutation in preclinical murine models and patients. They have also shown that plasma miRNAs can distinguish cases who would develop new lesions and can be assayed in peripheral blood. The inventors describe a novel approach which can be developed for translatable circulating biomarkers in specific clinical contexts of use in CCM and other Mendelian diseases.

II. ROC analysis

In statistics, a receiver operating characteristic (ROC), or ROC curve, is a graphical plot that illustrates the performance of a binary classifier system as its discrimination threshold is varied. ROC analysis may be applied to determine a cut-off value or threshold setting of biomarker expression, such as the canonical value described herein. For example, patients with biological samples determined to have biomarker expression value above a certain cut-off threshold but below a higher cut-off threshold may be determined to have endometriosis. Patients with biological samples determined to have a biomarker expression level that surpasses the cut-off threshold may be determined to have a disease or condition such as multiple sclerosis. The curve is created by plotting the true positive rate against the false positive rate at various threshold settings. (The true-positive rate is also known as sensitivity in biomedical informatics, or recall in machine learning. The false-positive rate is also known as the fall-out and can be calculated as 1-specificity). The ROC curve is thus the sensitivity as a function of fall-out. In general, if the probability distributions for both detection and false alarm are known, the ROC curve can be generated by plotting the cumulative distribution function (area under the probability distribution from—infinity to + infinity) of the detection probability in the y-axis versus the cumulative distribution function of the false-alarm probability in x-axis.

ROC analysis provides tools to select possibly optimal models and to discard suboptimal ones independently from (and prior to specifying) the cost context or the class distribution. ROC analysis is related in a direct and natural way to cost/benefit analysis of diagnostic decision making.

The ROC curve was first developed by electrical engineers and radar engineers during World War II for detecting enemy objects in battlefields and was soon introduced to psychology to account for perceptual detection of stimuli. ROC analysis since then has been used in medicine, radiology, biometrics, and other areas for many decades and is increasingly used in machine learning and data mining research.

The ROC is also known as a relative operating characteristic curve, because it is a comparison of two operating characteristics (TPR and FPR) as the criterion changes. ROC analysis curves are known in the art and described in Metz CE (1978) Basic principles of ROC analysis. Seminars in Nuclear Medicine 8:283-298; Youden W J (1950) An index for rating diagnostic tests. Cancer 3:32-35; Zweig MH, Campbell G (1993) Receiver-operating characteristic (ROC) plots: a fundamental evaluation tool in clinical medicine. Clinical Chemistry 39:561-577; and Greiner M, Pfeiffer D, Smith RD (2000) Principles and practical application of the receiver-operating characteristic analysis for diagnostic tests. Preventive Veterinary Medicine 45:23-41, which are herein incorporated by reference in their entirety. A ROC analysis may be used to create cut-off values for prognosis and/or diagnosis purposes.

### III. Gene and RNA Expression Levels

Methods disclosed herein include measuring expression of genes and/or RNAs (RNAs) such as messenger RNAs (mRNAs), micro RNAs (miRNAs) and noncoding RNAs (ncRNAs). Measurement of expression can be done by a number of processes known in the art. The process of measuring expression may begin by extracting RNA from a metastasis tissue sample. Extracted mRNA and/or ncRNA can be detected by hybridization (for example by means of Northern blot analysis or DNA or RNA arrays (microarrays) after converting RNA into labeled cDNA) and/or amplification by means of a enzymatic chain reaction. Quantitative or semi-quantitative enzymatic amplification methods such as polymerase chain reaction (PCR) or quantitative real-time RT-PCR or semi-quantitative RT-PCR techniques can be used. Suitable primers for amplification methods encompassed herein can be readily designed by a person skilled in the art. Other amplification methods include ligase chain reaction (LCR), transcription-mediated amplification (TMA), strand displacement amplification (SDA), isothermal amplification of nucleic acids, and nucleic acid sequence based amplification (NASBA). Expression levels of mRNAs and/or ncRNAs may also be measured by RNA sequencing methods known in the art. RNA sequencing methods may include mRNA-seq, total RNA-seq, targeted RNA-seq, small RNA-seq, single-cell RNA-seq, ultra-low-input RNA-seq, RNA exome capture sequencing, and ribosome profiling. Sequencing data may be processed an aligned using methods known in the art.

To normalize the expression values of one gene among different samples, comparing the mRNA and/or ncRNA level of interest in the samples from the subject object of study with a control RNA level is possible. As it is used herein, a “control RNA” is an RNA of a gene for which the expression level does not differ among different metastatic subtypes, for example a gene that is constitutively expressed in all types of cells. A control RNA is preferably an mRNA derived from a housekeeping gene encoding a protein that is constitutively expressed and carrying out essential cell functions. A known amount of a control RNA may be added to the sample(s) and the value measured for the level of the RNA of interest may be normalized to the value measured for the known amount of the control RNA. Normalization for some methods, such as for sequencing, may comprise calculating the reads per kilobase of transcript per million mapped reads (RPKM) for a gene of interest, or may comprise calculating the fragments per kilobase of transcript per million mapped reads (FPKM) for a gene of interest. Normalization methods may comprise calculating the log 2-transformed count per million (log-CPM). It can be appreciated to one skilled in the art that any method of normalization that accurately calculates the expression value of an RNA for comparison between samples may be used.

Methods disclosed herein may include comparing a measured expression level to a reference expression level. The term “reference expression level” refers to a value used as a reference for the values/data obtained from samples obtained from patients. The reference level can be an absolute value, a relative value, a value which has an upper and/or lower limit, a series of values, an average value, a median, a mean value, or a value expressed by reference to a control or reference value. A reference level can be based on the value obtained from an individual sample, such as, for example, a value obtained from a sample from the subject object of study but obtained at a previous point in time. The reference level can be based on a high number of samples, such as the levels obtained in a cohort of subjects having a particular characteristic. The reference level may be defined as the mean level of the patients in the cohort. For example, the reference expression level for a gene or RNA can be based on the mean expression level of the gene or RNA obtained from a number of patients who have SNF2 metastases. A reference level can be based on the expression levels of the markers to be compared obtained from samples from subjects who do not have a disease state or a particular phenotype. The person skilled in the art will see that the particular reference expression level can vary depending on the specific method to be performed.

The methods may include determining that a measured expression level is higher than, lower than, increased relative to, decreased relative to, equal to, or within a predetermined amount of a reference expression level. A higher, lower, increased, or decreased expression level may be at least 1, 1.5, 2, 2.5, 3, 3.5, 4, 4.5, 5, 5.5, 6, 7, 8, 9, 10, 12, 14, 16, 18, 20, 50, 100, 150, 200, 250, 500, or 1000 fold (or any derivable range therein) or at least 10, 20, 30, 40, 50, 60, 70, 80, 90, 100, 200, 300, 400, 500, 600, 700, 800, or 900% different than the reference level, or any derivable range therein. These values may represent a predetermined threshold level, and may include determining that the measured expression level is higher by a predetermined amount or lower by a predetermined amount than a reference level. A level of expression may be qualified as “low” or “high,” which indicates the patient expresses a certain gene or RNA at a level relative to a reference level or a level with a range of reference levels that are determined from multiple samples meeting particular criteria. The level or range of levels in multiple control samples is an example of this. A certain level or a predetermined threshold value may be at, below, or above 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percentile, or any range derivable therein. Moreover, a threshold level may be derived from a cohort of individuals meeting a particular criteria. The number in the cohort may be, be at least, or be at most 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 441, 450, 460, 470, 480, 490, 500, 510, 520, 530, 540, 550, 560, 570, 580, 590, 600, 700, 800, 900, 1000, 1100, 1200, 1300, 1400, 1500, 1600, 1700, 1800, 1900, 2000 or more (or any range derivable therein). A measured expression level can be considered equal to a reference expression level if it is within a certain amount of the reference expression level, and such amount may be an amount that is predetermined. This can be the case, for example, when a classifier is used to identify the molecular subtype of a metastasis. The predetermined amount may be within 0.1, 0.2, 0.3, 0.4, 0.5, 0.6, 0.7, 0.8, 0.9, 1.0, 2, 3, 4, 5, 6, 7, 8, 9, 10, 11, 12, 13, 14, 15, 16, 17, 18, 19, 20, 21, 22, 23, 24, 25, 30, 35, 40, 45, or 50% of the reference level, or any range derivable therein.

For any comparison of gene and/or RNA expression levels to a mean expression levels or a reference expression levels, the comparison is to be made on a gene-by-gene and RNA-by-RNA basis. For example, if the expression levels of gene A, gene B, and miRNA X in a patient's metastasis are measured, a comparison to mean expression levels in metastases of a cohort of patients would involve: comparing the expression level of gene A in the patient's metastasis with the mean expression level of gene A in metastases of the cohort of patients, comparing the expression level of gene B in the patient's metastasis with the mean expression level of gene B in metastases of the cohort of patients, and comparing the expression level of RNA X in the patient's metastasis with the mean expression level of RNA X in metastases of the cohort of patients. Comparisons that involve determining whether the expression level measured in a patient's metastasis is within a predetermined amount of a mean expression level or reference expression level are similarly done on a gene-by-gene and ncRNA-by-ncRNA basis, as applicable.

### IV. Nucleic Acid Modifications

The oligonucleotides of the disclosure, such as the miRNA modulators and other nucleic acids described herein may have modifications that increase the stability of the nucleic acid. The modulator may be an oligonucleotide analog. The term “oligonucleotide analog” refers to compounds which function like oligonucleotides but which have non-naturally occurring portions. Oligonucleotide analogs can have altered sugar moieties, altered base moieties or altered inter-sugar linkages. The term “oligomers” is intended to encompass oligonucleotides, oligonucleotide analogs or oligonucleosides. Thus, in speaking of “oligomers” reference is made to a series of nucleosides or nucleoside analogs that are joined via either natural phosphodiester bonds or other linkages, including the four atom linkers. Although the linkage generally is from the 3′ carbon of one nucleoside to the 5′ carbon of a second nucleoside, the term “oligomer” can also include other linkages such as 2′-5′ linkages.

Oligonucleotide analogs also can include other modifications, particularly modifications that increase nuclease resistance, improve binding affinity, and/or improve binding specificity. For example, when the sugar portion of a nucleoside or nucleotide is replaced by a carbocyclic moiety, it is no longer a sugar. Moreover, when other substitutions, such a substitution for the inter-sugar phosphodiester linkage are made, the resulting material is no longer a true nucleic acid species. All such compounds are considered to be analogs. Throughout this specification, reference to the sugar portion of a nucleic acid species shall be understood to refer to either a true sugar or to a species taking the structural place of the sugar of wild type nucleic acids. Moreover, reference to inter-sugar linkages shall be taken to include moieties serving to join the sugar or sugar analog portions in the fashion of wild type nucleic acids.

The present disclosure concerns modified oligonucleotides, i.e., oligonucleotide analogs or oligonucleosides, and methods for effecting the modifications. These modified oligonucleotides and oligonucleotide analogs may exhibit increased chemical and/or enzymatic stability relative to their naturally occurring counterparts. Extracellular and intracellular nucleases generally do not recognize and therefore do not bind to the backbone-modified compounds. When present as the protonated acid form, the lack of a negatively charged backbone may facilitate cellular penetration.

The modified internucleoside linkages are intended to replace naturally-occurring phosphodiester-5′-methylene linkages with four atom linking groups to confer nuclease resistance and enhanced cellular uptake to the resulting compound. Preferred linkages have structure CH2—RA—NR1 CH2, CH2—NR1—RA—CH2, RA—NR1—CH2—CH2, CH2—CH2—NR1—RA, CH2—CH2—RA—NR1, or NR1—RA—CH2—CH2 where RA is O or NR2.

Modifications may be achieved using solid supports which may be manually manipulated or used in conjunction with a DNA synthesizer using methodology commonly known to those skilled in DNA synthesizer art. Generally, the procedure involves functionalizing the sugar moieties of two nucleosides which will be adjacent to one another in the selected sequence. In a 5′ to 3′ sense, an “upstream” synthon is modified at its terminal 3′ site, while a “downstream” synthon is modified at its terminal 5′ site.

Oligonucleosides linked by hydrazines, hydroxylarnines, and other linking groups can be protected by a dimethoxytrityl group at the 5′-hydroxyl and activated for coupling at the 3′-hydroxyl with cyanoethyldiisopropyl-phosphite moieties. These compounds can be inserted into any desired sequence by standard, solid phase, automated DNA synthesis techniques. One of the most popular processes is the phosphoramidite technique. Oligonucleotides containing a uniform backbone linkage can be synthesized by use of CPG-solid support and standard nucleic acid synthesizing machines such as Applied Biosystems Inc. 380B and 394 and Milligen/Biosearch 7500 and 8800s. The initial nucleotide (number 1 at the 3′-terminus) is attached to a solid support such as controlled pore glass. In sequence specific order, each new nucleotide is attached either by manual manipulation or by the automated synthesizer system.

Free amino groups can be alkylated with, for example, acetone and sodium cyanoboro hydride in acetic acid. The alkylation step can be used to introduce other, useful, functional molecules on the macromolecule. Such useful functional molecules include but are not limited to reporter molecules, RNA cleaving groups, groups for improving the pharmacokinetic properties of an oligonucleotide, and groups for improving the pharmacodynamic properties of an oligonucleotide. Such molecules can be attached to or conjugated to the macromolecule via attachment to the nitrogen atom in the backbone linkage. Alternatively, such molecules can be attached to pendent groups extending from a hydroxyl group of the sugar moiety of one or more of the nucleotides. Examples of such other useful functional groups are provided by WO1993007883, which is herein incorporated by reference, and in other of the above-referenced patent applications.

Solid supports may include any of those known in the art for polynucleotide synthesis, including controlled pore glass (CPG), oxalyl controlled pore glass [53], TentaGel Support—an aminopolyethyleneglycol derivatized support or Poros-a copolymer of polystyrene/divinylbenzene. Attachment and cleavage of nucleotides and oligonucleotides can be effected via standard procedures [55]. As used herein, the term solid support further includes any linkers (e.g., long chain alkyl amines and succinyl residues) used to bind a growing oligonucleoside to a stationary phase such as CPG.

### A. Locked Nucleotides

The nucleic acid of the disclosure, such as the biomarker modulator may comprise a locked nucleic acid. A locked nucleic acid (LNA or Ln), also referred to as inaccessible RNA, is a modified RNA nucleotide. The ribose moiety of an LNA nucleotide is modified with an extra bridge connecting the 2′ oxygen and 4′ carbon. The bridge “locks” the ribose in the 3′-endo (North) conformation, which is often found in the A-form duplexes. LNA nucleotides can be mixed with DNA or RNA residues in the oligonucleotide whenever desired and hybridize with DNA or RNA according to Watson-Crick base-pairing rules. Such oligomers are synthesized chemically and are commercially available. The locked ribose conformation enhances base stacking and backbone pre-organization. This significantly increases the hybridization properties (melting temperature) of oligonucleotides.

### B. Ethylene Bridged Nucleotides

The nucleic acid of the disclosure, such as the biomarker modulator may comprise one or more ethylene bridged nucleotides. Ethylene-bridged nucleic acids (ENA or En) are modified nucleotides with a 2′-0, 4′C ethylene linkage. Like locked nucleotides, these nucleotides also restrict the sugar puckering to the N-conformation of RNA.

### C. Peptide Nucleic Acids

The nucleic acid of the disclosure, such as the biomarker modulator may comprise one or more peptide nucleic acids. Peptide nucleic acids (PNA or Pn) mimic the behavior of DNA and binds complementary nucleic acid strands. The term, “peptide,” when used herein may also refer to a peptide nucleic acid. PNA is an artificially synthesized polymer similar to DNA or RNA. DNA and RNA have a deoxyribose and ribose sugar backbone, respectively, whereas PNA's backbone is composed of repeating N-(2-aminoethyl)-glycine units linked by peptide bonds. The various purine and pyrimidine bases are linked to the backbone by a methylene bridge (—CH2—) and a carbonyl group (—(C═O)—). PNAs are depicted like peptides, with the N-terminus at the first (left) position and the C-terminus at the last (right) position.

Since the backbone of PNAs contains no charged phosphate groups, the binding between PNA/DNA strands is stronger than between DNA/DNA strands due to the lack of electrostatic repulsion. PNAs are not easily recognized by either nucleases or proteases, making them resistant to degradation by enzymes. PNAs are also stable over a wide pH range. The PNAs described herein may have improved cytosolic delivery over other PNAs.

D. 5′ (E)-vinyl-phosphonate (VP) modification

The nucleic acid of the disclosure, such as the biomarker modulator may comprise one or more 5′ (E)-vinyl-phosphonate (VP) modifications. 5′-Vinyl-phosphonate modifications (metabolically stable phosphate mimics) have been reported to enhance the metabolic stability and the potency of oligonucleotides.

### E. Morpholinos

The nucleic acid of the disclosure, such as the biomarker modulator may comprise a morpholino. Morpholinos are synthetic molecules that are the product of a redesign of natural nucleic acid structure. Usually 25 bases in length, they bind to complementary sequences of RNA or single-stranded DNA by standard nucleic acid base-pairing. In terms of structure, the difference between Morpholinos and DNA is that, while Morpholinos have standard nucleic acid bases, those bases are bound to methylenemorpholine rings linked through phosphorodiamidate groups instead of phosphates. The figure compares the structures of the two strands depicted there, one of RNA and the other of a Morpholino. Replacement of anionic phosphates with the uncharged phosphorodiamidate groups eliminates ionization in the usual physiological pH range, so Morpholinos in organisms or cells are uncharged molecules. The entire backbone of a Morpholino is made from these modified subunits.

### V. Delivery Vehicles

The current disclosure contemplates several delivery systems compatible with nucleic acids that provide for roughly uniform distribution and have controllable rates of release. A variety of different media are described below that are useful in creating nucleic acid delivery systems. It is not intended that any one medium or carrier is limiting to the present invention. Note that any medium or carrier may be combined with another medium or carrier; for example, a polymer microparticle carrier attached to a compound may be combined with a gel medium.

Carriers or mediums contemplated by this disclosure comprise a material selected from the group comprising gelatin, collagen, cellulose esters, dextran sulfate, pentosan polysulfate, chitin, saccharides, albumin, fibrin sealants, synthetic polyvinyl pyrrolidone, polyethylene oxide, polypropylene oxide, block polymers of polyethylene oxide and polypropylene oxide, polyethylene glycol, acrylates, acrylamides, methacrylates including, but not limited to, 2-hydroxyethyl methacrylate, poly(ortho esters), cyanoacrylates, gelatin-resorcinol-aldehyde type bioadhesives, polyacrylic acid and copolymers and block copolymers thereof.

### A. Microparticles

Also described is a delivery system comprising a microparticle. Preferably, microparticles comprise liposomes, nanoparticles, microspheres, nanospheres, microcapsules, and nanocapsules. Preferably, some microparticles contemplated by the present invention comprise poly(lactide-co-glycolide), aliphatic polyesters including, but not limited to, poly-glycolic acid and poly-lactic acid, hyaluronic acid, modified polysaccharides, chitosan, cellulose, dextran, polyurethanes, polyacrylic acids, pseudo-poly(amino acids), polyhydroxybutyrate-related copolymers, polyanhydrides, polymethylmethacrylate, poly(ethylene oxide), lecithin and phospholipids.

### B. Liposomes

Liposomes capable of attaching and releasing nucleic acids conjugates, polypeptides, and fusion proteins may be used in the methods of the disclosure. Liposomes are microscopic spherical lipid bilayers surrounding an aqueous core that are made from amphiphilic molecules such as phospholipids. For example, a liposome may trap a nucleic acid between the hydrophobic tails of the phospholipid micelle. Water soluble agents can be entrapped in the core and lipid-soluble agents can be dissolved in the shell-like bilayer. Liposomes have a special characteristic in that they enable water soluble and water insoluble chemicals to be used together in a medium without the use of surfactants or other emulsifiers. Liposomes can form spontaneously by forcefully mixing phospholipids in aqueous media. Water soluble compounds are dissolved in an aqueous solution capable of hydrating phospholipids. Upon formation of the liposomes, therefore, these compounds are trapped within the aqueous liposomal center. The liposome wall, being a phospholipid membrane, holds fat soluble materials such as oils. Liposomes provide controlled release of incorporated compounds. In addition, liposomes can be coated with water soluble polymers, such as polyethylene glycol to increase the pharmacokinetic half-life. Also contemplated is an ultra high-shear technology to refine liposome production, resulting in stable, unilamellar (single layer) liposomes having specifically designed structural characteristics. These unique properties of liposomes allow the simultaneous storage of normally immiscible compounds and the capability of their controlled release.

The disclosure contemplates cationic and anionic liposomes, as well as liposomes having neutral lipids. Preferably, cationic liposomes comprise negatively-charged materials by mixing the materials and fatty acid liposomal components and allowing them to charge-associate. Clearly, the choice of a cationic or anionic liposome depends upon the desired pH of the final liposome mixture. Examples of cationic liposomes include lipofectin, lipofectamine, and lipofectace.

Also contemplated is a delivery system comprising liposomes that provides controlled release of at least one molecule described herein. Preferably, liposomes that are capable of controlled release: i) are biodegradable and non-toxic; ii) carry both water and oil soluble compounds; iii) solubilize recalcitrant compounds; iv) prevent compound oxidation; v) promote protein stabilization; vi) control hydration; vii) control compound release by variations in bilayer composition such as, but not limited to, fatty acid chain length, fatty acid lipid composition, relative amounts of saturated and unsaturated fatty acids, and physical configuration; viii) have solvent dependency; iv) have pH-dependency and v) have temperature dependency.

The compositions of liposomes are broadly categorized into two classifications. Conventional liposomes are generally mixtures of stabilized natural lecithin (PC) that may comprise synthetic identical-chain phospholipids that may or may not contain glycolipids. Special liposomes may comprise: i) bipolar fatty acids; ii) the ability to attach antibodies for tissue-targeted therapies; iii) coated with materials such as, but not limited to lipoprotein and carbohydrate; iv) multiple encapsulation and v) emulsion compatibility.

Liposomes may be easily made in the laboratory by methods such as, but not limited to, sonication and vibration. Alternatively, compound-delivery liposomes are commercially available. For example, Collaborative Laboratories, Inc. are known to manufacture custom designed liposomes for specific delivery requirements.

### C. Microspheres, Microparticles and Microcapsules

Microspheres and microcapsules are useful due to their ability to maintain a generally uniform distribution, provide stable controlled compound release and are economical to produce and dispense. Preferably, an associated delivery gel or the compound-impregnated gel is clear or, alternatively, said gel is colored for easy visualization by medical personnel.

Microspheres are obtainable commercially (Prolease™, Alkerme's: Cambridge, Mass.). For example, a freeze dried medium comprising at least one therapeutic agent is homogenized in a suitable solvent and sprayed to manufacture microspheres in the range of 20 to 90 μm Techniques are then followed that maintain sustained release integrity during phases of purification, encapsulation and storage. Scott et al., Improving Protein Therapeutics With Sustained Release Formulations, Nature Biotechnology, Volume 16:153-157 (1998). Modification of the microsphere composition by the use of biodegradable polymers can provide an ability to control the rate of nucleic acid release. Miller et al., Degradation Rates of Oral Resorbable Implants {Polylactates and Polyglycolates: Rate Modification and Changes in PLA/PGA Copolymer Ratios, J. Biomed. Mater. Res., Vol. 11:711-719 (1977).

Alternatively, a sustained or controlled release microsphere preparation is prepared using an in-water drying method, where an organic solvent solution of a biodegradable polymer metal salt is first prepared. Subsequently, a dissolved or dispersed medium of a nucleic acid is added to the biodegradable polymer metal salt solution. The weight ratio of a nucleic acid to the biodegradable polymer metal salt may for example be about 1:100000 to about 1:1, preferably about 1:20000 to about 1:500 and more preferably about 1:10000 to about 1:500. Next, the organic solvent solution containing the biodegradable polymer metal salt and nucleic acid is poured into an aqueous phase to prepare an oil/water emulsion. The solvent in the oil phase is then evaporated off to provide microspheres. Finally, these microspheres are then recovered, washed and lyophilized. Thereafter, the microspheres may be heated under reduced pressure to remove the residual water and organic solvent.

Other methods useful in producing microspheres that are compatible with a biodegradable polymer metal salt and nucleic acid mixture are: i) phase separation during a gradual addition of a coacervating agent; ii) an in-water drying method or phase separation method, where an antiflocculant is added to prevent particle agglomeration and iii) by a spray-drying method.

The present invention contemplates a medium comprising a microsphere or microcapsule capable of delivering a controlled release of a nucleic acid for a duration of approximately between 1 day and 6 months. The microsphere or microparticle may be colored to allow the medical practitioner the ability to see the medium clearly as it is dispensed. The microsphere or microcapsule may be clear. The microsphere or microparticle may be impregnated with a radio-opaque fluoroscopic dye.

Controlled release microcapsules may be produced by using known encapsulation techniques such as centrifugal extrusion, pan coating and air suspension. Such microspheres and/or microcapsules can be engineered to achieve desired release rates. For example, Oliosphere™ (Macromed) is a controlled release microsphere system. These particular microsphere's are available in uniform sizes ranging between 5-500 μm and composed of biocompatible and biodegradable polymers. Specific polymer compositions of a microsphere can control the nucleic acid release rate such that custom-designed microspheres are possible, including effective management of the burst effect. ProMaxx™ (Epic Therapeutics, Inc.) is a protein-matrix delivery system. The system is aqueous in nature and is adaptable to standard pharmaceutical delivery models. In particular, ProMaxx™ are bioerodible protein microspheres that deliver both small and macromolecular drugs, and may be customized regarding both microsphere size and desired release characteristics.

A microsphere or microparticle may comprise a pH sensitive encapsulation material that is stable at a pH less than the pH of the internal mesentery. The typical range in the internal mesentery is pH 7.6 to pH 7.2. Consequently, the microcapsules should be maintained at a pH of less than 7. However, if pH variability is expected, the pH sensitive material can be selected based on the different pH criteria needed for the dissolution of the microcapsules. The encapsulated nucleic acid, therefore, will be selected for the pH environment in which dissolution is desired and stored in a pH preselected to maintain stability. Examples of pH sensitive material useful as encapsulants are Eudragit™ L-100 or S-100 (Rohm GMBH), hydroxypropyl methylcellulose phthalate, hydroxypropyl methylcellulose acetate succinate, polyvinyl acetate phthalate, cellulose acetate phthalate, and cellulose acetate trimellitate. Lipids may comprise the inner coating of the microcapsules. In these compositions, these lipids may be, but are not limited to, partial esters of fatty acids and hexitiol anhydrides, and edible fats such as triglycerides. Lew C. W., Controlled-Release pH Sensitive Capsule And Adhesive System And Method. U.S. Pat. No. 5,364,634 (herein incorporated by reference).

The present invention contemplates a microparticle comprising a gelatin, or other polymeric cation having a similar charge density to gelatin (i.e., poly-L-lysine) and is used as a complex to form a primary microparticle. A primary microparticle is produced as a mixture of the following composition: i) Gelatin (60 bloom, type A from porcine skin), ii) chondroitin 4-sulfate (0.005%-0.1%), iii) glutaraldehyde (25%, grade 1), and iv) 1-ethyl-3-(3-dimethylaminopropyl)-carbodiimide hydrochloride (EDC hydrochloride), and ultra-pure sucrose (Sigma Chemical Co., St. Louis, Mo.). The source of gelatin is not thought to be critical; it can be from bovine, porcine, human, or other animal source. Typically, the polymeric cation is between 19,000-30,000 daltons. Chondroitin sulfate is then added to the complex with sodium sulfate, or ethanol as a coacervation agent.

Following the formation of a microparticle, a nucleic acid is directly bound to the surface of the microparticle or is indirectly attached using a “bridge” or “spacer”. The amino groups of the gelatin lysine groups are easily derivatized to provide sites for direct coupling of a compound. Alternatively, spacers (i.e., linking molecules and derivatizing moieties on targeting ligands) such as avidin-biotin are also useful to indirectly couple targeting ligands to the microparticles. Stability of the microparticle is controlled by the amount of glutaraldehyde-spacer crosslinking induced by the EDC hydrochloride. A controlled release medium is also empirically determined by the final density of glutaraldehyde-spacer crosslinks.

The present invention contemplates microparticles formed by spray-drying a composition comprising fibrinogen or thrombin with a nucleic acid. Preferably, these microparticles are soluble and the selected protein (i.e., fibrinogen or thrombin) creates the walls of the microparticles. Consequently, the nucleic acids are incorporated within, and between, the protein walls of the microparticle. Heath et al., Microparticles And Their Use In Wound Therapy. U.S. Pat. No. 6,113,948 (herein incorporated by reference). Following the application of the microparticles to living tissue, the subsequent reaction between the fibrinogen and thrombin creates a tissue sealant thereby releasing the incorporated compound into the immediate surrounding area.

One having skill in the art will understand that the shape of the microspheres need not be exactly spherical; only as very small particles capable of being sprayed or spread into or onto a surgical site (i.e., either open or closed). Microparticles may be comprised of a biocompatible and/or biodegradable material selected from the group consisting of polylactide, polyglycolide and copolymers of lactide/glycolide (PLGA), hyaluronic acid, modified polysaccharides and any other well known material.

### VI. Protein Assays

A variety of techniques can be employed to measure expression levels of polypeptides and proteins in a biological sample to determine biomarker expression levels. Examples of such formats include, but are not limited to, enzyme immunoassay (EIA), radioimmunoassay (RIA), Western blot analysis and enzyme linked immunoabsorbant assay (ELISA). A skilled artisan can readily adapt known protein/antibody detection methods for use in determining protein expression levels of biomarkers.

Antibodies, or antibody fragments or derivatives, can be used in methods such as Western blots, ELISA, or immunofluorescence techniques to detect biomarker expression. Either the antibodies or proteins may be immobilized on a solid support. Suitable solid phase supports or carriers include any support capable of binding an antigen or an antibody. Well-known supports or carriers include glass, polystyrene, polypropylene, polyethylene, dextran, nylon, amylases, natural and modified celluloses, polyacrylamides, gabbros, and magnetite.

One skilled in the art will know many other suitable carriers for binding antibody or antigen, and will be able to adapt such support for use with the present disclosure. The support can then be washed with suitable buffers followed by treatment with the detectably labeled antibody. The solid phase support can then be washed with the buffer a second time to remove unbound antibody. The amount of bound label on the solid support can then be detected by conventional means.

Immunohistochemistry methods are also suitable for detecting the expression levels of biomarkers. Antibodies or antisera, including polyclonal antisera, and monoclonal antibodies specific for each marker may be used to detect expression. The antibodies can be detected by direct labeling of the antibodies themselves, for example, with radioactive labels, fluorescent labels, hapten labels such as, biotin, or an enzyme such as horseradish peroxidase or alkaline phosphatase. Alternatively, unlabeled primary antibody is used in conjunction with a labeled secondary antibody, comprising antisera, polyclonal antisera or a monoclonal antibody specific for the primary antibody. Immunohistochemistry protocols and kits are well known in the art and are commercially available.

Immunological methods for detecting and measuring complex formation as a measure of protein expression using either specific polyclonal or monoclonal antibodies are known in the art. Examples of such techniques include enzyme-linked immunosorbent assays (ELISAs), radioimmunoassays (RIAs), fluorescence-activated cell sorting (FACS) and antibody arrays. Such immunoassays typically involve the measurement of complex formation between the protein and its specific antibody. These assays and their quantitation against purified, labeled standards are well known in the art. A two-site, monoclonal-based immunoassay utilizing antibodies reactive to two non-interfering epitopes or a competitive binding assay may be employed.

Numerous labels are available and commonly known in the art. Radioisotope labels include, for example, 36S, 14C, 1251, 3H, and 131I. The antibody can be labeled with the radioisotope using the techniques known in the art. Fluorescent labels include, for example, labels such as rare earth chelates (europium chelates) or fluorescein and its derivatives, rhodamine and its derivatives, dansyl, Lissamine, phycoerythrin and Texas Red are available. The fluorescent labels can be conjugated to the antibody variant using the techniques known in the art. Fluorescence can be quantified using a fluorimeter. Various enzyme-substrate labels are available and U.S. Pat. Nos. 4,275,149, 4,318,980 provides a review of some of these. The enzyme generally catalyzes a chemical alteration of the chromogenic substrate which can be measured using various techniques. For example, the enzyme may catalyze a color change in a substrate, which can be measured spectrophotometrically. Alternatively, the enzyme may alter the fluorescence or chemiluminescence of the substrate. Techniques for quantifying a change in fluorescence are described above. The chemiluminescent substrate becomes electronically excited by a chemical reaction and may then emit light which can be measured (using a chemiluminometer, for example) or donates energy to a fluorescent acceptor. Examples of enzymatic labels include luciferases (e.g., firefly luciferase and bacterial luciferase; U.S. Pat. No. 4,737,456), luciferin, 2,3-dihydrophthalazinediones, malate dehydrogenase, urease, peroxidase such as horseradish peroxidase (HRPO), alkaline phosphatase, .beta.-galactosidase, glucoamylase, lysozyme, saccharide oxidases (e.g., glucose oxidase, galactose oxidase, and glucose-6-phosphate dehydrogenase), heterocyclic oxidases (such as uricase and xanthine oxidase), lactoperoxidase, microperoxidase, and the like. Techniques for conjugating enzymes to antibodies are described in O'Sullivan et al., Methods for the Preparation of Enzyme-Antibody Conjugates for Use in Enzyme Immunoassay, in Methods in Enzymology (Ed. J. Langone & H. Van Vunakis), Academic press, New York, 73:147-166 (1981).

A detection label may be indirectly conjugated with an antibody. The skilled artisan will be aware of various techniques for achieving this. For example, the antibody can be conjugated with biotin and any of the three broad categories of labels mentioned above can be conjugated with avidin, or vice versa. Biotin binds selectively to avidin and thus, the label can be conjugated with the antibody in this indirect manner. Alternatively, to achieve indirect conjugation of the label with the antibody, the antibody is conjugated with a small hapten (e.g., digoxin) and one of the different types of labels mentioned above is conjugated with an anti-hapten antibody (e.g., anti-digoxin antibody). The antibody need not be labeled, and the presence thereof can be detected using a labeled antibody, which binds to the antibody.

### VII. Sample Preparation

Methods may involve obtaining a sample from a subject. The methods of obtaining provided herein may include methods of biopsy such as fine needle aspiration, core needle biopsy, vacuum assisted biopsy, incisional biopsy, excisional biopsy, punch biopsy, shave biopsy or skin biopsy. The sample may be obtained from any source including but not limited to blood, serum, plasma, sweat, hair follicle, buccal tissue, tears, menses, feces, or saliva. Any medical professional such as a doctor, nurse or medical technician may obtain a biological sample for testing. Yet further, the biological sample can be obtained without the assistance of a medical professional.

A sample may include but is not limited to, tissue, cells, or biological material from cells or derived from cells of a subject. The biological sample may be a heterogeneous or homogeneous population of cells or tissues. The biological sample may be obtained using any method known to the art that can provide a sample suitable for the analytical methods described herein. The sample may be obtained by non-invasive methods including but not limited to: scraping of the skin or cervix, swabbing of the cheek, saliva collection, urine collection, feces collection, collection of menses, tears, or semen.

The sample may be obtained by methods known in the art. The samples may be obtained by biopsy. The sample may be obtained by swabbing, endoscopy, scraping, phlebotomy, or any other methods known in the art. In some cases, the sample may be obtained, stored, or transported using components of a kit of the present methods. In some cases, multiple samples, such as multiple plasma or serum samples may be obtained for diagnosis by the methods described herein. In other cases, multiple samples, such as one or more samples from one tissue type (for example ovaries or related tissues) and one or more samples from another specimen (for example serum) may be obtained for diagnosis by the methods. Samples may be obtained at different times are stored and/or analyzed by different methods. For example, a sample may be obtained and analyzed by routine staining methods or any other cytological analysis methods.

The biological sample may be obtained by a physician, nurse, or other medical professional such as a medical technician, endocrinologist, cytologist, phlebotomist, radiologist, or a pulmonologist. The medical professional may indicate the appropriate test or assay to perform on the sample. A molecular profiling business may consult on which assays or tests are most appropriately indicated. The patient or subject may obtain a biological sample for testing without the assistance of a medical professional, such as obtaining a whole blood sample, a urine sample, a fecal sample, a buccal sample, or a saliva sample.

In other cases, the sample is obtained by an invasive procedure including but not limited to: biopsy, needle aspiration, blood draw, endoscopy, or phlebotomy. The method of needle aspiration may further include fine needle aspiration, core needle biopsy, vacuum assisted biopsy, or large core biopsy. Multiple samples may be obtained by the methods herein to ensure a sufficient amount of biological material.

General methods for obtaining biological samples are also known in the art. Publications such as Ramzy, Ibrahim Clinical Cytopathology and Aspiration Biopsy 2001, which is herein incorporated by reference in its entirety, describes general methods for biopsy and cytological methods.

The molecular profiling business may obtain the biological sample from a subject directly, from a medical professional, from a third party, or from a kit provided by a molecular profiling business or a third party. In some cases, the biological sample may be obtained by the molecular profiling business after the subject, a medical professional, or a third party acquires and sends the biological sample to the molecular profiling business. The molecular profiling business may provide suitable containers, and excipients for storage and transport of the biological sample to the molecular profiling business.

A medical professional need not be involved in the initial diagnosis or sample acquisition. An individual may alternatively obtain a sample through the use of an over the counter (OTC) kit. An OTC kit may contain a means for obtaining said sample as described herein, a means for storing said sample for inspection, and instructions for proper use of the kit. In some cases, molecular profiling services are included in the price for purchase of the kit. In other cases, the molecular profiling services are billed separately. A sample suitable for use by the molecular profiling business may be any material containing tissues, cells, nucleic acids, genes, gene fragments, expression products, gene expression products, or gene expression product fragments of an individual to be tested. Methods for determining sample suitability and/or adequacy are provided.

The subject may be referred to a specialist such as an oncologist, surgeon, or endocrinologist. The specialist may likewise obtain a biological sample for testing or refer the individual to a testing center or laboratory for submission of the biological sample. In some cases the medical professional may refer the subject to a testing center or laboratory for submission of the biological sample. In other cases, the subject may provide the sample. In some cases, a molecular profiling business may obtain the sample.

### VIII. Administration of Therapeutic Compositions

The therapy provided herein may comprise administration of a combination of therapeutic agents, such as a first therapy and a second therapy. The therapies may be administered in any suitable manner known in the art. For example, the first and second treatment may be administered sequentially (at different times) or concurrently (at the same time). The first and second treatments may be administered in a separate composition. The first and second treatments may be in the same composition.

The methods also relate to compositions and methods comprising therapeutic compositions. The different therapies may be administered in one composition or in more than one composition, such as 2 compositions, 3 compositions, or 4 compositions. Various combinations of the agents may be employed.

The therapeutic agents of the disclosure may be administered by the same route of administration or by different routes of administration. The therapy may be administered intravenously, intramuscularly, subcutaneously, topically, orally, transdermally, intraperitoneally, intraorbitally, by implantation, by inhalation, intrathecally, intraventricularly, or intranasally. The antibiotic may be administered intravenously, intramuscularly, subcutaneously, topically, orally, transdermally, intraperitoneally, intraorbitally, by implantation, by inhalation, intrathecally, intraventricularly, or intranasally. The appropriate dosage may be determined based on the type of disease to be treated, severity and course of the disease, the clinical condition of the individual, the individual's clinical history and response to the treatment, and the discretion of the attending physician.

The treatments may include various “unit doses.” Unit dose is defined as containing a predetermined-quantity of the therapeutic composition. The quantity to be administered, and the particular route and formulation, is within the skill of determination of those in the clinical arts. A unit dose need not be administered as a single injection but may comprise continuous infusion over a set period of time. A unit dose may comprise a single administrable dose.

Precise amounts of the therapeutic composition also depend on the judgment of the practitioner and are peculiar to each individual. Factors affecting dose include physical and clinical state of the patient, the route of administration, the intended goal of treatment (alleviation of symptoms versus cure) and the potency, stability and toxicity of the particular therapeutic substance or other therapies a subject may be undergoing.

### IX. Pharmaceutical Compositions

The compositions or agents for use in the methods, such as therapeutic agents or biomarker modulators, may be suitably contained in a pharmaceutically acceptable carrier. The carrier is non-toxic, biocompatible and is selected so as not to detrimentally affect the biological activity of the agent. The agents may be formulated into preparations for local delivery (i.e. to a specific location of the body, such as skeletal muscle or other tissue) or systemic delivery, in solid, semi-solid, gel, liquid or gaseous forms such as tablets, capsules, powders, granules, ointments, solutions, depositories, inhalants and injections allowing for oral, parenteral or surgical administration. Local administration of the compositions by coating medical devices and the like are also contemplated.

Suitable carriers for parenteral delivery via injectable, infusion or irrigation and topical delivery include distilled water, physiological phosphate-buffered saline, normal or lactated Ringer's solutions, dextrose solution, Hank's solution, or propanediol. In addition, sterile, fixed oils may be employed as a solvent or suspending medium. For this purpose any biocompatible oil may be employed including synthetic mono- or diglycerides. In addition, fatty acids such as oleic acid find use in the preparation of injectables. The carrier and agent may be compounded as a liquid, suspension, polymerizable or non-polymerizable gel, paste or salve.

The carrier may also comprise a delivery vehicle to sustain (i.e., extend, delay or regulate) the delivery of the agent(s) or to enhance the delivery, uptake, stability or pharmacokinetics of the therapeutic agent(s). Such a delivery vehicle may include, by way of non-limiting examples, microparticles, microspheres, nanospheres or nanoparticles composed of proteins, liposomes, carbohydrates, synthetic organic compounds, inorganic compounds, polymeric or copolymeric hydrogels and polymeric micelles.

The actual dosage amount of a composition administered to a patient or subject can be determined by physical and physiological factors such as body weight, severity of condition, the type of disease being treated, previous or concurrent therapeutic interventions, idiopathy of the patient and on the route of administration. The practitioner responsible for administration will, in any event, determine the concentration of active ingredient(s) in a composition and appropriate dose(s) for the individual subject.

Solutions of pharmaceutical compositions can be prepared in water suitably mixed with a surfactant, such as hydroxypropylcellulose. Dispersions also can be prepared in glycerol, liquid polyethylene glycols, mixtures thereof and in oils. Under ordinary conditions of storage and use, these preparations contain a preservative to prevent the growth of microorganisms.

The pharmaceutical compositions may be advantageously administered in the form of injectable compositions either as liquid solutions or suspensions; solid forms suitable or solution in, or suspension in, liquid prior to injection may also be prepared. These preparations also may be emulsified. A typical composition for such purpose comprises a pharmaceutically acceptable carrier. For instance, the composition may contain 10 mg or less, 25 mg, 50 mg or up to about 100 mg of human serum albumin per milliliter of phosphate buffered saline. Other pharmaceutically acceptable carriers include aqueous solutions, non-toxic excipients, including salts, preservatives, buffers and the like.

Examples of non-aqueous solvents are propylene glycol, polyethylene glycol, vegetable oil and injectable organic esters such as ethyloleate. Aqueous carriers include water, alcoholic/aqueous solutions, saline solutions, parenteral vehicles such as sodium chloride, Ringer's dextrose, etc. Intravenous vehicles include fluid and nutrient replenishers. Preservatives include antimicrobial agents, antifungal agents, anti-oxidants, chelating agents and inert gases. The pH and exact concentration of the various components the pharmaceutical composition are adjusted according to well-known parameters.

Additional formulations are suitable for oral administration. Oral formulations include such typical excipients as, for example, pharmaceutical grades of mannitol, lactose, starch, magnesium stearate, sodium saccharine, cellulose, magnesium carbonate and the like. The compositions take the form of solutions, suspensions, tablets, pills, capsules, sustained release formulations or powders.

The pharmaceutical compositions may include classic pharmaceutical preparations. Administration of pharmaceutical compositions may be via any common route so long as the target tissue is available via that route. This may include oral, nasal, buccal, rectal, vaginal or topical. Alternatively, administration may be by orthotopic, intradermal, subcutaneous, intramuscular, intraperitoneal or intravenous injection. Such compositions would normally be administered as pharmaceutically acceptable compositions that include physiologically acceptable carriers, buffers or other excipients. For treatment of conditions of the lungs, aerosol delivery can be used. Volume of the aerosol is between about 0.01 ml and 0.5 ml.

An effective amount of the pharmaceutical composition is determined based on the intended goal. The term “unit dose” or “dosage” refers to physically discrete units suitable for use in a subject, each unit containing a predetermined-quantity of the pharmaceutical composition calculated to produce the desired responses discussed above in association with its administration, i.e., the appropriate route and treatment regimen. The quantity to be administered, both according to number of treatments and unit dose, depends on the protection or effect desired.

Precise amounts of the pharmaceutical composition also depend on the judgment of the practitioner and are peculiar to each individual. Factors affecting the dose include the physical and clinical state of the patient, the route of administration, the intended goal of treatment (e.g., alleviation of symptoms versus cure) and the potency, stability and toxicity of the particular therapeutic substance.

### X. Tables

TABLE S1
Differentially Expressed Plasma miRNAs in Ccm1 Homozygous
vs. Wild Type Mice (p < 0.1, FDR-corrected)

| | baseMean | log2FoldChange | IfcSE | stat | pvalue | padj |
|---|---|---|---|---|---|---|
| mmu-miR-124-3p | 85.953 | −5.772 | 1.343 | −4.299 | 0.000 | 0.010 |
| mmu-miR-466h-3p | 4.553 | −3.802 | 1.044 | −3.641 | 0.000 | 0.081 |
| mmu-miR-127-3p | 10787.541 | 0.851 | 0.272 | 3.126 | 0.002 | 0.097 |
| mmu-miR-127-5p | 51.125 | 1.262 | 0.377 | 3.353 | 0.001 | 0.097 |
| mmu-miR-128-3p | 13352.037 | −0.952 | 0.305 | −3.123 | 0.002 | 0.097 |
| mmu-miR-133a-3p | 11892.289 | 1.292 | 0.412 | 3.132 | 0.002 | 0.097 |
| mmu-miR-133b-3p | 205.775 | 1.318 | 0.429 | 3.075 | 0.002 | 0.097 |
| mmu-miR-182-5p | 5295.586 | 0.883 | 0.286 | 3.093 | 0.002 | 0.097 |
| mmu-miR-194-1-3p | 6.874 | −2.854 | 0.833 | −3.426 | 0.001 | 0.097 |
| mmu-miR-411-5p | 356.455 | 1.002 | 0.316 | 3.174 | 0.002 | 0.097 |
| mmu-miR-431-5p | 99.831 | 1.115 | 0.357 | 3.118 | 0.002 | 0.097 |
| mmu-miR-541-5p | 956.632 | 0.853 | 0.260 | 3.275 | 0.001 | 0.097 |
| mmu-miR-9-5p | 66.205 | −2.870 | 0.928 | −3.093 | 0.002 | 0.097 |
| mmu-miR-133a-5p | 30.314 | 1.480 | 0.493 | 3.004 | 0.003 | 0.098 |
| mmu-miR-203-3p | 2351.952 | 0.814 | 0.270 | 3.014 | 0.003 | 0.098 |
| mmu-miR-337-5p | 37.438 | 1.386 | 0.461 | 3.007 | 0.003 | 0.098 |
| mmu-miR-434-5p | 334.468 | 0.977 | 0.327 | 2.989 | 0.003 | 0.098 |

TABLE S2
Differentially Expressed Plasma miRNAs in Ccm1 Heterozygous
vs. Wild Type Mice (p < 0.1, FDR-corrected)

| | baseMean | log2Fold Change | IfcSE | stat | pvalue | padj |
|---|---|---|---|---|---|---|
| mmu-miR-351-5p | 473.644 | 1.488 | 0.376 | 3.962 | 0.000 | 0.011 |
| mmu-miR-486a-5p | 1407237.484 | −0.994 | 0.257 | −3.864 | 0.000 | 0.011 |
| mmu-miR-486b-5p | 1394212.349 | −1.001 | 0.257 | −3.895 | 0.000 | 0.011 |
| mmu-miR-8116 | 12.165 | 1.894 | 0.487 | 3.889 | 0.000 | 0.011 |
| mmu-miR-223-5p | 43.269 | 1.412 | 0.380 | 3.715 | 0.000 | 0.015 |
| mmu-miR-486b-3p | 252.264 | −0.748 | 0.203 | −3.686 | 0.000 | 0.015 |
| mmu-miR-5100 | 16.635 | −2.325 | 0.673 | −3.453 | 0.001 | 0.031 |
| mmu-miR-25-3p | 23890.353 | −0.624 | 0.191 | −3.267 | 0.001 | 0.033 |
| mmu-miR-330-5p | 19.434 | 1.507 | 0.457 | 3.298 | 0.001 | 0.033 |
| mmu-miR-375-3p | 3680.210 | 1.442 | 0.426 | 3.388 | 0.001 | 0.033 |
| mmu-miR-425-3p | 54.322 | 1.318 | 0.395 | 3.339 | 0.001 | 0.033 |
| mmu-miR-669a-5p | 43.453 | 0.934 | 0.284 | 3.289 | 0.001 | 0.033 |
| mmu-miR-669p-5p | 43.453 | 0.934 | 0.284 | 3.289 | 0.001 | 0.033 |
| mmu-miR-211-5p | 4.346 | −3.509 | 1.101 | −3.187 | 0.001 | 0.041 |
| mmu-miR-15b-5p | 213.837 | −0.572 | 0.183 | −3.128 | 0.002 | 0.045 |
| mmu-miR-1983 | 7.401 | 1.705 | 0.549 | 3.104 | 0.002 | 0.045 |
| mmu-miR-486a-3p | 1671.109 | −0.611 | 0.196 | −3.117 | 0.002 | 0.045 |
| mmu-miR-15a-3p | 32.273 | −1.103 | 0.366 | −3.016 | 0.003 | 0.049 |
| mmu-miR-301b-3p | 28.101 | 1.268 | 0.417 | 3.041 | 0.002 | 0.049 |
| mmu-miR-330-3p | 30.784 | 1.433 | 0.469 | 3.054 | 0.002 | 0.049 |
| mmu-miR-451a | 17623.958 | −0.912 | 0.302 | −3.025 | 0.002 | 0.049 |
| mmu-miR-130a-3p | 2337.462 | 0.595 | 0.204 | 2.919 | 0.004 | 0.061 |
| mmu-miR-144-3p | 679.086 | −0.882 | 0.306 | −2.881 | 0.004 | 0.061 |
| mmu-miR-144-5p | 273.502 | −0.810 | 0.280 | −2.897 | 0.004 | 0.061 |
| mmu-miR-326-3p | 125.499 | 0.631 | 0.218 | 2.889 | 0.004 | 0.061 |
| mmu-miR-350-3p | 19.486 | −1.182 | 0.405 | −2.915 | 0.004 | 0.061 |
| mmu-miR-106b-5p | 371.225 | −0.784 | 0.276 | −2.840 | 0.005 | 0.063 |
| mmu-miR-16-2-3p | 61.036 | −0.866 | 0.306 | −2.829 | 0.005 | 0.063 |
| mmu-miR-191-5p | 58966.629 | 0.844 | 0.296 | 2.848 | 0.004 | 0.063 |
| mmu-miR-8114 | 120.380 | −0.714 | 0.253 | −2.824 | 0.005 | 0.063 |
| mmu-miR-181b-5p | 924.718 | 0.613 | 0.222 | 2.766 | 0.006 | 0.068 |
| mmu-miR-1843a-5p | 239.894 | 0.681 | 0.245 | 2.774 | 0.006 | 0.068 |
| mmu-miR-335-3p | 14.560 | 1.385 | 0.501 | 2.767 | 0.006 | 0.068 |
| mmu-miR-93-5p | 5619.894 | −0.580 | 0.211 | −2.743 | 0.006 | 0.071 |
| mmu-miR-361-3p | 74.506 | 0.754 | 0.284 | 2.656 | 0.008 | 0.090 |
| mmu-miR-30c-5p | 1621.145 | 0.823 | 0.311 | 2.646 | 0.008 | 0.090 |
| mmu-miR-181d-5p | 684.172 | 0.745 | 0.283 | 2.626 | 0.009 | 0.093 |
| mmu-miR-15a-5p | 1573.689 | −0.638 | 0.245 | −2.600 | 0.009 | 0.098 |
| mmu-miR-20b-5p | 3.746 | 2.383 | 0.921 | 2.587 | 0.010 | 0.099 |
| mmu-miR-92b-3p | 328.922 | 0.741 | 0.287 | 2.579 | 0.010 | 0.099 |
| mmu-miR-142a-5p | 24096.350 | −0.405 | 0.158 | −2.567 | 0.010 | 0.099 |
| mmu-miR-214-5p | 14.956 | −1.281 | 0.503 | −2.545 | 0.011 | 0.100 |
| mmu-miR-30e-3p | 85.675 | 0.553 | 0.217 | 2.553 | 0.011 | 0.100 |
| mmu-miR-450a-5p | 6.924 | 1.468 | 0.578 | 2.541 | 0.011 | 0.100 |

TABLE S3
Differentially Expressed Plasma miRNAs in Ccm3 Homozygous
vs. Wild Type Mice (p < 0.1, FDR-corrected)

| | baseMean | log2FoldChange | IfcSE | stat | pvalue | padj |
|---|---|---|---|---|---|---|
| mmu-miR-139-5p | 1185.517 | 1.263 | 0.190 | 6.639 | 0.000 | 0.000 |
| mmu-miR-193b-3p | 380.706 | 1.670 | 0.278 | 6.009 | 0.000 | 0.000 |
| mmu-miR-6395 | 378.468 | 1.863 | 0.349 | 5.336 | 0.000 | 0.000 |
| mmu-miR-212-3p | 130.057 | 1.331 | 0.263 | 5.054 | 0.000 | 0.000 |
| mmu-miR-541-5p | 956.632 | 1.459 | 0.287 | 5.088 | 0.000 | 0.000 |
| mmu-miR-182-5p | 5295.586 | 1.531 | 0.315 | 4.868 | 0.000 | 0.000 |
| mmu-miR-127-3p | 10787.541 | 1.425 | 0.300 | 4.756 | 0.000 | 0.000 |
| mmu-miR-410-3p | 353.619 | 1.678 | 0.353 | 4.751 | 0.000 | 0.000 |
| mmu-miR-181c-5p | 12259.653 | −0.870 | 0.185 | −4.693 | 0.000 | 0.000 |
| mmu-miR-140-3p | 45481.352 | −0.751 | 0.166 | −4.537 | 0.000 | 0.000 |
| mmu-miR-16-2-3p | 622.534 | −1.641 | 0.361 | −4.547 | 0.000 | 0.000 |
| mmu-miR-144-5p | 2165.267 | −2.315 | 0.512 | −4.518 | 0.000 | 0.000 |
| mmu-miR-142a-3p | 2480.245 | −1.460 | 0.329 | −4.446 | 0.000 | 0.000 |
| mmu-let-7f-5p | 21527.948 | −1.625 | 0.382 | −4.248 | 0.000 | 0.001 |
| mmu-miR-451a | 159170.182 | −1.514 | 0.356 | −4.257 | 0.000 | 0.001 |
| mmu-miR-455-5p | 174.426 | −2.221 | 0.526 | −4.227 | 0.000 | 0.001 |
| mmu-miR-341-3p | 93.713 | 2.089 | 0.497 | 4.206 | 0.000 | 0.001 |
| mmu-miR-15a-5p | 11865.305 | −1.584 | 0.380 | −4.172 | 0.000 | 0.001 |
| mmu-miR-183-5p | 470.826 | 1.474 | 0.356 | 4.140 | 0.000 | 0.001 |
| mmu-miR-196a-5p | 33.968 | −3.058 | 0.736 | −4.156 | 0.000 | 0.001 |
| mmu-miR-296-3p | 61.881 | 1.333 | 0.322 | 4.139 | 0.000 | 0.001 |
| mmu-miR-666-5p | 15.782 | 2.498 | 0.597 | 4.182 | 0.000 | 0.001 |
| mmu-miR-133b-3p | 205.775 | 1.926 | 0.471 | 4.086 | 0.000 | 0.001 |
| mmu-miR-874-3p | 46.758 | 1.482 | 0.363 | 4.083 | 0.000 | 0.001 |
| mmu-let-7i-5p | 47380.180 | −1.186 | 0.294 | −4.034 | 0.000 | 0.001 |
| mmu-miR-140-5p | 402.785 | −1.080 | 0.271 | −3.988 | 0.000 | 0.002 |
| mmu-miR-16-1-3p | 110.459 | −1.883 | 0.475 | −3.962 | 0.000 | 0.002 |
| mmu-miR-26b-5p | 6704.618 | −1.503 | 0.379 | −3.967 | 0.000 | 0.002 |
| mmu-miR-365-3p | 168.827 | 1.424 | 0.360 | 3.961 | 0.000 | 0.002 |
| mmu-miR-99b-5p | 4584.465 | 1.236 | 0.310 | 3.982 | 0.000 | 0.002 |
| mmu-miR-15b-5p | 1552.196 | −1.456 | 0.373 | −3.897 | 0.000 | 0.002 |
| mmu-miR-487b-3p | 41.749 | 2.742 | 0.704 | 3.894 | 0.000 | 0.002 |
| mmu-miR-133a-3p | 11892.289 | 1.754 | 0.454 | 3.860 | 0.000 | 0.002 |
| mmu-miR-340-5p | 1062.660 | −1.117 | 0.292 | −3.827 | 0.000 | 0.002 |
| mmu-miR-195a-5p | 530.598 | −1.311 | 0.345 | −3.801 | 0.000 | 0.003 |
| mmu-miR-434-3p | 3851.695 | 1.624 | 0.427 | 3.807 | 0.000 | 0.003 |
| mmu-miR-101b-3p | 2968.919 | −1.323 | 0.349 | −3.794 | 0.000 | 0.003 |
| mmu-miR-344d-3p | 13.997 | 3.592 | 0.951 | 3.779 | 0.000 | 0.003 |
| mmu-miR-298-5p | 949.802 | 0.843 | 0.224 | 3.765 | 0.000 | 0.003 |
| mmu-miR-380-3p | 18.215 | 2.368 | 0.631 | 3.755 | 0.000 | 0.003 |
| mmu-miR-323-3p | 14.998 | 3.051 | 0.815 | 3.744 | 0.000 | 0.003 |
| mmu-miR-125b-1-3p | 230.220 | 1.054 | 0.282 | 3.733 | 0.000 | 0.003 |
| mmu-miR-154-5p | 16.220 | 1.975 | 0.532 | 3.710 | 0.000 | 0.003 |
| mmu-miR-340-3p | 30.748 | −1.053 | 0.288 | −3.659 | 0.000 | 0.004 |
| mmu-miR-433-3p | 45.341 | 2.185 | 0.597 | 3.662 | 0.000 | 0.004 |
| mmu-miR-106b-5p | 3073.319 | −1.528 | 0.421 | −3.625 | 0.000 | 0.004 |
| mmu-miR-138-1-3p | 27.826 | 1.890 | 0.522 | 3.624 | 0.000 | 0.004 |
| mmu-miR-1983 | 35.309 | 1.901 | 0.522 | 3.639 | 0.000 | 0.004 |
| mmu-miR-210-3p | 2475.284 | 0.768 | 0.212 | 3.627 | 0.000 | 0.004 |
| mmu-miR-144-3p | 6407.334 | −1.692 | 0.471 | −3.592 | 0.000 | 0.004 |
| mmu-miR-5113 | 113.864 | −0.954 | 0.267 | −3.577 | 0.000 | 0.004 |
| mmu-miR-300-3p | 374.295 | 1.223 | 0.343 | 3.561 | 0.000 | 0.005 |
| mmu-miR-181c-3p | 393.571 | −0.726 | 0.205 | −3.546 | 0.000 | 0.005 |
| mmu-miR-148a-5p | 346.196 | 1.123 | 0.317 | 3.540 | 0.000 | 0.005 |
| mmu-miR-322-5p | 383.639 | −1.324 | 0.378 | −3.505 | 0.000 | 0.005 |
| mmu-let-7d-5p | 5852.546 | −1.263 | 0.362 | −3.486 | 0.000 | 0.006 |
| mmu-miR-374b-5p | 396.776 | −1.315 | 0.378 | −3.482 | 0.000 | 0.006 |
| mmu-miR-129-5p | 18.532 | 2.517 | 0.725 | 3.473 | 0.001 | 0.006 |
| mmu-miR-15b-3p | 672.543 | −1.354 | 0.391 | −3.460 | 0.001 | 0.006 |
| mmu-miR-503-5p | 12.566 | 1.328 | 0.391 | 3.396 | 0.001 | 0.007 |
| mmu-miR-350-3p | 124.215 | −1.150 | 0.339 | −3.389 | 0.001 | 0.007 |
| mmu-miR-193b-5p | 8.593 | 2.779 | 0.826 | 3.366 | 0.001 | 0.008 |
| mmu-miR-129b-5p | 8.036 | 3.025 | 0.908 | 3.331 | 0.001 | 0.009 |
| mmu-miR-210-5p | 82.232 | 0.989 | 0.297 | 3.333 | 0.001 | 0.009 |
| mmu-miR-676-5p | 158.067 | −0.773 | 0.233 | −3.321 | 0.001 | 0.009 |
| mmu-miR-132-3p | 657.627 | 0.990 | 0.301 | 3.293 | 0.001 | 0.010 |
| mmu-miR-26a-5p | 39052.438 | −0.782 | 0.238 | −3.290 | 0.001 | 0.010 |
| mmu-miR-2137 | 4.491 | 3.873 | 1.180 | 3.282 | 0.001 | 0.010 |
| mmu-miR-16-5p | 407517.707 | −0.967 | 0.297 | −3.257 | 0.001 | 0.011 |
| mmu-miR-375-3p | 9377.323 | 1.820 | 0.561 | 3.243 | 0.001 | 0.011 |
| mmu-miR-667-3p | 30.749 | 1.984 | 0.614 | 3.230 | 0.001 | 0.011 |
| mmu-miR-1966-3p | 2.265 | 3.456 | 1.075 | 3.214 | 0.001 | 0.012 |
| mmu-miR-3535 | 25.654 | 1.887 | 0.592 | 3.189 | 0.001 | 0.013 |
| mmu-miR-376b-5p | 14.869 | 1.418 | 0.449 | 3.158 | 0.002 | 0.014 |
| mmu-let-7a-5p | 8292.167 | −0.756 | 0.240 | −3.152 | 0.002 | 0.014 |
| mmu-miR-15a-3p | 308.038 | −1.326 | 0.424 | −3.127 | 0.002 | 0.015 |
| mmu-miR-181d-5p | 2090.798 | −0.646 | 0.208 | −3.114 | 0.002 | 0.015 |
| mmu-miR-382-5p | 31.920 | 1.698 | 0.545 | 3.116 | 0.002 | 0.015 |
| mmu-miR-93-5p | 44525.520 | −0.938 | 0.303 | −3.096 | 0.002 | 0.016 |
| mmu-miR-409-3p | 223.245 | 1.173 | 0.383 | 3.065 | 0.002 | 0.018 |
| mmu-miR-666-3p | 17.248 | 2.034 | 0.668 | 3.045 | 0.002 | 0.019 |
| mmu-miR-1950 | 3.858 | −3.364 | 1.126 | −2.988 | 0.003 | 0.022 |
| mmu-miR-98-5p | 813.779 | −1.085 | 0.363 | −2.991 | 0.003 | 0.022 |
| mmu-miR-30e-5p | 82526.392 | −0.712 | 0.239 | −2.979 | 0.003 | 0.022 |
| mmu-miR-5100 | 22.754 | −1.363 | 0.463 | −2.946 | 0.003 | 0.025 |
| mmu-miR-431-3p | 13.368 | 2.559 | 0.876 | 2.922 | 0.003 | 0.026 |
| mmu-miR-138-5p | 606.815 | 2.259 | 0.801 | 2.818 | 0.005 | 0.036 |
| mmu-miR-378a-3p | 23691.629 | 0.728 | 0.259 | 2.814 | 0.005 | 0.036 |
| mmu-miR-1247-3p | 3.535 | 2.563 | 0.915 | 2.801 | 0.005 | 0.037 |
| mmu-miR-127-5p | 51.125 | 1.159 | 0.415 | 2.790 | 0.005 | 0.038 |
| mmu-miR-505-3p | 10.199 | −1.755 | 0.631 | −2.782 | 0.005 | 0.039 |
| mmu-miR-714 | 14.199 | 1.830 | 0.661 | 2.769 | 0.006 | 0.040 |
| mmu-miR-542-3p | 44.798 | 0.868 | 0.316 | 2.747 | 0.006 | 0.042 |
| mmu-miR-96-5p | 3.469 | 3.671 | 1.339 | 2.740 | 0.006 | 0.042 |
| mmu-miR-540-5p | 14.496 | 1.245 | 0.458 | 2.720 | 0.007 | 0.045 |
| mmu-miR-142a-5p | 131312.401 | −0.711 | 0.263 | −2.701 | 0.007 | 0.047 |
| mmu-miR-423-3p | 9666.129 | 0.705 | 0.262 | 2.691 | 0.007 | 0.048 |
| mmu-miR-6240 | 30.483 | 1.112 | 0.414 | 2.686 | 0.007 | 0.048 |
| mmu-miR-196b-5p | 48.503 | −1.290 | 0.482 | −2.675 | 0.007 | 0.049 |
| mmu-miR-32-5p | 73.096 | −1.053 | 0.394 | −2.670 | 0.008 | 0.049 |
| mmu-miR-6970-5p | 3.278 | 2.855 | 1.074 | 2.658 | 0.008 | 0.051 |
| mmu-miR-455-3p | 27.593 | −1.663 | 0.627 | −2.651 | 0.008 | 0.051 |
| mmu-miR-17-3p | 581.300 | −0.953 | 0.361 | −2.639 | 0.008 | 0.052 |
| mmu-miR-30c-2-3p | 61.898 | 0.592 | 0.225 | 2.635 | 0.008 | 0.053 |
| mmu-miR-194-5p | 330.592 | −0.869 | 0.330 | −2.629 | 0.009 | 0.053 |
| mmu-miR-383-5p | 15.716 | 3.059 | 1.164 | 2.627 | 0.009 | 0.053 |
| mmu-miR-186-5p | 50731.636 | −0.672 | 0.257 | −2.614 | 0.009 | 0.054 |
| mmu-miR-23a-3p | 6753.922 | 0.474 | 0.182 | 2.604 | 0.009 | 0.055 |
| mmu-miR-467d-5p | 17.538 | −1.337 | 0.519 | −2.578 | 0.010 | 0.059 |
| mmu-miR-369-5p | 46.892 | 1.449 | 0.563 | 2.574 | 0.010 | 0.059 |
| mmu-miR-668-3p | 32.584 | 1.450 | 0.565 | 2.564 | 0.010 | 0.061 |
| mmu-miR-497a-5p | 760.861 | −1.481 | 0.582 | −2.546 | 0.011 | 0.063 |
| mmu-miR-3084-3p | 7.346 | 1.448 | 0.570 | 2.542 | 0.011 | 0.063 |
| mmu-miR-466b-3p | 5.595 | −1.936 | 0.764 | −2.534 | 0.011 | 0.063 |
| mmu-miR-466c-3p | 5.595 | −1.936 | 0.764 | −2.534 | 0.011 | 0.063 |
| mmu-miR-466e-3p | 5.595 | −1.936 | 0.764 | −2.534 | 0.011 | 0.063 |
| mmu-miR-181a-1-3p | 137.655 | −0.635 | 0.253 | −2.513 | 0.012 | 0.067 |
| mmu-miR-148a-3p | 18616.590 | 0.888 | 0.355 | 2.501 | 0.012 | 0.067 |
| mmu-miR-3072-3p | 3.361 | 2.554 | 1.021 | 2.502 | 0.012 | 0.067 |
| mmu-miR-425-5p | 5359.627 | −0.371 | 0.148 | −2.507 | 0.012 | 0.067 |
| mmu-let-7g-5p | 22072.257 | −0.704 | 0.283 | −2.488 | 0.013 | 0.069 |
| mmu-miR-679-5p | 5.475 | 2.096 | 0.846 | 2.478 | 0.013 | 0.070 |
| mmu-miR-20a-5p | 611.512 | −0.855 | 0.346 | −2.472 | 0.013 | 0.070 |
| mmu-miR-466p-3p | 5.466 | −1.882 | 0.762 | −2.469 | 0.014 | 0.070 |
| mmu-miR-483-3p | 5.653 | 2.395 | 0.969 | 2.472 | 0.013 | 0.070 |
| mmu-miR-7a-1-3p | 102.419 | −0.809 | 0.328 | −2.465 | 0.014 | 0.071 |
| mmu-miR-543-3p | 3.787 | 2.326 | 0.951 | 2.446 | 0.014 | 0.074 |
| mmu-miR-488-3p | 12.445 | 3.176 | 1.303 | 2.438 | 0.015 | 0.075 |
| mmu-miR-30d-3p | 88.197 | −0.728 | 0.302 | −2.414 | 0.016 | 0.079 |
| mmu-miR-134-5p | 54.804 | 1.071 | 0.445 | 2.407 | 0.016 | 0.080 |
| mmu-miR-9769-3p | 15.760 | −0.989 | 0.411 | −2.404 | 0.016 | 0.080 |
| mmu-miR-582-3p | 89.037 | 0.786 | 0.330 | 2.384 | 0.017 | 0.084 |
| mmu-miR-6691-5p | 1.487 | −3.435 | 1.450 | −2.368 | 0.018 | 0.087 |
| mmu-miR-466a-3p | 5.415 | −1.861 | 0.787 | −2.363 | 0.018 | 0.088 |
| mmu-miR-33-5p | 36.681 | −1.031 | 0.439 | −2.348 | 0.019 | 0.091 |
| mmu-miR-181b-5p | 4408.986 | −0.854 | 0.368 | −2.320 | 0.020 | 0.096 |
| mmu-miR-18a-5p | 171.335 | −0.823 | 0.354 | −2.321 | 0.020 | 0.096 |
| mmu-miR-345-5p | 170.691 | −0.530 | 0.229 | −2.312 | 0.021 | 0.098 |
| mmu-miR-370-3p | 20.562 | 1.453 | 0.630 | 2.305 | 0.021 | 0.098 |
| mmu-miR-421-3p | 5472.005 | −0.581 | 0.252 | −2.303 | 0.021 | 0.098 |
| mmu-miR-7653-5p | 25.269 | −0.963 | 0.417 | −2.307 | 0.021 | 0.098 |
| mmu-miR-7a-2-3p | 3.510 | 2.349 | 1.018 | 2.307 | 0.021 | 0.098 |

TABLE S4
Differentially Expressed Plasma miRNAs in Ccm3 Heterozygous
vs. Wild Type Mice (p < 0.1, FDR-corrected)

| | baseMean | log2FoldChange | IfcSE | stat | pvalue | padj |
|---|---|---|---|---|---|---|
| mmu-miR-351-5p | 473.644 | 1.574 | 0.376 | 4.192 | 0.000 | 0.009 |
| mmu-miR-450a-5p | 6.924 | 2.135 | 0.564 | 3.783 | 0.000 | 0.024 |
| mmu-miR-669a-5p | 43.453 | 1.007 | 0.284 | 3.543 | 0.000 | 0.031 |
| mmu-miR-669p-5p | 43.453 | 1.007 | 0.284 | 3.543 | 0.000 | 0.031 |
| mmu-miR-223-3p | 799.411 | 0.468 | 0.136 | 3.442 | 0.001 | 0.036 |
| mmu-miR-191-5p | 58966.629 | 0.890 | 0.296 | 3.004 | 0.003 | 0.099 |
| mmu-miR-1983 | 7.401 | 1.655 | 0.550 | 3.010 | 0.003 | 0.099 |
| mmu-miR-24-2-5p | 260.188 | 0.515 | 0.173 | 2.984 | 0.003 | 0.099 |
| mmu-miR-30c-5p | 1621.145 | 0.954 | 0.311 | 3.068 | 0.002 | 0.099 |

TABLE S5
Differentially Expressed Plasma miRNAs in Ccm1 Homozygous
vs. Ccm3 Homozygous Mice (p < 0.1, FDR-corrected)

| | baseMean | log2Fold Change | IfcSE | stat | pvalue | padj |
|---|---|---|---|---|---|---|
| mmu-miR-139-5p | 1185.517 | 1.252 | 0.195 | 6.408 | 0.000 | 0.000 |
| mmu-miR-124-3p | 85.953 | 8.709 | 1.479 | 5.888 | 0.000 | 0.000 |
| mmu-miR-6395 | 378.468 | 2.013 | 0.359 | 5.611 | 0.000 | 0.000 |
| mmu-miR-344d-3p | 13.997 | 5.548 | 1.073 | 5.169 | 0.000 | 0.000 |
| mmu-miR-195a-5p | 530.598 | −1.792 | 0.354 | −5.063 | 0.000 | 0.000 |
| mmu-miR-212-3p | 130.057 | 1.359 | 0.270 | 5.023 | 0.000 | 0.000 |
| mmu-miR-196a-5p | 33.968 | −3.563 | 0.750 | −4.749 | 0.000 | 0.000 |
| mmu-miR-129-5p | 18.532 | 3.593 | 0.766 | 4.688 | 0.000 | 0.000 |
| mmu-miR-9-5p | 66.205 | 4.818 | 1.042 | 4.625 | 0.000 | 0.000 |
| mmu-miR-193b-3p | 380.706 | 1.310 | 0.285 | 4.598 | 0.000 | 0.000 |
| mmu-miR-455-5p | 174.426 | −2.437 | 0.539 | −4.521 | 0.000 | 0.000 |
| mmu-miR-330-3p | 115.566 | 1.803 | 0.413 | 4.370 | 0.000 | 0.001 |
| mmu-miR-140-3p | 45481.352 | −0.731 | 0.170 | −4.299 | 0.000 | 0.001 |
| mmu-miR-138-1-3p | 27.826 | 2.299 | 0.540 | 4.254 | 0.000 | 0.001 |
| mmu-miR-503-5p | 12.566 | 1.749 | 0.412 | 4.243 | 0.000 | 0.001 |
| mmu-miR-383-5p | 15.716 | 5.256 | 1.262 | 4.165 | 0.000 | 0.001 |
| mmu-miR-7a-2-3p | 3.510 | 5.110 | 1.245 | 4.104 | 0.000 | 0.002 |
| mmu-miR-672-5p | 88.954 | 2.107 | 0.521 | 4.040 | 0.000 | 0.002 |
| mmu-miR-219a-2-3p | 2.920 | 4.711 | 1.183 | 3.980 | 0.000 | 0.002 |
| mmu-miR-451a | 159170.182 | −1.437 | 0.365 | −3.934 | 0.000 | 0.003 |
| mmu-miR-874-3p | 46.758 | 1.451 | 0.372 | 3.897 | 0.000 | 0.003 |
| mmu-miR-129-2-3p | 31.699 | 4.141 | 1.066 | 3.884 | 0.000 | 0.003 |
| mmu-miR-181c-5p | 12259.653 | −0.731 | 0.190 | −3.840 | 0.000 | 0.003 |
| mmu-miR-423-3p | 9666.129 | 1.020 | 0.269 | 3.791 | 0.000 | 0.004 |
| mmu-miR-296-3p | 61.881 | 1.230 | 0.330 | 3.730 | 0.000 | 0.005 |
| mmu-miR-140-5p | 402.785 | −1.025 | 0.278 | −3.685 | 0.000 | 0.006 |
| mmu-miR-34c-5p | 113.551 | −1.751 | 0.483 | −3.622 | 0.000 | 0.007 |
| mmu-miR-877-3p | 43.733 | 1.229 | 0.343 | 3.584 | 0.000 | 0.008 |
| mmu-miR-138-5p | 606.815 | 2.937 | 0.823 | 3.567 | 0.000 | 0.008 |
| mmu-miR-144-5p | 2165.267 | −1.876 | 0.526 | −3.565 | 0.000 | 0.008 |
| mmu-miR-132-3p | 657.627 | 1.093 | 0.309 | 3.539 | 0.000 | 0.008 |
| mmu-miR-15a-5p | 11865.305 | −1.373 | 0.390 | −3.520 | 0.000 | 0.008 |
| mmu-miR-26b-5p | 6704.618 | −1.371 | 0.389 | −3.523 | 0.000 | 0.008 |
| mmu-miR-350-3p | 124.215 | −1.221 | 0.348 | −3.513 | 0.000 | 0.008 |
| mmu-miR-16-2-3p | 622.534 | −1.290 | 0.371 | −3.480 | 0.001 | 0.009 |
| mmu-miR-375-3p | 9377.323 | 2.003 | 0.576 | 3.475 | 0.001 | 0.009 |
| mmu-miR-455-3p | 27.593 | −2.221 | 0.640 | −3.473 | 0.001 | 0.009 |
| mmu-let-7f-5p | 21527.948 | −1.354 | 0.393 | −3.447 | 0.001 | 0.010 |
| mmu-miR-99b-5p | 4584.465 | 1.094 | 0.319 | 3.429 | 0.001 | 0.010 |
| mmu-miR-210-3p | 2475.284 | 0.742 | 0.218 | 3.412 | 0.001 | 0.010 |
| mmu-miR-210-5p | 82.232 | 1.029 | 0.305 | 3.376 | 0.001 | 0.012 |
| mmu-miR-666-3p | 17.248 | 2.327 | 0.690 | 3.370 | 0.001 | 0.012 |
| mmu-miR-497a-5p | 760.861 | −1.991 | 0.597 | −3.333 | 0.001 | 0.013 |
| mmu-miR-1983 | 35.309 | 1.755 | 0.535 | 3.280 | 0.001 | 0.015 |
| mmu-miR-323-3p | 14.998 | 2.728 | 0.830 | 3.286 | 0.001 | 0.015 |
| mmu-let-7i-5p | 47380.180 | −0.976 | 0.302 | −3.234 | 0.001 | 0.017 |
| mmu-miR-15b-5p | 1552.196 | −1.237 | 0.384 | −3.224 | 0.001 | 0.017 |
| mmu-miR-214-5p | 144.733 | −1.289 | 0.402 | −3.207 | 0.001 | 0.018 |
| mmu-miR-6948-3p | 19.021 | 1.607 | 0.503 | 3.193 | 0.001 | 0.019 |
| mmu-miR-340-3p | 30.748 | −0.937 | 0.294 | −3.184 | 0.001 | 0.019 |
| mmu-miR-298-5p | 949.802 | 0.726 | 0.230 | 3.159 | 0.002 | 0.020 |
| mmu-miR-487b-3p | 41.749 | 2.262 | 0.720 | 3.143 | 0.002 | 0.021 |
| mmu-miR-341-3p | 93.713 | 1.576 | 0.508 | 3.099 | 0.002 | 0.024 |
| mmu-miR-138-2-3p | 5.578 | 2.861 | 0.930 | 3.077 | 0.002 | 0.025 |
| mmu-miR-433-3p | 45.341 | 1.879 | 0.611 | 3.076 | 0.002 | 0.025 |
| mmu-miR-199a-3p | 10476.790 | −1.025 | 0.336 | −3.052 | 0.002 | 0.026 |
| mmu-miR-199b-3p | 10467.979 | −1.024 | 0.336 | −3.050 | 0.002 | 0.026 |
| mmu-miR-466h-3p | 4.553 | 3.444 | 1.137 | 3.029 | 0.002 | 0.028 |
| mmu-miR-431-3p | 13.368 | 2.724 | 0.902 | 3.019 | 0.003 | 0.028 |
| mmu-miR-702-3p | 18.931 | 1.395 | 0.472 | 2.954 | 0.003 | 0.034 |
| mmu-miR-142a-3p | 2480.245 | −0.987 | 0.337 | −2.925 | 0.003 | 0.035 |
| mmu-miR-16-1-3p | 110.459 | −1.432 | 0.488 | −2.931 | 0.003 | 0.035 |
| mmu-miR-16-5p | 407517.707 | −0.888 | 0.305 | −2.914 | 0.004 | 0.035 |
| mmu-miR-488-3p | 12.445 | 3.962 | 1.356 | 2.923 | 0.003 | 0.035 |
| mmu-miR-760-3p | 3.824 | 3.387 | 1.155 | 2.934 | 0.003 | 0.035 |
| mmu-miR-935 | 3.480 | 3.828 | 1.312 | 2.918 | 0.004 | 0.035 |
| mmu-miR-322-5p | 383.639 | −1.126 | 0.388 | −2.902 | 0.004 | 0.036 |
| mmu-miR-5113 | 113.864 | −0.791 | 0.274 | −2.891 | 0.004 | 0.037 |
| mmu-miR-181b-5p | 4408.986 | −1.086 | 0.378 | −2.873 | 0.004 | 0.038 |
| mmu-miR-26a-5p | 39052.438 | −0.696 | 0.244 | −2.851 | 0.004 | 0.041 |
| mmu-miR-676-5p | 158.067 | −0.679 | 0.239 | −2.842 | 0.004 | 0.041 |
| mmu-miR-129-1-3p | 3.993 | 3.326 | 1.180 | 2.818 | 0.005 | 0.044 |
| mmu-miR-301b-3p | 100.496 | 0.727 | 0.259 | 2.808 | 0.005 | 0.044 |
| mmu-miR-1247-3p | 3.535 | 2.614 | 0.943 | 2.773 | 0.006 | 0.048 |
| mmu-miR-374b-5p | 396.776 | −1.075 | 0.388 | −2.772 | 0.006 | 0.048 |
| mmu-miR-101b-3p | 2968.919 | −0.984 | 0.358 | −2.749 | 0.006 | 0.051 |
| mmu-miR-574-5p | 57.606 | −1.152 | 0.419 | −2.748 | 0.006 | 0.051 |
| mmu-miR-1950 | 3.858 | −3.129 | 1.144 | −2.735 | 0.006 | 0.052 |
| mmu-miR-370-3p | 20.562 | 1.762 | 0.651 | 2.708 | 0.007 | 0.056 |
| mmu-miR-30e-5p | 82526.392 | −0.661 | 0.246 | −2.692 | 0.007 | 0.058 |
| mmu-miR-21a-3p | 853.156 | 0.571 | 0.214 | 2.669 | 0.008 | 0.061 |
| mmu-miR-93-5p | 44525.520 | −0.816 | 0.311 | −2.623 | 0.009 | 0.067 |
| mmu-miR-98-5p | 813.779 | −0.979 | 0.373 | −2.628 | 0.009 | 0.067 |
| mmu-miR-99a-3p | 50.722 | −0.940 | 0.358 | −2.624 | 0.009 | 0.067 |
| mmu-miR-6979-3p | 2.184 | 3.084 | 1.182 | 2.609 | 0.009 | 0.069 |
| mmu-miR-106b-5p | 3073.319 | −1.126 | 0.433 | −2.602 | 0.009 | 0.070 |
| mmu-miR-6911-3p | 9.790 | 1.381 | 0.542 | 2.548 | 0.011 | 0.081 |
| mmu-let-7d-5p | 5852.546 | −0.940 | 0.372 | −2.527 | 0.012 | 0.082 |
| mmu-miR-215-5p | 20570.662 | 0.958 | 0.379 | 2.531 | 0.011 | 0.082 |
| mmu-miR-339-5p | 903.544 | 0.809 | 0.320 | 2.525 | 0.012 | 0.082 |
| mmu-miR-465c-5p | 3.835 | 3.294 | 1.297 | 2.540 | 0.011 | 0.082 |
| mmu-miR-879-3p | 9.963 | 1.833 | 0.726 | 2.525 | 0.012 | 0.082 |
| mmu-miR-153-3p | 8.340 | 4.262 | 1.691 | 2.520 | 0.012 | 0.082 |
| mmu-miR-128-3p | 13352.037 | 0.853 | 0.345 | 2.476 | 0.013 | 0.091 |
| mmu-miR-193b-5p | 8.593 | 2.047 | 0.827 | 2.475 | 0.013 | 0.091 |
| mmu-miR-192-3p | 4.013 | 2.559 | 1.042 | 2.455 | 0.014 | 0.095 |
| mmu-miR-125a-3p | 17.818 | 1.258 | 0.515 | 2.445 | 0.014 | 0.097 |
| mmu-miR-410-3p | 353.619 | 0.883 | 0.362 | 2.440 | 0.015 | 0.097 |
| mmu-miR-195a-3p | 94.277 | −0.888 | 0.366 | −2.429 | 0.015 | 0.099 |

TABLE S6
Differentially Expressed Plasma miRNAs in CCM1 Patients vs.
Healthy non-CCM Controls (p < 0.05, non FDR-corrected)

| | baseMean | log2FoldChange | IfcSE | stat | pvalue |
|---|---|---|---|---|---|
| hsa-miR-375- | 129.647 | 1.802 | 0.613 | 2.941 | 0.003 |
| 3p | | | | | |
| hsa-let-7e- | 1415.933 | −1.327 | 0.482 | −2.755 | 0.006 |
| 5p | | | | | |
| hsa-miR-148a- | 18.913 | −2.289 | 0.835 | −2.740 | 0.006 |
| 5p | | | | | |
| hsa-miR-493- | 10.528 | 5.078 | 1.964 | 2.586 | 0.010 |
| 5p | | | | | |
| hsa-miR-19b- | 37.223 | 1.500 | 0.616 | 2.435 | 0.015 |
| 3p | | | | | |
| hsa-miR-139- | 29.769 | −1.542 | 0.680 | −2.267 | 0.023 |
| 5p | | | | | |
| hsa-miR-204- | 22.074 | −2.173 | 0.969 | −2.243 | 0.025 |
| 5p | | | | | |
| hsa-miR-4449 | 4.247 | 3.106 | 1.403 | 2.215 | 0.027 |
| hsa-miR-769- | 49.170 | −1.316 | 0.594 | −2.214 | 0.027 |
| 5p | | | | | |
| hsa-miR-4732- | 3.735 | 4.840 | 2.270 | 2.132 | 0.033 |
| 5p | | | | | |
| hsa-miR-345- | 39.746 | 1.542 | 0.739 | 2.085 | 0.037 |
| 5p | | | | | |
| hsa-miR-1301- | 38.452 | −1.257 | 0.614 | −2.047 | 0.041 |
| 3p | | | | | |
| hsa-miR-101- | 160.167 | −0.919 | 0.449 | −2.045 | 0.041 |
| 3p | | | | | |
| hsa-miR-20b- | 3.033 | 4.747 | 2.364 | 2.009 | 0.045 |
| 5p | | | | | |
| hsa-let-7b- | 4.148 | −3.661 | 1.829 | −2.002 | 0.045 |
| 3p | | | | | |
| hsa-miR-370- | 15.225 | 2.323 | 1.164 | 1.995 | 0.046 |
| 3p | | | | | |

TABLE S7
Differentially Expressed Plasma miRNAs in CCM3 Patients vs.
Healthy non-CCM Controls (p < 0.05, non FDR-corrected)

| | baseMean | log2FoldChange | IfcSE | stat | pvalue |
|---|---|---|---|---|---|
| hsa-miR- | 106.902 | 1.495 | 0.530 | 2.818 | 0.005 |
| 375-3p | | | | | |
| hsa-miR- | 44.403 | 1.912 | 0.725 | 2.636 | 0.008 |
| 4732-3p | | | | | |
| hsa-miR- | 7.447 | 3.738 | 1.486 | 2.516 | 0.012 |
| 487b-3p | | | | | |
| hsa-miR- | 20.287 | 2.053 | 0.817 | 2.513 | 0.012 |
| 625-5p | | | | | |
| hsa-miR- | 15.508 | 2.690 | 1.121 | 2.399 | 0.016 |
| 191-3p | | | | | |
| hsa-miR- | 12.493 | 3.870 | 1.623 | 2.385 | 0.017 |
| 493-5p | | | | | |
| hsa-miR- | 150.621 | −1.247 | 0.552 | −2.258 | 0.024 |
| 4433b-5p | | | | | |
| hsa-miR- | 21.784 | 2.107 | 0.938 | 2.247 | 0.025 |
| 370-3p | | | | | |
| hsa-miR- | 25.206 | −2.638 | 1.211 | −2.177 | 0.029 |
| 204-5p | | | | | |
| hsa-miR- | 5.572 | 4.261 | 1.976 | 2.157 | 0.031 |
| 369-5p | | | | | |
| hsa-miR- | 31.235 | 2.506 | 1.188 | 2.110 | 0.035 |
| 141-3p | | | | | |
| hsa-miR- | 620.537 | −0.644 | 0.321 | −2.007 | 0.045 |
| 93-5p | | | | | |

TABLE S8
Differentially Expressed Plasma miRNAs in CCM1
vs. CCM3 Patients (p < 0.05, non FDR-corrected)

| | baseMean | log2FoldChange | IfcSE | stat | pvalue |
|---|---|---|---|---|---|
| hsa-miR- | 11.699 | 3.778 | 0.989 | 3.819 | 0.000 |
| 323b-3p | | | | | |
| hsa-miR- | 1705.649 | 1.060 | 0.360 | 2.948 | 0.003 |
| 127-3p | | | | | |
| hsa-miR- | 644.962 | 1.114 | 0.377 | 2.955 | 0.003 |
| 409-3p | | | | | |
| hsa-miR- | 77.476 | 1.373 | 0.442 | 3.109 | 0.002 |
| 411-5p | | | | | |
| hsa-miR- | 235.618 | 1.293 | 0.445 | 2.906 | 0.004 |
| 432-5p | | | | | |
| hsa-miR- | 98.070 | 1.279 | 0.437 | 2.929 | 0.003 |
| 654-3p | | | | | |
| hsa-miR- | 11.326 | −3.797 | 1.267 | −2.997 | 0.003 |
| 9-5p | | | | | |
| hsa-let- | 1504.797 | 1.305 | 0.458 | 2.850 | 0.004 |
| 7e-5p | | | | | |
| hsa-miR- | 133.125 | −0.782 | 0.298 | −2.620 | 0.009 |
| 128-3p | | | | | |
| hsa-miR- | 2557.283 | 0.696 | 0.273 | 2.545 | 0.011 |
| 125a-5p | | | | | |
| hsa-miR- | 9.732 | −2.369 | 1.005 | −2.356 | 0.018 |
| 10399-3p | | | | | |
| hsa-miR- | 25.883 | −2.621 | 1.086 | −2.414 | 0.016 |
| 205-5p | | | | | |
| hsa-miR- | 335.361 | 0.937 | 0.397 | 2.359 | 0.018 |
| 379-5p | | | | | |
| hsa-miR- | 9.180 | 2.625 | 1.122 | 2.340 | 0.019 |
| 4687-5p | | | | | |
| hsa-miR- | 4.104 | 3.828 | 1.642 | 2.331 | 0.020 |
| 6770-3p | | | | | |
| hsa-miR- | 685.638 | −0.544 | 0.224 | −2.429 | 0.015 |
| 93-5p | | | | | |
| hsa-miR- | 25.736 | 2.128 | 0.965 | 2.205 | 0.027 |
| 485-3p | | | | | |
| hsa-miR- | 73.055 | 1.003 | 0.463 | 2.166 | 0.030 |
| 410-3p | | | | | |
| hsa-let- | 4.979 | 3.647 | 1.737 | 2.099 | 0.036 |
| 7b-3p | | | | | |
| hsa-miR- | 218.910 | −0.604 | 0.283 | −2.132 | 0.033 |
| 106b-3p | | | | | |
| hsa-miR- | 25.259 | 1.257 | 0.598 | 2.102 | 0.036 |
| 431-5p | | | | | |
| hsa-miR- | 38.876 | 1.475 | 0.722 | 2.042 | 0.041 |
| 342-5p | | | | | |
| hsa-miR- | 10.025 | −3.006 | 1.462 | −2.056 | 0.040 |
| 4433b-3p | | | | | |
| hsa-miR- | 13.762 | −2.080 | 1.047 | −1.986 | 0.047 |
| 1271-5p | | | | | |

TABLE S13
Differentially Expressed Plasma miRNAs in New Lesion vs.
No New Lesion Patients (p < 0.05, non FDR-corrected)

| | baseMean | log2FoldChange | IfcSE | stat | pvalue |
|---|---|---|---|---|---|
| hsa-miR- | 1377.403 | −2.018 | 0.285 | −7.093 | 0.000 |
| 342-3p | | | | | |
| hsa-let- | 1002.702 | 2.798 | 0.426 | 6.570 | 0.000 |
| 7c-5p | | | | | |
| hsa-miR- | 1437.633 | 2.545 | 0.471 | 5.404 | 0.000 |
| 30a-5p | | | | | |
| hsa-miR- | 4885.911 | −0.950 | 0.187 | −5.085 | 0.000 |
| 126-5p | | | | | |
| hsa-miR- | 353.790 | −2.220 | 0.437 | −5.083 | 0.000 |
| 363-3p | | | | | |
| hsa-miR- | 37.267 | −3.511 | 0.691 | −5.082 | 0.000 |
| 150-3p | | | | | |
| hsa-miR- | 93.987 | 2.935 | 0.619 | 4.738 | 0.000 |
| 133a-3p | | | | | |
| hsa-miR- | 2399.381 | 2.198 | 0.464 | 4.735 | 0.000 |
| 192-5p | | | | | |
| cel-miR- | 15.653 | −4.859 | 1.057 | −4.599 | 0.000 |
| 39-3p | | | | | |
| hsa-miR- | 39.579 | 2.729 | 0.632 | 4.317 | 0.000 |
| 194-5p | | | | | |
| hsa-let- | 2641.085 | 0.735 | 0.174 | 4.225 | 0.000 |
| 7i-5p | | | | | |
| hsa-miR- | 50.381 | −1.514 | 0.362 | −4.180 | 0.000 |
| 130b-5p | | | | | |
| hsa-miR- | 425.225 | −1.214 | 0.293 | −4.146 | 0.000 |
| 425-5p | | | | | |
| hsa-miR- | 4.655 | 6.195 | 1.510 | 4.104 | 0.000 |
| 424-5p | | | | | |
| hsa-miR- | 30.212 | 5.919 | 1.445 | 4.095 | 0.000 |
| 205-5p | | | | | |
| hsa-miR- | 793.260 | −1.104 | 0.272 | −4.056 | 0.000 |
| 941 | | | | | |
| hsa-miR- | 6.658 | −5.611 | 1.390 | −4.036 | 0.000 |
| 668-3p | | | | | |
| hsa-miR- | 3888.231 | 1.840 | 0.462 | 3.981 | 0.000 |
| 98-5p | | | | | |
| hsa-miR- | 104.322 | 2.431 | 0.648 | 3.753 | 0.000 |
| 29a-3p | | | | | |
| hsa-miR- | 1129.257 | 0.991 | 0.264 | 3.748 | 0.000 |
| 744-5p | | | | | |
| hsa-miR- | 1112.544 | −1.670 | 0.457 | −3.650 | 0.000 |
| 150-5p | | | | | |
| hsa-miR- | 38.197 | −1.897 | 0.535 | −3.547 | 0.000 |
| 342-5p | | | | | |
| hsa-miR- | 10.987 | 3.706 | 1.052 | 3.524 | 0.000 |
| 1271-5p | | | | | |
| hsa-miR- | 33.461 | −2.750 | 0.787 | −3.496 | 0.000 |
| 485-3p | | | | | |
| hsa-let- | 57302.936 | 1.387 | 0.403 | 3.442 | 0.001 |
| 7a-5p | | | | | |
| cel-let- | 57229.298 | 1.387 | 0.403 | 3.441 | 0.001 |
| 7-5p | | | | | |
| hsa-miR- | 13.719 | 3.006 | 0.882 | 3.408 | 0.001 |
| 24-2-5p | | | | | |
| hsa-miR- | 299.797 | −0.665 | 0.196 | −3.393 | 0.001 |
| 223-3p | | | | | |
| hsa-miR- | 2247.092 | 0.722 | 0.214 | 3.369 | 0.001 |
| 27b-3p | | | | | |
| hsa-miR- | 7.643 | −3.500 | 1.044 | −3.353 | 0.001 |
| 873-3p | | | | | |
| hsa-miR- | 336698.261 | −0.730 | 0.220 | −3.320 | 0.001 |
| 191-5p | | | | | |
| hsa-miR- | 1161.600 | 0.836 | 0.257 | 3.251 | 0.001 |
| 148a-3p | | | | | |
| hsa-miR- | 7.433 | −2.861 | 0.882 | −3.244 | 0.001 |
| 10399-3p | | | | | |
| hsa-miR- | 4.510 | −4.571 | 1.419 | −3.220 | 0.001 |
| 1185-1-3p | | | | | |
| hsa-miR- | 3.795 | 4.840 | 1.503 | 3.220 | 0.001 |
| 34c-5p | | | | | |
| hsa-miR- | 46.887 | 1.412 | 0.454 | 3.106 | 0.002 |
| 340-5p | | | | | |
| hsa-miR- | 14.940 | 2.265 | 0.730 | 3.102 | 0.002 |
| 2110 | | | | | |
| hsa-miR- | 60.418 | −1.500 | 0.488 | −3.072 | 0.002 |
| 345-5p | | | | | |
| hsa-miR- | 1757.533 | 1.112 | 0.364 | 3.059 | 0.002 |
| 143-3p | | | | | |
| hsa-miR- | 6.780 | −3.683 | 1.206 | −3.053 | 0.002 |
| 1180-3p | | | | | |
| hsa-miR- | 2731211.948 | 1.343 | 0.440 | 3.052 | 0.002 |
| 486-5p | | | | | |
| hsa-miR- | 7.235 | 3.838 | 1.270 | 3.023 | 0.003 |
| 31-5p | | | | | |
| hsa-miR- | 31.614 | −2.219 | 0.742 | −2.993 | 0.003 |
| 424-3p | | | | | |
| hsa-let- | 1413.186 | 0.863 | 0.292 | 2.957 | 0.003 |
| 7g-5p | | | | | |
| hsa-miR- | 121.065 | −1.375 | 0.467 | −2.945 | 0.003 |
| 654-3p | | | | | |
| hsa-miR- | 151.769 | 1.012 | 0.345 | 2.929 | 0.003 |
| 128-3p | | | | | |
| hsa-miR- | 21.928 | 1.711 | 0.584 | 2.928 | 0.003 |
| 654-5p | | | | | |
| hsa-miR- | 137.127 | −0.989 | 0.341 | −2.904 | 0.004 |
| 3615 | | | | | |
| hsa-miR- | 355.104 | 1.127 | 0.389 | 2.900 | 0.004 |
| 378a-3p | | | | | |
| hsa-miR- | 266.866 | 1.041 | 0.362 | 2.877 | 0.004 |
| 501-3p | | | | | |
| hsa-miR- | 3.036 | −4.479 | 1.560 | −2.872 | 0.004 |
| 576-5p | | | | | |
| hsa-miR- | 1754.936 | 0.688 | 0.241 | 2.860 | 0.004 |
| 30e-5p | | | | | |
| hsa-miR- | 459.093 | 1.162 | 0.418 | 2.780 | 0.005 |
| 379-5p | | | | | |
| hsa-miR- | 3.444 | −4.664 | 1.687 | −2.764 | 0.006 |
| 323a-3p | | | | | |
| hsa-miR- | 120.230 | −1.052 | 0.380 | −2.764 | 0.006 |
| 7706 | | | | | |
| hsa-miR- | 1865.588 | 0.748 | 0.275 | 2.716 | 0.007 |
| 21-5p | | | | | |
| hsa-miR- | 35.030 | −1.336 | 0.496 | −2.695 | 0.007 |
| 625-3p | | | | | |
| hsa-miR- | 72.906 | −1.043 | 0.391 | −2.671 | 0.008 |
| 28-5p | | | | | |
| hsa-let- | 9805.586 | 0.835 | 0.316 | 2.638 | 0.008 |
| 7f-5p | | | | | |
| hsa-miR- | 1013.099 | 0.632 | 0.241 | 2.619 | 0.009 |
| 222-3p | | | | | |
| hsa-miR- | 93.677 | 0.904 | 0.350 | 2.585 | 0.010 |
| 139-3p | | | | | |
| hsa-miR- | 551.408 | 0.745 | 0.289 | 2.574 | 0.010 |
| 140-3p | | | | | |
| hsa-miR- | 806.092 | −0.951 | 0.370 | −2.569 | 0.010 |
| 409-3p | | | | | |
| hsa-miR- | 9.675 | −2.216 | 0.872 | −2.541 | 0.011 |
| 550a-5p | | | | | |
| hsa-miR- | 11.256 | 3.332 | 1.315 | 2.534 | 0.011 |
| 9-5p | | | | | |
| hsa-miR- | 2.015 | −3.895 | 1.548 | −2.517 | 0.012 |
| 23c | | | | | |
| hsa-miR- | 269.977 | −0.793 | 0.321 | −2.473 | 0.013 |
| 197-3p | | | | | |
| hsa-miR- | 4.381 | 3.945 | 1.603 | 2.461 | 0.014 |
| 6770-3p | | | | | |
| hsa-miR- | 3.702 | −3.997 | 1.628 | −2.455 | 0.014 |
| 32-5p | | | | | |
| hsa-miR- | 34353.969 | −0.882 | 0.362 | −2.437 | 0.015 |
| 10b-5p | | | | | |
| hsa-miR- | 29.050 | −1.488 | 0.612 | −2.430 | 0.015 |
| 146b-3p | | | | | |
| hsa-miR- | 7.596 | −3.035 | 1.261 | −2.407 | 0.016 |
| 369-5p | | | | | |
| hsa-miR- | 1327.170 | 0.537 | 0.231 | 2.330 | 0.020 |
| 103a-3p | | | | | |
| hsa-miR- | 165.559 | 0.779 | 0.336 | 2.317 | 0.021 |
| 4433b-5p | | | | | |
| hsa-miR- | 27.546 | 1.648 | 0.716 | 2.302 | 0.021 |
| 141-3p | | | | | |
| hsa-miR- | 136.924 | 1.004 | 0.437 | 2.299 | 0.022 |
| 181c-5p | | | | | |
| hsa-miR- | 1328.366 | −0.630 | 0.280 | −2.252 | 0.024 |
| 484 | | | | | |
| hsa-miR- | 3.597 | −3.150 | 1.413 | −2.230 | 0.026 |
| 12136 | | | | | |
| hsa-miR- | 39.005 | 0.984 | 0.443 | 2.220 | 0.026 |
| 20a-5p | | | | | |
| hsa-miR- | 4.256 | −3.190 | 1.446 | −2.206 | 0.027 |
| 577 | | | | | |
| hsa-miR- | 61.355 | 1.201 | 0.546 | 2.199 | 0.028 |
| 125b-5p | | | | | |
| hsa-miR- | 4.486 | −2.988 | 1.362 | −2.194 | 0.028 |
| 1343-3p | | | | | |
| hsa-miR- | 451.670 | −1.017 | 0.466 | −2.184 | 0.029 |
| 183-5p | | | | | |
| hsa-miR- | 29.119 | −1.308 | 0.604 | −2.166 | 0.030 |
| 431-5p | | | | | |
| hsa-miR- | 29135.999 | −0.522 | 0.241 | −2.165 | 0.030 |
| 10a-5p | | | | | |
| hsa-miR- | 4.585 | −3.292 | 1.534 | −2.146 | 0.032 |
| 125b-2-3p | | | | | |
| hsa-miR- | 6.396 | 2.629 | 1.245 | 2.111 | 0.035 |
| 195-5p | | | | | |
| hsa-miR- | 11.109 | 1.547 | 0.733 | 2.110 | 0.035 |
| 23a-5p | | | | | |
| hsa-miR- | 1386.242 | −0.552 | 0.264 | −2.087 | 0.037 |
| 92b-3p | | | | | |
| hsa-miR- | 7.625 | −2.964 | 1.440 | −2.059 | 0.039 |
| 4326 | | | | | |
| hsa-miR- | 6.637 | −2.049 | 0.998 | −2.054 | 0.040 |
| 543 | | | | | |
| hsa-miR- | 21.842 | −1.790 | 0.882 | −2.030 | 0.042 |
| 550a-3p | | | | | |
| hsa-miR- | 4.026 | −3.331 | 1.654 | −2.015 | 0.044 |
| 3677-3p | | | | | |
| hsa-let- | 3.039 | −3.683 | 1.840 | −2.001 | 0.045 |
| 7e-3p | | | | | |
| hsa-miR- | 7.992 | −2.322 | 1.166 | −1.991 | 0.046 |
| 5010-3p | | | | | |
| hsa-miR- | 88.525 | −0.822 | 0.415 | −1.981 | 0.048 |
| 100-5p | | | | | |
| hsa-miR- | 6.162 | 2.909 | 1.473 | 1.974 | 0.048 |
| 125b-1-3p | | | | | |

### XI. Kits

The present invention also concern kits containing compositions of the invention or compositions to implement methods of the invention. Kits can be used to evaluate one or more biomarkers. A kit may contain, contains at least or contains at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 100, 500, 1,000 or more probes, primers or primer sets, synthetic molecules, antibodies, or inhibitors, or any value or range and combination derivable therein. Also provided are kits for evaluating biomarker activity or level in a cell.

Kits may comprise components, which may be individually packaged or placed in a container, such as a tube, bottle, vial, syringe, or other suitable container means.

Individual components may also be provided in a kit in concentrated amounts; a component may be provided individually in the same concentration as it would be in a solution with other components. Concentrations of components may be provided as 1×, 2×, 5×, 10×, or 20× or more.

Kits for using probes, antibodies, synthetic nucleic acids, nonsynthetic nucleic acids, and/or inhibitors of the disclosure for prognostic or diagnostic applications are included as part of the disclosure. Specifically contemplated are any such molecules corresponding to any biomarker identified herein, which includes antibodies that bind to such biomarkers as well as nucleic acid primers/primer sets and probes that are identical to or complementary to all or part of a biomarker, which may include noncoding sequences of the biomarker, as well as coding sequences of the biomarker.

Negative and/or positive control nucleic acids, antibodies, probes, and inhibitors may be included. In addition, a kit may include a sample that is a negative or positive control for methylation of one or more biomarkers.

It is contemplated that any method or composition described herein can be implemented with respect to any other method or composition described herein and that different embodiments may be combined. The claims originally filed are contemplated to cover claims that are multiply dependent on any filed claim or combination of filed claims.

### XII. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1: Circulating Plasma miRNA Homologs in Mice and Humans Reflect Familial Cerebral Cavernous Malformation Disease

Patients with familial cerebral cavernous malformation (CCM) inherit germline loss of function mutations and are susceptible to progressive development of brain lesions and neurological sequelae during their lifetime. To date, no homologous circulating molecules have been identified that can reflect the presence of a CCM mutation, both in animal models and patients. The inventors hypothesize that homologous differentially expressed (DE) plasma miRNAs can reflect the CCM germline mutation in preclinical murine models and patients and can be applied to develop translatable circulating biomarkers. Herein, several homologous DE plasma miRNAs with mechanistic putative gene targets within the transcriptome of preclinical and human CCM lesions were identified. Several of these gene targets were additionally found to be associated with CCM-enriched pathways identified using the Kyoto Encyclopedia of Genes and Genomes. DE miRNAs were also identified in familial CCM patients who developed new brain lesions within the year following blood sample collection. The miRNome results were then validated in an independent cohort of human subjects with real-time-qPCR quantification, a technique facilitating plasma assays. Finally, a Bayesian-informed machine learning approach showed that a combination of plasma levels of miRNAs and circulating proteins improves their association with familial-CCM disease in human subjects to 95% accuracy. These findings act as an important proof of concept for developing translatable circulating biomarkers of CCM disease in mice and humans. These can be applied in preclinical studies and human trials aimed at monitoring and restoring gene function in CCM and other diseases.

Cerebral cavernous malformations (CCMs), also known as cavernous angiomas, are enlarged blood-filled vascular caverns prone to hemorrhage due to dysfunctional vessel wall angioarchitecture affecting up to 0.5% of the population [1, 2]. Approximately 30% of CCM patients manifest familial disease, with an autosomal dominant inheritance of a heterozygous germline loss of function mutation in one of three genes, CCM1/KRIT1, CCM2/Malcavernin, or CCM3/PDCD10 [1]. These familial cases develop new hemorrhagic brain lesions throughout their life, predisposing them to a risk of symptomatic hemorrhage, seizures, and/or focal neurological deficits [1]. The CCM3/PDCD10 disease manifests exceptional aggressiveness with greater lesion burden and earlier disease manifestations than other genotypes [3]. Surgical excision of symptomatic CCM lesions is the only current therapeutic option, with serious morbidity and costs, and obvious limitations with multiple lesions [1]. Thus, there is an intense effort at developing novel therapies [4, 5], including gene restoration therapies with viral vectors for familial disease [6].

There is an ongoing search for facile biomarkers that can accurately reflect disease status and lesional activity of CCMs, to guide the selection of aggressive cases for clinical trials and to monitor the impact of novel therapies [7-10]. Peripheral blood plasma provides a unique window into greater systemic biological processes, offering a promising opportunity for biomarker development [7, 11, 12]. In fact, dysregulated lesional pathways identified in CCM lesions may be reflected within the plasma [13]. Recent studies in preclinical murine models and human CCM patients have reported a differential plasma proteome in CCM disease, including proteins which have previously been implicated in CCM mechanisms [14-20], and reflect the transcriptome of micro-dissected human and murine CCM lesions [13, 21]. However, biomarker discovery in CCM has not systematically examined circulating molecules other than plasma proteins.

Overall, it is beneficial to identify circulating homologous miRNAs with postulated mechanistic links, which can efficiently be cross validated in preclinical models of disease and translated to human subjects [27]. This is especially advantageous where experimental therapeutics could be tested, and biomarker efficacy monitored using the same biomarker in preclinical studies, in human trials and ultimately in clinical practice. To date, homologous plasma miRNAs which reflect disease states in murine models and humans have not yet been identified. The inventors hypothesize that homologous DE plasma miRNAs can reflect the heterozygous inherited CCM germline mutation in preclinical murine models and patients, can distinguish cases who would develop new lesions, and can be assayed in peripheral blood and thus developed as translatable circulating biomarkers in specific clinical contexts of use.

### A. Methods

### 1. Mouse Sample Collection

Blood was collected via the submandibular vein route from 3 to 5 month-old mice including 7 Ccm1^{+/-} mice, 7 Ccm3^{+/-} mice and 7 sex matched wild type mice. These mice were bred at the University of Chicago (Table A). The pre-clinical heterozygous mice have been used to more closely mimic the physiopathology of the human familial disease [3].

TABLE A
Summary of Mouse Experimental Information
used for the Plasma miRNome Sequencing

| | | Mice bred | Mice bred |
|---|---|---|---|
| | | University of | University of |
| | | Chicago | California San Diego |
| Experimental | Background | C57BL/6 | C57BL/6 |
| Mice | Genotype | Ccm1^{+/-} | Ccm1^{fl/fl} with Pdgfb- |
| | | (n = 7) | Cre⁺ (n = 7) |
| | | Ccm3^{+/-} | Ccm3^{fl/fl} with Pdgfb- |
| | | (n = 7) | Cre⁺ (n = 5) |
| | Induction | N/A | 50 μg 4-hydroxy-tamoxifen |
| | | | P1 for Ccm1^{-/-}, |
| | | | P6 for Ccm3^{+/-} |
| | Age | 3 to 5 months | 7-10 weeks |
| Control Mice | Background | C57BL/6 | C57BL/6 |
| | Genotype | Ccm1^{+/+} and | Ccm1^{fl/fl} with Pdgfb-Cre⁻ |
| | | Ccm3^{+/+} | Ccm3^{fl/fl} with Pdgfb-Cre⁻ |
| | | (n = 7) | (n = 8) |
| | Induction | N/A | 50 μg 4-hydroxy-tamoxifen |
| | | | at Pl (n = 5) or P6 (n = 3) |
| | Age | 3 to 5 months | 7-10 weeks |

Seven Ccm1^{flox/flox} mice and 5 Ccm3^{flox/flox} mice with a Pdgfb-Cre⁺ driver, along with 8 sex matched wild type mice were bred at the University of California San Diego (Table A). These mice were injected with 50 μg of 4-hydroxy-tamoxifen intraperitoneally at P1 to induce Cre activity and gene loss, bypassing embryonic lethality of the homozygous state [28]. These mice and their controls were sacrificed uniformly at 7-10 weeks of age through cardiac puncture and blood was collected simultaneously.

After collection, plasma was separated using Z-gel tubes (Sarstedt, Nümbrecht, Germany) through centrifugation (AllegraX-30R, Beckman Coulter). One hundred ul of plasma was then aliquoted into 1.7-ml microcentrifuge tubes and stored at −80° C. until RNA extraction. The sample size for this study was estimated based on a previous study that identified DE miRNAs in the plasma of only 3 Ccm3^{+/-} mice when compared to 3 wild type mice [21].

### 2. Human CCM Cohort Characteristics

For this study, the plasma miRNome of 23 familial-CCM patients (13 CCM1 and 10 CCM3), enrolled between July 2014 and July 2019, was sequenced (Table 1). The diagnosis of CCM was established via magnetic resonance imaging (MRI) at a single referral center (uchicagomedicine.org/ccm) by a senior neurosurgeon (IAA) with more than 30 years of experience in CCM disease management. The CCM genotype was defined as either CCM1 or CCM3, through genetic testing performed by PreventionGenetics (Marshfield, WI, USA), utilizing Sanger and NextGen sequencing followed by deletion/duplication analysis [2]. Patients with partial or complete resection of CCM or any prior brain irradiation were excluded. Due to low numbers of CCM2 cases, which only make up 20% of familial cases, this genotype was unable to be analyzed separately [29]. Among the 23 familial-CCM patients enrolled, 10 (5 CCM1 and 5 CCM3) harbored at least 1 new CCM on their routine clinical follow-up SWI MRI in the year following their blood sample collection. SWI imaging is recommended for the diagnosis of CCM and assessment of lesion burden as this sequence is more sensitive in detecting perilesional hemosiderin and capturing smaller lesions than traditional T1 and T2 MRI sequences [2, 30].

TABLE 1
Cohort Demographics of CCM Patients and
Healthy Controls for miRNome Sequencing.

| | | | Healthy |
|---|---|---|---|
| Patient Characteristics | CCM1 | CCM3 | non-CCM |
| Sample Size | 13 | 10 | 7 |
| Age (mean ± SD) | 30.9 ± 21.9 | 24.1 ± 13.5 | 22.1 ± 4.1 |
| Range (yr.) | [4.3-59.5] | [10.2-62.3] | [19.1-30.6] |
| Female (%) | 53.8 | 60.0 | 57.1 |
| Lesion Characteristics | | | |
| Number of SWI-weighted | 32.2 ± 39.4 | 44.0 ± 26.5 | NA |
| lesions (mean ± SD) | | | |
| Range | [4-105] | [10-101] | |
| Number of T₂-weighted | 7.1 ± 7.4 | 18.9 ± 11.0 | NA |
| lesions (mean ± SD) | | | |
| Range | [2-23] | [5-39] | |
| Patients with T₂ | 23.0 | 10.0 | NA |
| brainstem lesions (%) | | | |
| Ethnicity | | | |
| White/European (%) | 53.8 | 100 | 28.6 |
| African American (%) | 23.1 | 0 | 14.2 |
| Hispanic (%) | 23.1 | 0 | 28.6 |
| Asian (%) | 0 | 0 | 28.6 |

SWI = susceptibility weighted imaging;
SD = standard deviation,
NA = not applicable

An independent cohort including 24 familial-CCM patients (12 CCM1 and 12 CCM3) and 7 healthy non-CCM subjects was also enrolled between July 2014 and July 2019 for validation of the miRNA sequencing using real time quantitative PCR (RT-qPCR) (Table 2). Demographic analyses showed that a greater proportion of white/Caucasian was enrolled in both cohorts of CCM patients [p<0.05]. No other statistically significant demographic difference was observed between any of the human CCM cohorts, including age and sex. Previous statistical simulations and clinical studies have reported several DE plasma miRNAs in CCM patients using similar sample sizes [7, 26].

TABLE 2
Cohort Demographics of CCM Patients and
Healthy Controls for RT-qPCR Validation.

| | | | Healthy |
|---|---|---|---|
| Patient Characteristics | CCM1 | CCM3 | non-CCM |
| Sample Size | 12 | 12 | 13 |
| Age (mean ± SD) | 43.7 ± 20.8 | 28.5 ± 19.0 | 39.7 ± 14.7 |
| Range (yr.) | [12.2-67.5] | [7.0-64.7] | [19.3-63.6] |
| Female (%) | 83.3 | 41.7 | 53.8 |
| Lesion Characteristics | | | |
| Number of SWI-weighted | 49.3 ± 46.3 | 36.2 ± 33.0 | NA |
| lesions (mean ± SD) | | | |
| Range | [1-106] | [9-101] | |
| Number of T₂-weighted | 6.9 ± 7.6 | 11.0 ± 6.6 | NA |
| lesions (mean ± SD) | | | |
| Range | [0-23] | [3-22] | |
| Patients with T₂ | 33.3 | 8.3 | NA |
| brainstem lesions (%) | | | |
| Ethnicity | | | |
| White/European (%) | 91.7 | 91.7 | 53.5 |
| African American (%) | 8.3 | 0 | 23.0 |
| Hispanic (%) | 0 | 0 | 8.2 |
| Asian (%) | 0 | 8.3 | 15.3 |

SWI = susceptibility weighted imaging;
SD = standard deviation,
NA = not applicable

Healthy non-CCM subjects were excluded if they had (a) any medical or neurologic condition requiring ongoing follow-up or medical treatment in the preceding year, (b) a history of concussion or brain trauma in the preceding year, (c) a history of prior brain irradiation at any time, (d) been pregnant or lactating in the preceding year, (e) used recreational, psychoactive, or neuroleptic drugs in the prior year. The healthy non-CCM subjects had an MRI performed to ensure that they did not have any unknown neurological conditions that could confound the study.

3. miRNA Sequencing and Differential Expression Analyses

Total RNAs from the serum of both heterozygous and homozygous mice (200 ul), and from the plasma of familial-CCM patients (up to 100 ul) were extracted using the miRNeasy Serum/Plasma Kit (Qiagen, Hilden, Germany) following the manufacturer recommendation [21, 26]. cDNA libraries were then generated with commercially available Illumina small RNA-Seq kits (Clontech, Mountain View, CA, USA) and sequenced with the Illumina HiSeq 4000 platform (Illumina, San Diego, CA, USA) using single-end 50-bp reads, at the University of Chicago Genomics Core for sequencing. The sequencing depth median for the plasma miRNomes was 10 million and 12 million reads per sample for mouse and human analyses, respectively. The sequencing depth used herein remains within reasonable range to identify known miRNAs that are differentially expressed between groups [31].

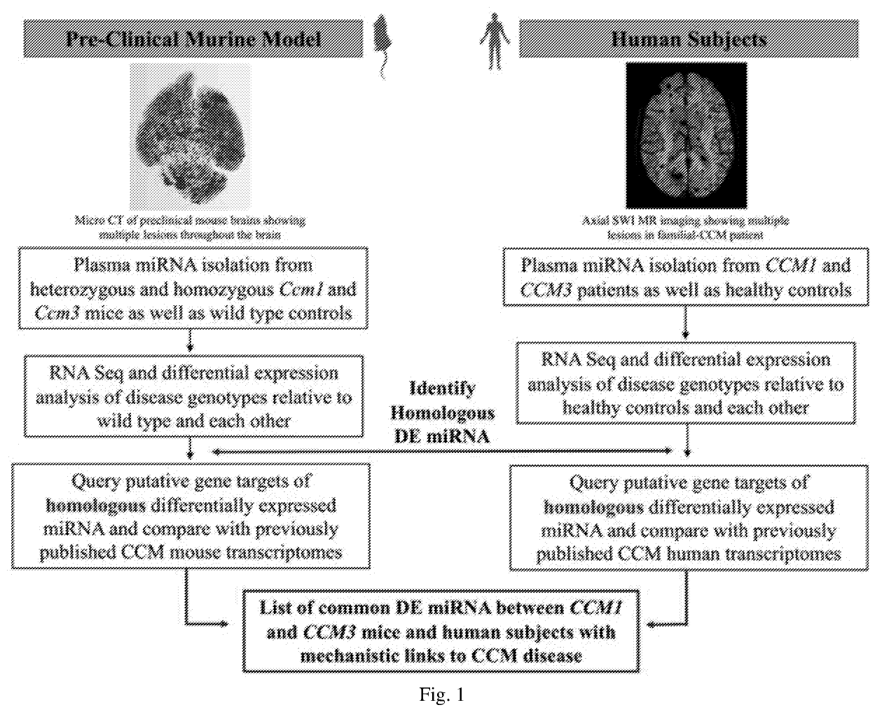

The differential expression analyses were first performed in the plasma of the preclinical mouse models between (1) Ccm1^{-/-}, (2) Ccm1^{+/-}, (3) Ccm3^{-/-}, (4) Ccm3^{+/-} and their respective wild type controls [p<0.1, false discovery rate (FDR) corrected]. Additional differential expression analyses were done between (1) Ccm1′ and Ccm3-4, and (2) Ccm1+/− and Ccm3+/(FIG. 1).

Differential expression analyses were performed in human between healthy non-CCM controls and (1) CCM1 patients, (2) CCM3 patients, and then (3) between CCM1 and CCM3 patients (FIG. 1). An additional analysis was performed between the CCM patients showing New Lesion Formation and No New Lesion Formation. Though these results were not FDR corrected, additional stringency was added as only homologous DE miRNA with (1) gene targets within CCM transcriptomes and (2) mechanistic links reported in CCM disease were considered most relevant. All differential expression analyses were conducted using R bioconductor package DESeq2. Additionally, a partial least-squares discriminant analysis (PLS-DA) was used to validate the capacity of DE miRNAs (p<0.05, non-FDR corrected) to distinguish between CCM1, CCM3, and healthy non-CCM [32-34]. Using the mixOmics package in R [35], PLS-DA components were sorted based on the variance explained, and the AUC and p-value were reported for the top 2 components of each genotype comparison.

4. Putative Targets of Homologous Mouse-Human DE miRNAs

The human homologs of DE miRNAs identified in preclinical murine models were queried using the MirGeneDB database (found online at mirgenedb.org/) (FIG. 1). Putative gene targets of homologous mouse-human DE miRNAs were identified using miRWalk 3.0 [36, 37] (FIG. 1). For each miRNA, gene targets were identified for the 3 different gene locations (3′ untranslated region (UTR), 5′ UTR, and coding sequence (CDS)) using a random forest tree algorithm with a bonding prediction probability higher than 95%. All known gene sequences are scanned and any matches to known miRNA sequences are flagged. These matches are then compared and validated using 8 other established miRNA-target prediction programs [38]. MiR Walk also consolidates data from several databases, including TargetScan, miRDB, and miRTarBase, and reports known miRNA-gene interactions from the literature to provide both predicted and validated gene target binding sites of miRNAs [38]. Only genes that appeared in at least 2 of the 3 databases on miRWalk were considered to be putative gene targets of the homologous DE miRNA, ensuring these interactions were validated by more than one source [13, 21, 26].

The putative targets of each DE miRNA identified in the plasma of mouse Ccm1^{-/-} and Ccm1^{+/-} were queried within the transcriptome of in vitro Ccm1^{-/-} mouse brain microvascular endothelial cells (BMECs). While the putative targets of the DE miRNAs identified in the plasma of Ccm3^{-/-} and Ccm3^{+/-} mice were queried in the transcriptome of (1) in vitro Ccm3^{-/-} mouse BMECs, and (2) laser micro-dissected lesional neurovascular units (NVUs) of Ccm3^{+/-} mice. Gene targets of DE miRNAs identified in the plasma of CCM1 and CCM3 patients were queried within the transcriptome of laser micro-dissected NVUs of human surgically resected CCM lesions.

### 5. CCM-Enriched Kyoto Encyclopedia of Genes and Genomes (KEGG) Pathway Analyses

The enriched KEGG pathways of each transcriptome were derived independently using the differentially expressed genes (DEGs) [p<0.05, FDR corrected; |FC|>1.5] identified in (1) Ccm14-BMECs, (2) Ccm3-BMECs, as well as micro-dissected lesional NVUs of (3) Ccm3^{+/-} mice, and (4) human surgically resected CCMs. Enriched KEGG pathways [p<0.05, FDR corrected] were generated using a database and knowledge extraction engine with a Bayes factor cutoff of 3. The putative target DEGs of each homologous DE miRNA were then associated with the list of CCM-enriched KEGG pathways within their respective transcriptomes. Finally, KEGG pathways and putative target DEGs were categorized into 6 biological processes related to CCM disease identified after a comprehensive literature search [21]. These biological processes include: (1) Neuron, Glia, Pericyte Function, (2) Permeability/Adhesion, (3) Apoptosis and Oxidative Stress, (4) Inflammation/Immune Response, (5) Vascular Processes, (6) Cellular Proliferation.

6. Selection of miRNAs for Validation Using RT-qPCR

RT-qPCR was not only used to validate the miRNA sequencing results but also serve as proof of feasibility for direct measurement of miRNAs in plasma for further biomarker use. Validation of potential biomarker candidates is needed for eventual biomarker development [40]. Among the homologous DE plasma miRNA with gene targets within CCM transcriptomes, 4 were chosen for RT-qPCR validation as (1) they have at least 10 DEGs [p<0.05, FDR corrected] as putative targets within the human CCM lesional transcriptome, and (2) had putative gene targets previously implicated in CCM disease. In addition, miR-375-5p was assessed because it was dysregulated across both CCM1 and CCM3 genotypes in the plasma of both preclinical murine models and CCM patients. Finally, not DE in mice, let-7e-5p was selected since it was the only miRNA dysregulated in CCM1 patients when compared to healthy controls as well as CCM3 patients, making it a potential specific marker for the CCM1 genotype.

7. RT-qPCR Assay of Putative DE miRNAs

Relative and absolute quantifications of a panel of 6 pre-selected DE miRNAs were assessed using RT-qPCR. The total RNA was first extracted from plasma using the MagMAX mirVana Total RNA Isolation Kit (Thermo Fisher, Waltman, MA, USA) An exogenous spike-in control, miR-cel-39-3p (1.5×10¹⁰ copies of cel-miR-39-3p in 5 μl) (IDT, Coralville, IA, USA), was added to all plasma samples prior to extraction and then measured to correct for extraction efficiency, as well as to ensure the inter-plate reproducibility of the RT-qPCR [41, 42]. RT-qPCR was performed using the TaqMan Advanced miRNA Assays Kit (Thermo Fisher). A miR-Amp reaction was performed to amplify miRNAs prior to real-time PCR using the QuantStudio3 (Thermo Fisher). The absolute quantification (number of miRNAs strands/μl) of each miRNA was estimated using a standard curve comprised of serial dilutions of 6 known concentrations of the miRNAs of interest (10⁵ to 10¹⁰ copies/μl).

For the relative quantification, the Cq values of each selected plasma miRNA as well as of two human endogenous controls, miR423-5p and miR16-5p, and of exogenous control cel-miR39-3p were measured. MiR-423-5p was eventually used as the endogenous control as it is expected to be expressed at equal levels across tissue types and samples [43]. This was validated using the NormFinder software, as miR-423-5p was shown to have the most stable expression across the controls tested (found online at moma.dk/normfinder-software) [44]. The Cq values of the endogenous control were then used to calculate the relative plasma expression [45]:

$$Δ Cq = {Cq}_{miRNA of interest} - {Cq}_{endogenous control}$$

The comparison of the relative quantification of each pre-defined miRNA was performed between (1) familial-CCM1 patients, (2) familial-CCM3 patients, and (3) healthy non-CCM controls. For each miRNA, a relative quantification value greater than +3 standard deviations away from the mean was defined as an outlier [46, 47]. A non-parametric Mann-Whitney was performed between the ΔCq for each comparison to estimate the ΔΔCq using Prism (GraphPad, San Diego, California) [48].

8. Combined Plasma miRNA and Protein Biomarker Development

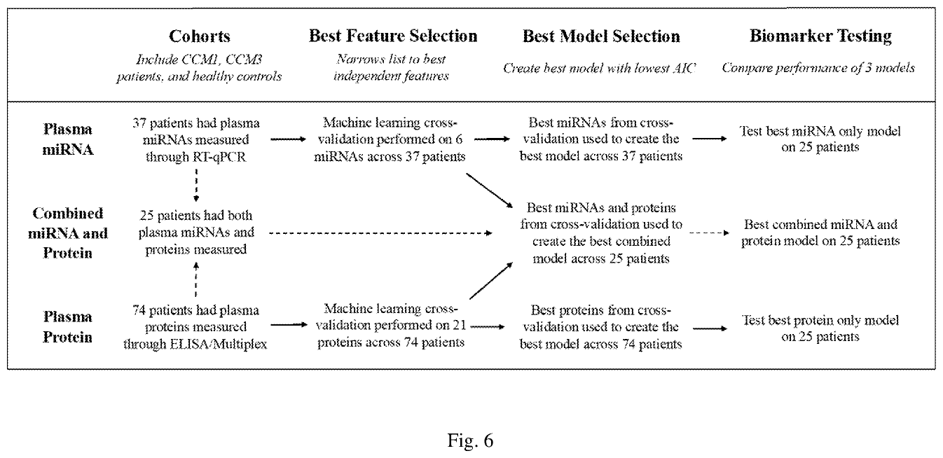

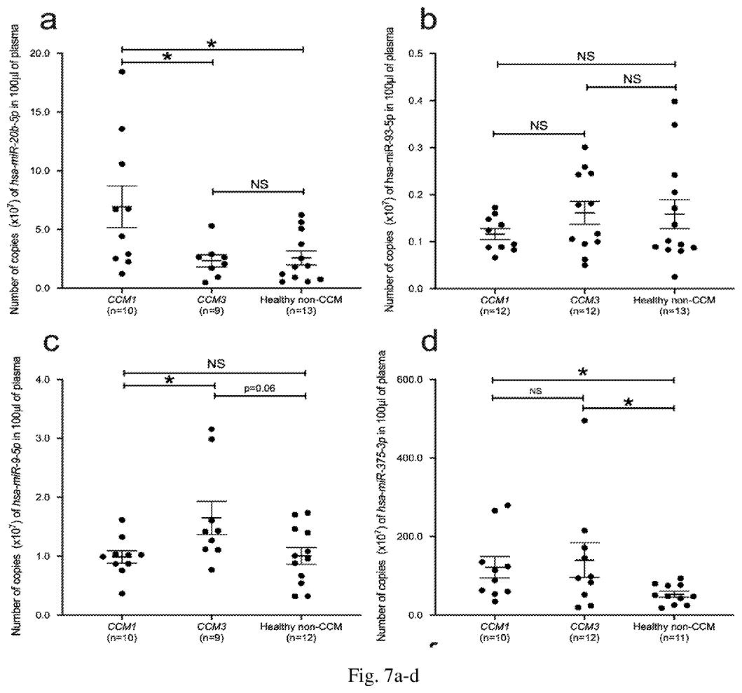

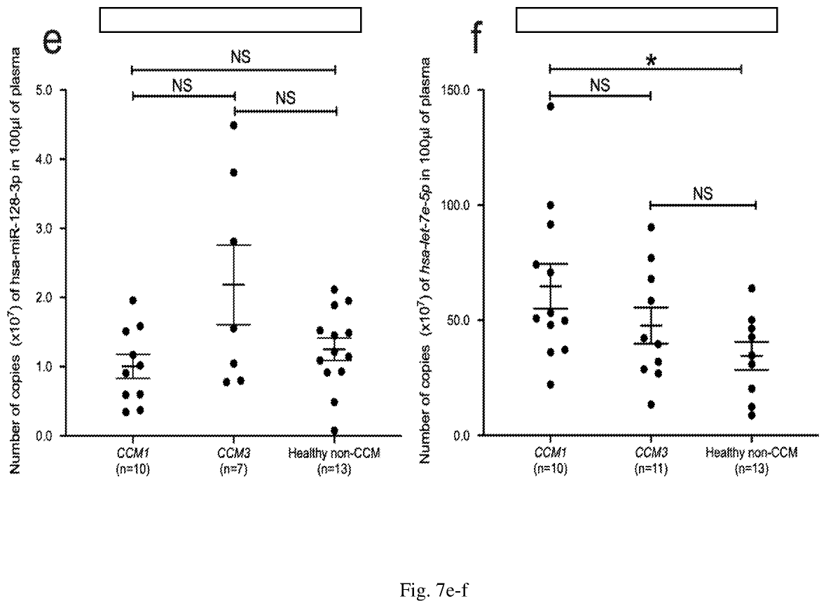

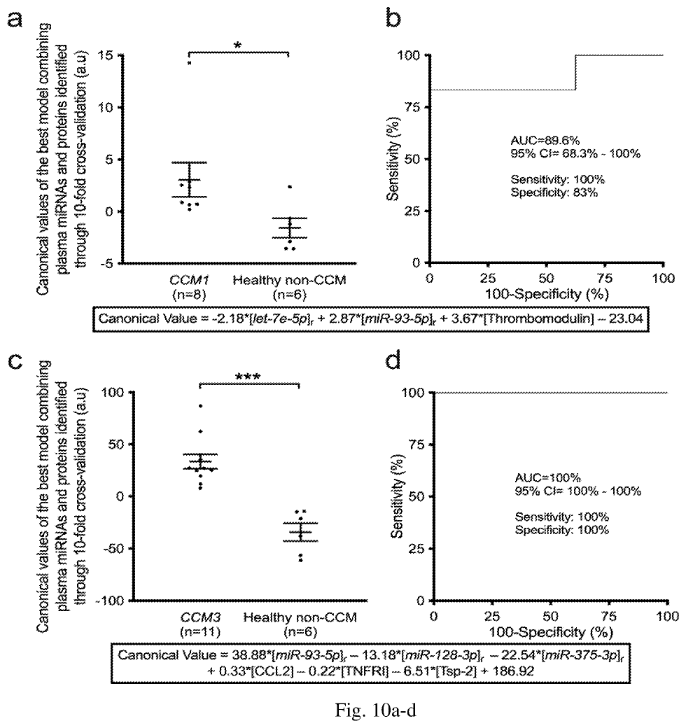

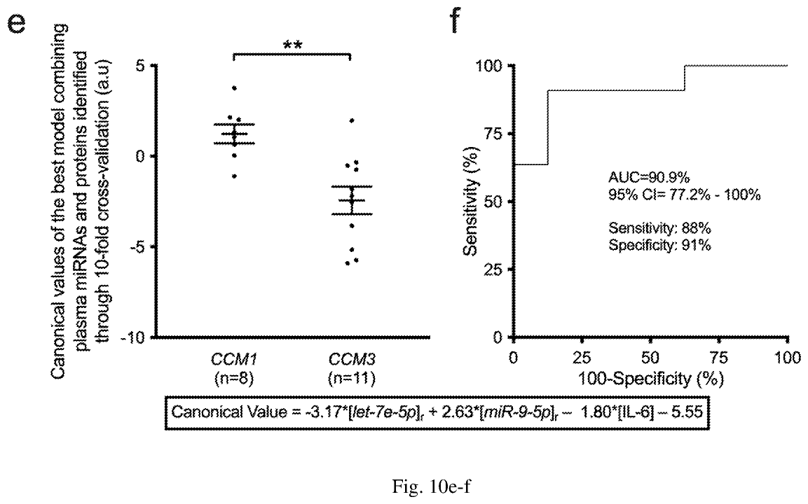

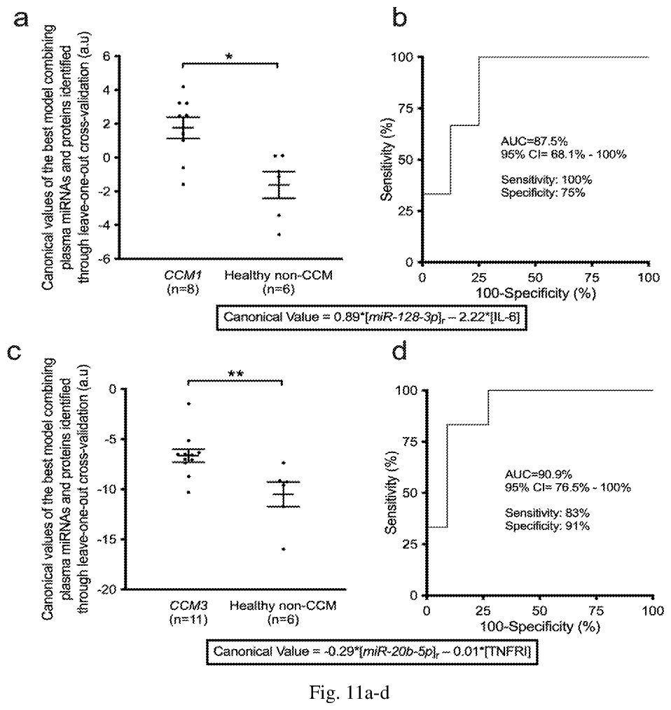

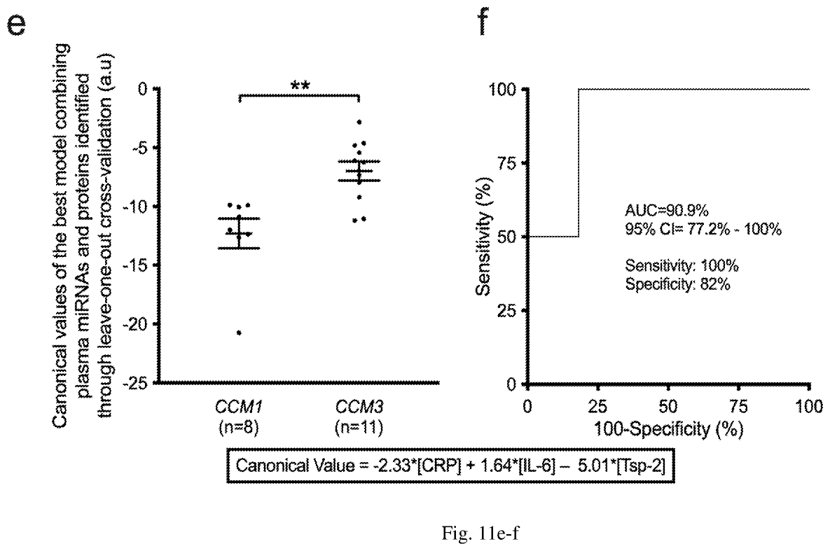

A three-step Bayesian-informed machine learning (ML) approach was implemented to assess if a weighted combination of plasma levels of miRNAs (i.e., assessed by RT-qPCR), and proteins with a documented role in CCM disease (i.e., assessed by ELISA) shows better performance to distinguish familial-CCM. This Bayesian paradigm relies on prior information, such as the biological relevance of candidate molecules, to inform biomarker development [49]. Additionally, ML approaches allow for the combination of the best performing plasma molecules, given that individual molecules may have imperfect sensitivity/specificity [7]. A thorough literature search was conducted to identify 21 plasma proteins related to angiogenesis, endothelial permeability, inflammation, immune response, and extracellular matrix remodeling, with reported associations to CCM or neurovascular diseases [7, 26, 50, 51]. As a proof of concept, the inventors assessed familial disease reflecting germline heterozygosity of any CCM gene, since the sample sizes of specific CCM genotypes were insufficient for separate statistical analyses (FIG. 10-11). Two different approaches were developed using 10-fold and leave-one out cross validation to first select the best individual proteins and miRNAs which reflected familial-CCM disease (FIG. 6). These cross validations reduce the overfitting effects on a limited dataset, while excluding any molecules which would not individually contribute to the model's diagnostic association [52]. Selected miRNAs or proteins were then used to create the best models including (1) only plasma levels of miRNA, (2) only plasma levels of proteins, and finally (3) combining miRNAs and proteins to diagnose familial-CCM (FIG. 6). In each case, the best model was identified as having the lowest Akaike information criterion (AIC) score. Feature selection minimized the dependency among features under the assumption of logistic regression, while selecting the model with AIC scores reduced the overall complexity of the model to prevent overfitting. Finally, all three models were tested on the same cohort of patients to compare performance through sensitivity and specificity (FIG. 6). Refer to supplemental material for additional Methods.

### B. Results

1. DE miRNAs in Peripheral Blood Plasma of Ccm1 and Ccm3 Murine Models

Differential expression analyses identified 17 DE miRNAs in the plasma of Ccm1—mice (Table S1), and 44 in Ccm1^{+/-} mice [all: p<0.1, FDR corrected] (Table S2) when compared to their respective wild type mice. In the Ccm3 models, 142 plasma miRNAs were DE in the homozygous mice (Table S3), and 9 in the heterozygous mice when compared to their respective wild type mice [all: p<0.1, FDR corrected] (Table S4). Finally, 99 DE plasma miRNAs were identified between Ccm1^{-/-} and Ccm3^{-/-} mice [all: p<0.1, FDR corrected] (Table S5). No plasma miRNAs were DE between Ccm1^{+/-} and Ccm3^{+/-} mice. Non-sensitized Ccm1 and Ccm3 heterozygous mice have been shown to harbor a negligible lesion burden. Thus, these DE miRNAs were discovered to reflect disease genotype, rather than disease severity.

2. Homologous Mouse-Human DE miRNAs in Peripheral Blood Plasma of Patients with CCM1 and CCM3 Disease

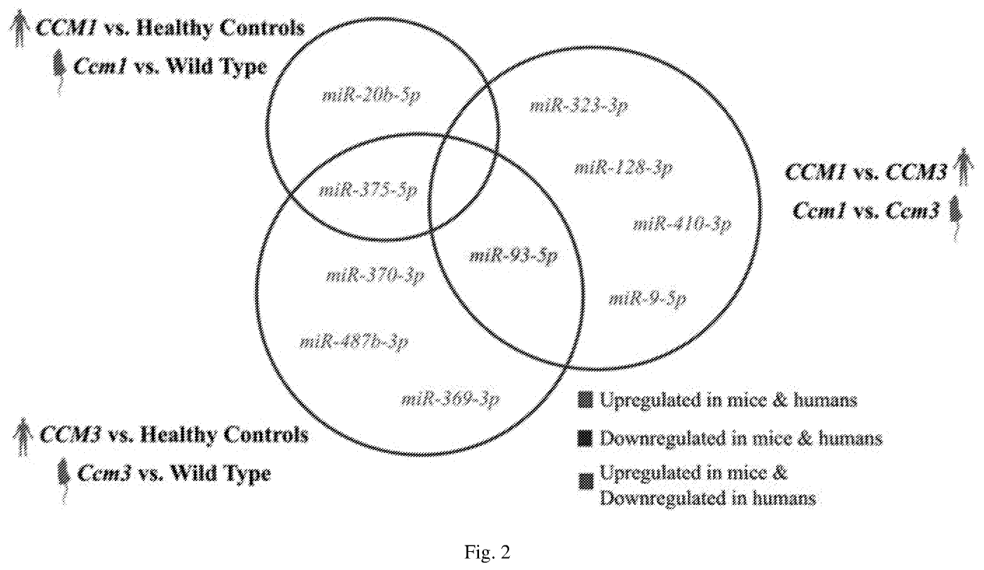

Differential analyses identified 16 DE plasma miRNAs between familial-CCM1 patients and healthy non-CCM controls [p<0.05] (Table S6). The PLS-DA results showed that the DE miRNAs (p<0.05) identified through RNA-seq are able to distinguish CCM1 from healthy non-CCM subjects (top 2 components, both: AUC 100%, p=7.5×10⁻⁴). Two of these 16 miRNAs, miR-20b-5p [fold change (FC)=25.9] and miR-375-3p [FC=3.5] had their murine homolog also DE in the plasma of Ccm1^{+/-} mice [FC=2.6, 5.2 respectively; both: p<0.1, FDR corrected] (FIG. 2). No plasma miRNAs were commonly DE between familial-CCM1 patients and Ccm1′ mice.

In the familial-CCM3 patients, 12 DE plasma miRNAs were identified compared to healthy non-CCM controls [p<0.05] (Table S7). PLS-DA results confirmed that DE miRNAs (p<0.05) can differentiate CCM3 from healthy non-CCM subjects (top 2 components, both: AUC 100%, p=6.4×10⁴). Five of these 12 DE plasma miRNAs, miR-93-5p [FC=0.6], miR-370-3p [FC-4.3], miR-487b-3p [FC=13.3], miR-369-5p [FC=19.2], and miR-375-3p [FC=2.8] had their murine homolog also DE in the plasma of Ccm3^{+/-} mice [FC=0.5, 2.7, 6.7, 2.7, 3.5 respectively; all: p<0.1, FDR corrected] (FIG. 2). No plasma miRNAs were commonly DE between familial-CCM3 patients and Ccm3+/mice. It is unclear why homologous DE miRNAs associated with germline CCM1 and CCM3 heterozygosity in humans were shared with heterozygous Ccm1+/mice and homozygous Ccm3˜/mice, respectively. The number of plasma DE miRNAs in heterozygous Ccm3^{+/-} and postnatally induced Ccm1^{-/-} mice was low, which may have been due to a low read depth during sequencing. In addition, postnatally induced gene loss in mouse models probably reflects an acute phase of the pathogenesis of CCMs, and not the chronic aspect seen in the human disease; these differences are exaggerated in the more aggressive Ccm3 models.

The inventors further identified 24 plasma DE miRNAs between CCM1 and CCM3 patients [p<0.05] (Table S8). DE miRNAs (p<0.05) identified through RNA-seq were shown to distinguish CCM1 from CCM3 through an additional PLS-DA (component 1, AUC 98.15%, p=2.2×10⁴; component 2, AUC 100%, p=1.2×10⁴). Of these, miR-128-3p [FC=0.6], miR-410-3p [FC=2.0], miR-9-5p [FC=0.07], miR-323b-3p [FC=13.7], and miR-93-5p [FC-0.7] had their murine homolog also DE between Ccm1^{-/-} and Ccm3^{-/-} murine models [FC=1.8, 1.8, 28.2, 6.6, and 0.6 respectively; all: p<0.1, FDR corrected] (FIG. 2).

3. Gene Targets of Homologous Mouse-Human DE Plasma miRNAs

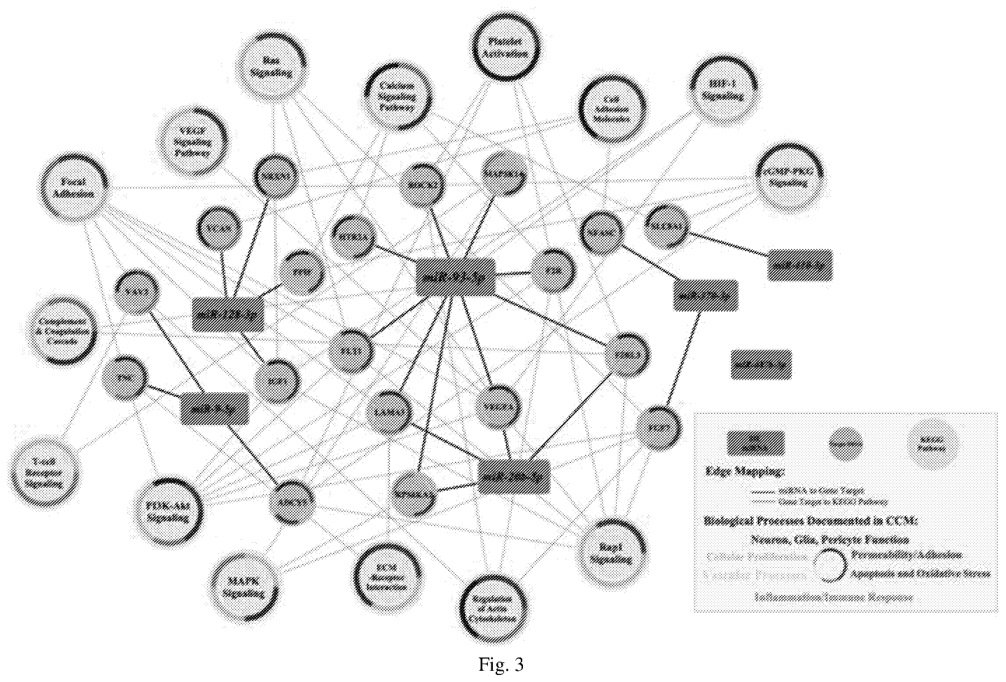

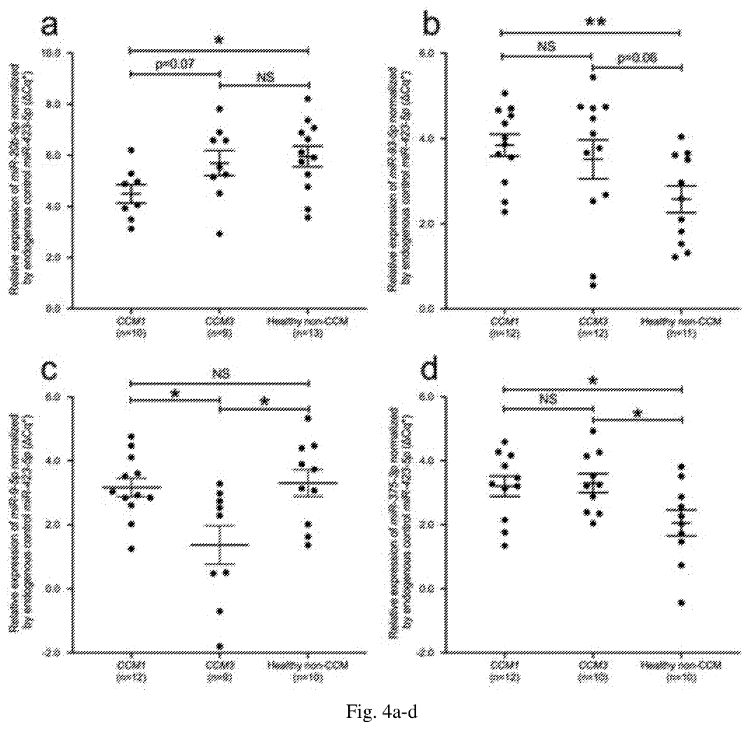

Of the 10 homologous DE miRNAs, 8 were DE in the same direction in familial-CCM patients and preclinical mouse models (all genotypes) (FIG. 2). Seven homologous DE miRNAs, targeting a range of 0.1%-2.4% of the total human genome, had DEG targets within the published transcriptome of laser micro-dissected NVUs of human surgically resected CCMs (FIG. 3). In addition, 5 of those 7 miRNAs also had DEG targets within either the transcriptome of (1) in vitro Ccm1^{-/-} or Ccm3^{-/-} BMECs, or (2) laser micro-dissected lesional NVUs of Ccm3^{+/-} mice. To further classify these gene interactions, the KEGG was used to generate enriched KEGG pathways [p<0.05, FDR corrected; Bayes factor≥3] for each CCM transcriptome (data not shown).

DE miR-20b-5p, which was increased in the plasma of familial-CCM1 patients, and miR-93-5p, which was decreased in familial-CCM3 patients compared to healthy non-CCM controls, respectively had 8.9% and 9.1% of their gene targets within the transcriptome of human micro-dissected CCM lesional NVUs. CCM-enriched KEGG pathway analyses showed that these DEGs were associated with PI3K-Akt signaling, focal adhesion, HIF-1, Rap1 signaling, and other pathways involved in CCM mechanisms [all: p<0.05, FDR corrected] (data not shown). Specifically, both miR-20b-5p and miR-93-5p target VEGFA and ADAMTS5. Additionally, miR-93-5p targets ROCK2 and MAP3K14, which are associated with CCM pathogenesis. miR-93-5p provides a unique translational opportunity for biomarker development as it also targets Vegfa in mice, which is a DEG in the transcriptome of laser micro-dissected lesional NVUs of Ccm3^{+/-} mice.

DE miR-370-3p and miR-487b-3p, both found to be increased in the plasma of familial-CCM3 patients, were also found to target several DEGs in the transcriptome of human micro-dissected CCM lesional NVUs. Specifically, 9.4% of miR-370-3p's gene targets, and 33.3% of miR-487b-3p's targets were within the human CCM lesional transcriptome. miR-370-3p targets NFASC and FGF7, which were both found to be associated with PI3K-Akt signaling, Rap1 signaling, and cell adhesion molecules [all: p<0.05, FDR corrected] (data not shown). It also targets Klhl21, a DEG in the transcriptome of micro-dissected lesional NVUs of Ccm3^{+/-} mice. In addition, miR-487b-3p was found to target NRARP, which is DE in the human CCM lesional transcriptome.

Finally, DE miR-128-3p and miR-9-5p, which were decreased, and miR-410-3p, which was increased (i.e., all between familial-CCM1 compared to familial-CCM3 patients) also target DEGs within the human CCM lesional transcriptome. miR-128-3p, miR-9-5p, and miR-410-3p had 10.8%, 13.5%, and 9.0% of their gene targets in the human CCM lesional transcriptome, respectively. Among these, IGF1 and NRXN1 are targeted by mir-128-3p. Of interest, IGF1 is associated with enriched-KEGG pathways such as PI3K-Akt signaling, HIF-1 signaling, and Rap1 signaling, while NRXN1 is related to cell adhesion molecules [all: p<0.05, FDR corrected] (data not shown). Also, miR-128-3p targets Foxq1 in mice, which is DE in the transcriptome of Ccm1^{-/-} mouse BMECs. Furthermore, miR-9-5p targets TNC, VAV3, and VCAN, which are associated with focal adhesion and cell adhesion KEGG pathways [all: p<0.05, FDR corrected] (data not shown). Additionally, miR-9-5p targets Rap1b which is DE in the transcriptome of Ccm1^{-/-} mouse BMECs. Finally, miR-410-3p targets two DEGs within the human CCM lesional transcriptome, both of which are part of the solute carrier group of membrane transport proteins. Interestingly, one of these genes, SLC8A1, is also targeted by miR-410-3p in mice and is a DEG in the transcriptome of laser micro-dissected lesional NVUs of Ccm3^{+/-} mice.

4. Plasma miRNA as Biomarkers of Patients with New Lesion Formation

Ninety-seven plasma miRNAs [p<0.05, non-FDR corrected] were DE in familial-CCM patients that developed a new lesion within a year after their blood draw, as compared to those with stable lesion burden (Table S13). Fifteen of these 97 were also DE in familial-CCM3 patients, when compared to healthy non-CCM subjects or familial-CCM1 patients. Of these 15 plasma miRNAs, 9 (miR-141-3p, miR-409-3p, miR-431-5p, miR-485-3p, miR-9-5p, miR-128-3p, miR-379-5p, miR-205-5p, and miR-1271-5p) were found to target DEGs within the transcriptome of laser micro-dissected NVUs of human surgically resected CCMs. These DEGs include ROCK2, VEGFA, ADAMTS5, VCAN, and NRXN1, which are associated with several CCM-enriched KEGG pathways such as PI3K-Akt signaling, HIF-1 signaling, Rap1 signaling, focal adhesion, and cell adhesion molecules [all: p<0.05, FDR corrected] (data not shown).

5. Peripheral Blood Assays of DE miRNAs as Biomarkers of CCM Genotype

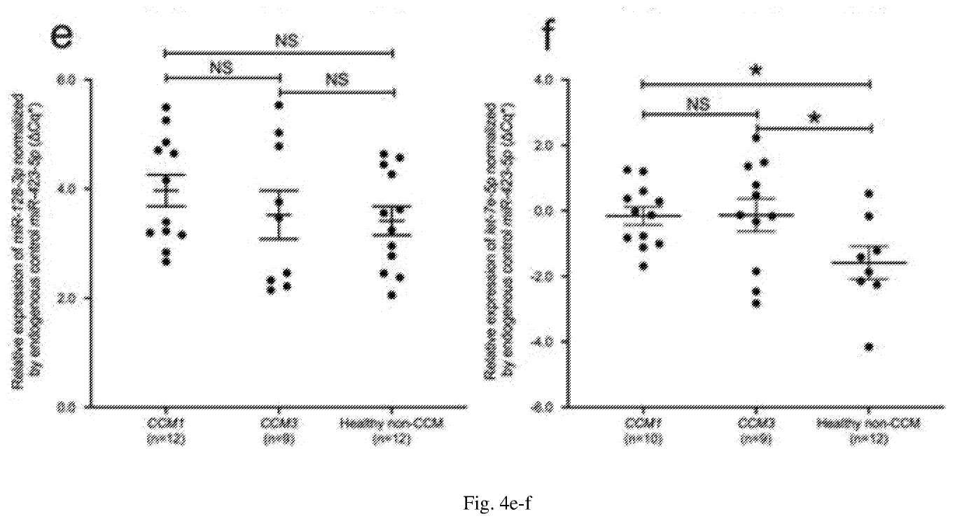

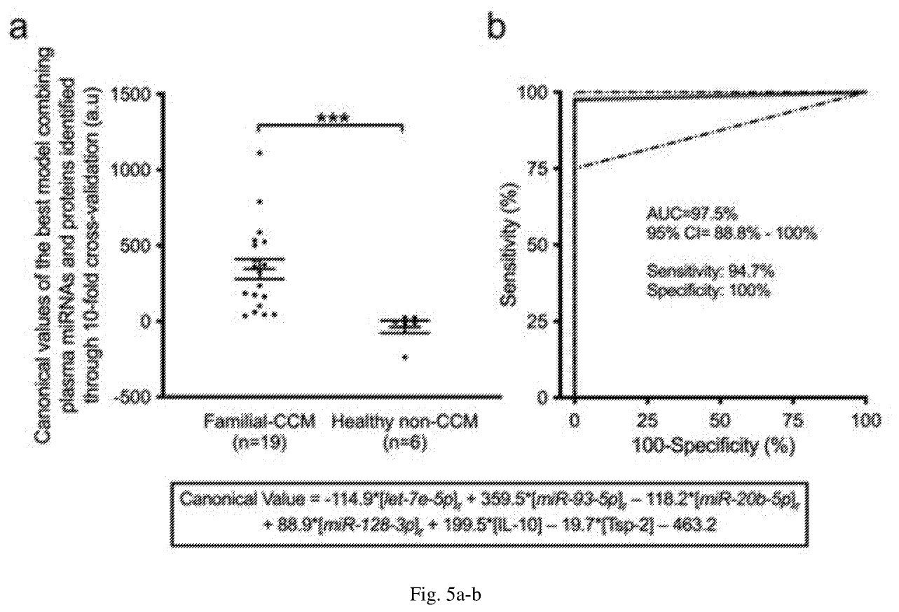

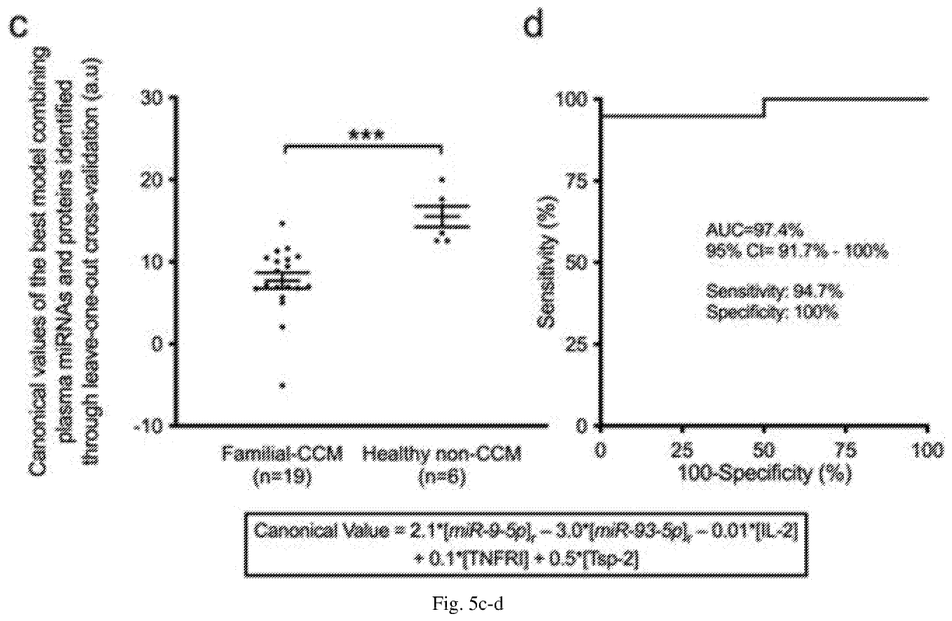

A validation of the miRNA sequencing differential analyses was performed by assessing the plasma levels of a selected panel of six DE miRNAs using RT-qPCR within an independent cohort of 12 familial-CCM3, 12 familial-CCM1 patients, and 13 healthy non-CCM controls (FIG. 4, FIG. 6). Relative quantification levels assessed through RT-qPCR supported the directionality and dysregulation observed in the miRNome sequencing data.

Lower RT-qPCR relative quantification values of miR-20b-5p in the plasma of familial-CCM1 were observed compared to healthy non-CCM controls [p=0.04] (FIG. 4a). A trend toward lower relative quantification values of miR-20b-5p was also observed in the plasma of familial-CCM1 patients compared to familial-CCM3 patients [p=0.07] (FIG. 4a). No difference was observed between familial-CCM3 patients and healthy non-CCM subjects.

Familial-CCM1 patients also showed greater RT-qPCR relative quantification values of plasma miR-93-5p compared to healthy non-CCM subjects [p=0.007] (FIG. 4b). In addition, a trend toward higher relative quantification values was observed between familial-CCM3 compared to healthy non-CCM subjects [p=0.06] (FIG. 4b). No difference between familial-CCM3 and familial-CCM1 patients was observed.

The relative plasma quantification RT-qPCR values of miR-9-5p were higher in the plasma of familial-CCM1 patients [p=0.01] as well as in healthy non-CCM subjects compared to familial-CCM3 patients [p=0.01] (FIG. 4c). The relative quantification values of miR-375-3p were also higher in the plasma of familial-CCM1 [p=0.05] and familial-CCM3 patients [p=0.02] compared to healthy non-CCM subjects (FIG. 4d).

Finally, the relative quantification plasma values for miR-128-3p did not show differences in any of the comparisons (FIG. 4e), while relative quantification values of the miRNA let-7e-5p were higher in the plasma of familial-CCM1 compared to healthy non-CCM subjects [p=0.01] (FIG. 4f).

Further linear Pearson correlation analyses showed no correlation between relative quantification of any of these miRNAs and the lesion burden. These results suggest that these DE plasma miRNAs reflect disease genotype, rather than severity.

6. Integration of Plasma Levels of DE miRNAs with Proteins Enhances their Association with Familial-CCM

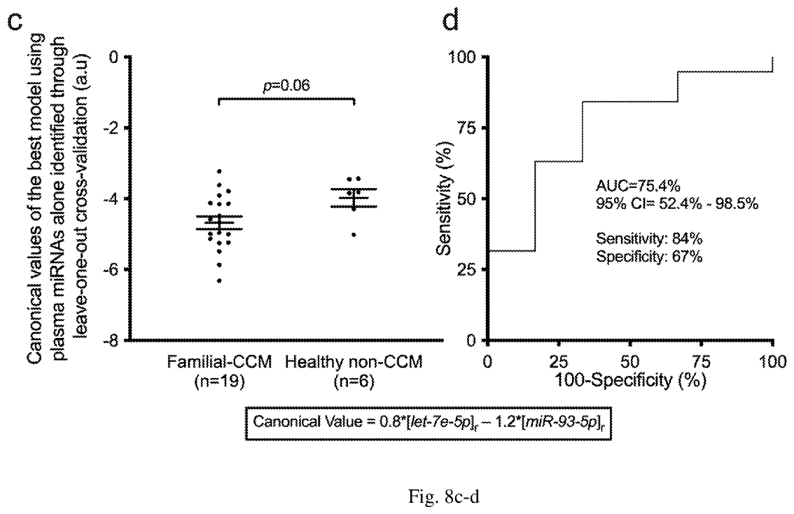

The relative quantification plasma levels of the six miRNAs were used to create the most optimal weighted combination (i.e., achieving the lowest AIC score) able to diagnose familial-CCM patients in comparison to non-CCM controls (n=24). This model's accuracy was defined as fair in distinguishing familial-CCM patients with 84% sensitivity and 67% specificity [area under the ROC curve (AUC)=75.4%, CI=52.4%-98.5%] (FIG. 8).

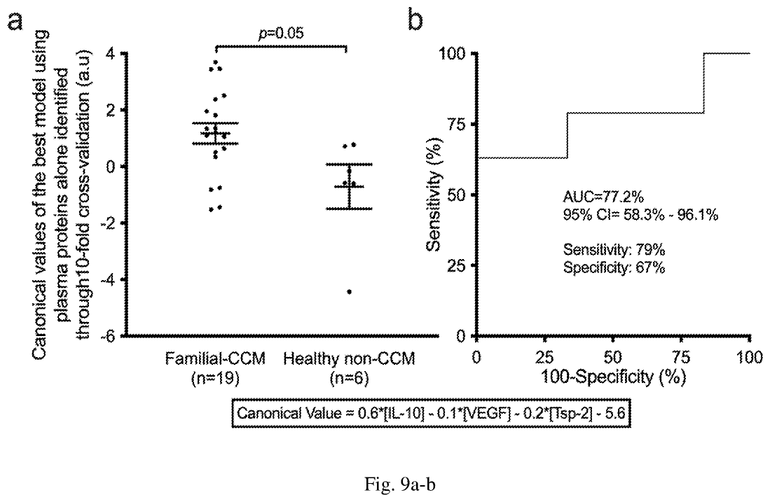

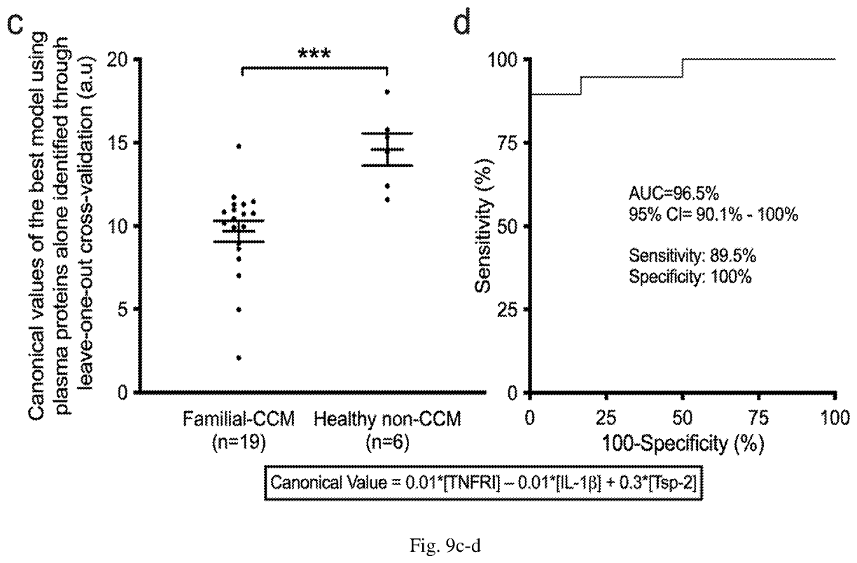

A similar approach was performed using plasma levels of proteins in familial-CCM patients (n=46). Using two independent ML approaches, the best weighted combination of plasma proteins identified familial-CCM patients from healthy non-CCM with sensitivity up to 89.5% and specificity up to 100% [optimal AUC=96.5%, CI=90.1%-100%] (FIG. 9).

Given the imperfect diagnostic performance of miRNAs or proteins alone, combining the two may offer a more holistic model reflecting various aspects of the disease. Thus, an integrated biomarker including proteins and miRNAs was developed using familial-CCM patients (n=19) with both plasma levels of protein and relative quantification of miRNAs. Using a 10-fold cross validation ML approach, the weighted combination of proteins and relative quantification levels of miRNAs diagnosed familial-CCM patients with an accuracy of 95%, with a sensitivity of 94.7% and sensitivity of 100%. (FIGS. 5a and b):

$$Canonical Value = - 114.88 * {[ let - 7 e - 5 p ]}_{r} + 359.47 * {[ miR - 93 - 5 p ]}_{r} - 118.16 * {[ miR - 20 b - 5 p ]}_{r} + 88.93 * {[ miR - 128 - 3 p ]}_{r} + 199.47 * [ IL - 10 ] - 19.7 * [ Tsp - 2 ] - 463.18$$

with [X]ᵣ denoting relative quantification of miRNAs. This model estimated higher canonical values for familial-CCM patients compared to non-CCM healthy controls [p<0.0001] (FIG. 5a).

In order to validate these results, another ML approach, a leave-one-out cross validation, was also applied. This approach yielded a combination with the same performance of 94.7% sensitivity and 100% specificity (FIGS. 5c and d) in diagnosing familial-CCM, however it included less compounds making it potentially more feasible for future clinical applicability:

$$Canonical Value = 2.05 * {[ miR - 9 - 5 p ]}_{r} - 3.02 * {[ miR - 93 - 5 p ]}_{r} - 0.01 * [ IL - 2 ] + 0.1 * [ TNFRI ] + 0.46 * [ Tsp - 2 ]$$

This additional weighted combination calculated lower canonical scores for familial-CCM patients compared to non-CCM healthy controls [p<0.0001] (FIG. 5c). Refer to supplemental material for additional results.

### C. Supplementary Methods

Murine and Human CCM Transcriptomes: The transcriptomes of in vitro Ccm1^{-/-} and Ccm3/BMECs, in vivo Ccm3^{+/-} murine lesional CCMs, and neurovascular units (NVUs) from human lesional tissue were all previously published and were used here for putative target and KEGG pathway correlation of DE miRNAs. Methods for collection of tissue and construction of transcriptomic data is clearly delineated within these previously published articles. [1-3]

Processing of plasma from familial-CCM patients: For each patient, the blood sample was collected at the time of their clinic visit. Standard clinical 10-ml heparinized Vacutainer tubes (BD Vacutainer, Becton, Dickinson and Company) were used to draw blood samples. The plasma was isolated by centrifugation at 500 g at 4° C. for 10 minutes (AllegraX-30R, Beckman Coulter). Subsequently, 300 μl of plasma was aliquoted into 1.7-ml microcentrifuge tubes and stored at −80° C. [4-6].

Plasma miRNome sequencing: Following RNA extraction using the miRNeasy Serum/Plasma Kit (Qiagen), cDNA libraries were generated with commercially available Illumina small RNA-Seq kits and sequenced using the Illumina HiSeq 4000 platform (Illumina) with single-end 50-bp reads. Fast QC (v0.11.5) was used to assess raw sequencing quality (found on the world wide web at bioinformatics.babraham.ac.uk/projects/fastqc/).

The small RNA adapter sequences were trimmed from small RNA sequencing data with cutadapt (found online at cutadapt.readthedocs.io). The trimmed reads were then mapped and quantified either to the human mature miRNA database for human samples, or the mouse mature miRNA database for mouse samples (miRbase, found on the world wide web at mirbase.org/) using sRNAbench library (found online at bioinfo2.ugr.es/ceUGR/srnabench/) mapping strategy [7] and wrapped bowtie alignment (with alignment type=−n, seed length for alignment=20, minimum read-count=2, allowed number of mismatch=0, minimum read count=2, and maximum number of multiple mappings=20). MiRNAs with low expression were removed before further downstream analyses. Finally, the miRNA expression values were normalized following trimmed mean of M-values normalization methods with library size correction [2, 6].

Plasma proteins with a documented role in CCM disease: A panel of 21 plasma proteins which had previously elucidated roles in CCM (Table S2) were measured using the enzyme-linked immunosorbent assay (ng/ml) and Multiplex (pg/ml) assay in accordance with manufacturer protocols in 46 CCM patients and 28 non-CCM controls.

The plasma levels of thrombomodulin (TM), thrombospondin-1 (Tsp-1), thrombospondin-2 (Tsp-2) were assessed using commercially available ELISA assays kits (R&D Systems, Minneapolis, Minnesota, USA). All plates were washed with a BioTek 405TS plate washer (BioTek Instruments, Winooski, VT, USA), and absorbances measured using Bio-Rad iMark plate reader (Bio-Rad, Hercules, CA, USA). The assays were performed in the Neurovascular Research team laboratory at the University of Chicago. In each plate, the plasma samples were loaded in parallel duplicate wells, and then averaged. Fifty beads per region were collected for each well, and a 5-parameter logistic regression analysis was performed to estimate the sample concentration.

The plasma levels of tumor necrosis factor alpha (TNFα), tumor necrosis factor receptor I (TNFRI), matrix metalloproteinase-2 (MMP2) and −9 (MMP9), chemokine ligand 2 (CCL2/MCP1), soluble endoglin/CD105 (sENG), soluble vascular cell adhesion protein 1 (sVCAM1), soluble intercellular adhesion molecule 1 (sICAM1/CD54), interleukin-1 beta (IL-1β), IL-2, IL-6, IL-8/CXCL-8, IL-10, vascular endothelial growth factor (VEGF), soluble roundabout guidance receptor 4 (sROBO4), interferon gamma (IFNγ), soluble cluster of differentiation 14 (sCD14), and C-reactive protein (CRP) were assessed using 5 customized magnetic bead-based multiplex Luminex screening immunoassay kits (R&D Systems, Minneapolis, Minnesota, USA) [8, 9]. The measurements were performed with a BioRad BioPlex-100 analyzer (Bio-Rad Laboratories, Hercules, California, USA) running the BioPlex Manager Software version 5.0, or the Luminex 200 System (Luminex Corporation, Austin, Texas, USA) running with xPONENT Software. The assessment was performed at the Flow Cytometry Core Facility at the University of Chicago. In each plate, the plasma samples were loaded in parallel duplicate wells, and then averaged. Plasma values greater±3 standard deviations from the mean were excluded as outliers.

Univariate Statistics and Batch correction for protein level assessment: Differences in relative and absolute levels of miRNAs measured by RT-qPCR, were analyzed using a Mann-Whitney test. Canonical values of the best weighted combinations developed during the three-step integrative approach were calculated for each patient and compared between familial-CCM patients and healthy-non-CCM subjects using an unpaired two sample Student's t-test, assessed with pooled standard deviation, or Mann-Whitney test according to the equality of the variance. All statistical analyses were performed on Prism (GraphPad, San Diego, California, USA). All p-values were considered statistically significant at p<0.05.

Variation due to non-biological experimental variations is common when utilizing multiplex or microarray analysis [10]. A principal component analysis revealed significant variation between batches in the plasma levels of the molecules. Thus, batch effects were corrected using the Combat function from the R package SVA [11].

Integrated plasma miRNA and protein biomarker development: A three-step integrative approach was developed to define and compare the best weighted models of circulating plasma compounds (FIG. 6). The three models generated were a weighted combination of (1) only relative quantification of plasma miRNAs (Familial-CCM patients, n=24; Healthy non-CCM, n=13), (2) only plasma levels of proteins with a documented role in CCM disease (Familial-CCM patients, n=46; Healthy non-CCM, n=28), (3) both relative quantification of plasma miRNAs and proteins (Familial-CCM patients, n=19; Healthy non-CCM, n=6).

The selection of plasma compounds with higher individual predictive ability, and lower degree of redundancy was performed using machine learning cross-validation (CV). Two CV approaches, (1) 10-fold CV and (2) leave-one-out CV, were independently performed, creating two different sets of candidate compound biomarkers of (1) miRNAs alone, (2) proteins alone, and (3) a combined integrative model. The features selected at least once during the CV process were included in the initial set of candidate compound biomarkers to be used for model selection. The overall accuracy, specificity, sensitivity, receiver operating characteristic (ROC) curve were assessed using the Youden index [12].

A machine learning framework was used, consisting of a correlation-based feature selection, a logistic linear regression, and a tree-based model selection. A 10-fold CV was enforced throughout the framework to reduce the potential overfitting effects on the limited dataset. The logistic regression model with ridge penalty was applied as the classifier. This was given as a binary classification on n samples with m features. Probability for the positive class for any sample i was estimated using:

$$P ( {X}_{i} ) = \frac{{e}^{{X}_{i} β}}{{e}^{{X}_{i} β} + 1}$$

where Xᵢ is the feature vector for sample i and β is the vector of the m coefficients. The negative multinomial log-likelihood was minimized according to the following:

$$L = - {\sum }_{i = 1}^{n} ( {Y}_{i} ln p ( {X}_{i} ) + ( 1 - {Y}_{i} ) ( 1 - ln p ( {X}_{i} ) ) + ridge * {β}^{2}$$

where Yᵢ is the label for sample i and ridge parameter=1.0×10⁻⁸.

After the feature selection, a breadth-first tree search was performed with all candidate compound biomarkers in order to assess all the possible combinations. The most optimal combination was selected as minimizing the Akaike information criterion (AIC) score calculated using the mean absolute error from the logistic regression classifier as the predictive power, and the number of features k as the complexity of the model. The final logistic regression coefficients for the best model were then defined on all the patients with both miRNAs and proteins.

The leave-one-out CV was performed with multivariable logistic regression using feature selection through the glmnet library implemented in R program (Miami, Florida, USA) [13]. During each CV cycle, one subject was left as the independent testing sample, while the remaining subjects were enrolled as training samples for multivariate modeling. The best model with the lowest AIC score was selected similarly to the 10-fold CV.

Literature review for CCM-relevant biological processes: In order to identify a comprehensive list of CCM-related publications, a systematic electronic review was performed querying the PubMed database of peer-reviewed articles published between January 2011 and April 2021 using the following key terms: (cerebral cavernous malformation [Title/Abstract] OR cerebral cavernous malformations [Title/Abstract] OR Cavernous Malformations [Title/Abstract] OR cerebral vascular malformation [Title/Abstract] OR cerebrovascular malformation [Title/Abstract] OR cavernous angioma [Title/Abstract] OR CCM1 [Title/Abstract] OR CCM2 [Title/Abstract] OR CCM3 [Title/Abstract] OR Krit1 [Title/Abstract] OR PDCD10 [Title/Abstract] OR MGC4607 [Title/Abstract]) AND (english [Language]) NOT (case report [Publication Type] NOT liver [Title/Abstract] NOT surgery [Title/Abstract] NOT management [Title/Abstract]) NOT treatment [Title/Abstract]).

This search yielded a total of 1074 abstract, of which, 127 reported mechanistic pathways associated to the physiopathogenesis of CA disease. The list of KEGG pathways enriched within the human CMM lesional transcriptome and related to differently expressed genes (DEGs) targeted by DE miRNAs of interest were queried within these 127 abstracts for biological functions. Based on this search, KEGG pathways were classified into 6 previously published CCM-related biological processes [1, 2], including: cellular proliferation, inflammation and immune response, permeability and adhesion, neuron, glia and pericyte functions, apoptosis and oxidative stress, and vascular processes. Target DEGs of DE miRNAs were also linked to these 6 CCM processes based on the KEGG pathways they were found to be associated with.

### D. Supplementary Results

### 1. CCM-Enriched KEGG Pathways

To further classify gene interactions, the Kyoto Encyclopedia of Genes and Genomes (KEGG) was used to independently generate enriched-KEGG pathways (p<0.05, FDR corrected; Bayes factor≥3) using the DEGs (p<0.05, FDR corrected) within the transcriptome of (1) laser micro-dissected NVUs of human surgically resected CCMs, (2) in-vitro Ccm1^{-/-}, (3) Ccm3^{-/-} mouse brain microvascular endothelial cells (BMECs), or (4) laser micro-dissected lesional NVUs of Ccm3^{+/-} mice. Eighty-five enriched-KEGG pathways were identified in the transcriptome of laser micro-dissected NVUs of human surgically resected CCMs (data not shown); 130 in the transcriptome of mouse in vitro Ccm1^{-/-} BMECs (data not shown); 23 in the transcriptome of mouse in vitro Ccm3^{-/-} BMEC (data not shown), and 5 in the transcriptome of mouse in vivo Ccm3^{+/-} lesional NVUs (data not shown).

2. Diagnostic Biomarkers Using Plasma miRNAs and Proteins Independently

Both the 10-fold and leave-one-out CV did not exclude any of the 6 miRNAs tested for individual diagnostic contribution as they all had non-zero coefficients (i.e., CV coefficients>0), and thus all were considered as possible variables during model selection. However, the relative quantification was selected over absolute quantification as it showed higher sensitivity and specificity when tested for diagnostic association with familial-CCM.

The 10-fold CV selected 7 plasma proteins to be used as candidate compound biomarkers, including CRP, IL-10, MMP-9, TNFRI, VEGF, ROBO4, and Tsp-2 (all: CV coefficients>0). While the leave-one-out CV selected 9 proteins: CCL2, sCD14, IL-2, IL-6, MMP-9, TNFRI, VEGF, ICAM1, Tsp-2.

The best weighted combination using only the plasma relative quantification of miRNAs assessed by the 10-fold cross-validation was able to distinguish familial-CCM patients with 68% accuracy, performing with 84% sensitivity and 67% specificity when tested on the full dataset (FIGS. 8a and b):

$$Canonical Value = - 0.72 * {[ let - 7 e - 5 p ]}_{r} + 1.07 * {[ miR - 93 - 5 p ]}_{r} - 3.94$$

with [X]ᵣ denoting relative quantification of miRNAs

Canonical values estimated with this model showed a higher trend in familial-CCM patients compared to healthy non-CCM subjects (p=0.06; FIG. 8a).

The leave one out cross-validation approach defined a similar weighted combination able to distinguish familial-CCM patients with 84% sensitivity and 67% specificity (FIG. 8c & d), and included:

$$Canonical Value = 0.82 * {[ let - 7 e - 5 p ]}_{r} - 1.21 * {[ miR - 93 - 5 p ]}_{r}$$

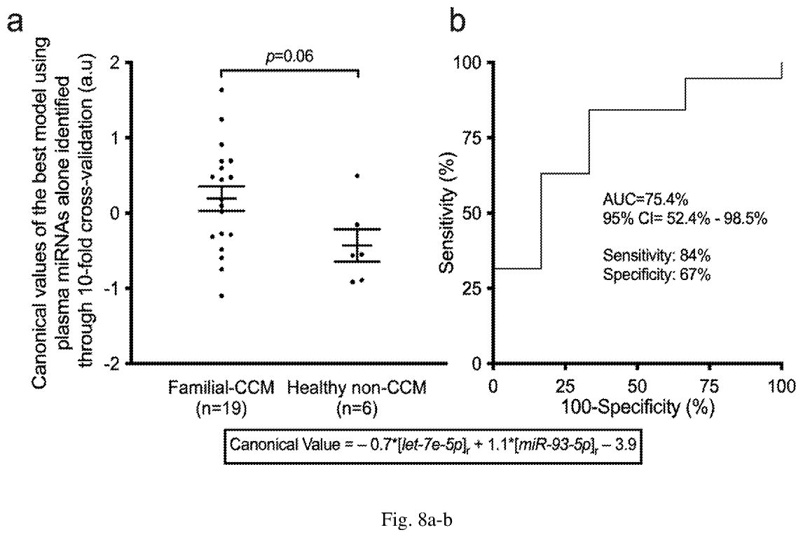

The canonical values calculated with this weighted combination also showed a lower trend (p=0.06) in familial-CCM patients compared to healthy non-CCM subjects (FIG. 7c).

The same approach was implemented to compute a weighted combination using plasma levels of proteins only assessed by 10-fold cross-validation. The model generated was able to diagnose familial-CCM patients with 79% sensitivity and 67% specificity on the full dataset, with an accuracy of 68% during CV (FIG. 9a & b), and included:

$$Canonical Value = 0.59 * [ IL - 10 ] - 0.1 * [ VEGF ] - 0.15 * [ Tsp - 2 ] - 5.97$$

Canonical values of familial-CCM patients were higher (p=0.05) compared to healthy non-CCM subjects (FIG. 9a).

The leave-one-out cross validation pipeline selected a weighted combination of proteins able to diagnose familial-CCM patients with 89.5% sensitivity and 100% specificity (FIGS. 9c and d), and included:

$$Canonical Value = 0.01 * [ TNFRI ] - 0.01 * [ IL - 2 ] - 0.29 * [ Tsp - 2 ]$$

Finally, the canonical values were lower (p<0.0001) in familial-CCM patients compared to healthy non-CCM subjects (FIG. 9c).

Diagnostic biomarkers of specific genotype using combined plasma miRNAs and proteins

A similar three-step integrative approach was also applied to create the best diagnostic biomarkers for specific familial-CCM1 and familial-CCM3 genotypes using both plasma miRNAs and proteins.

10-Fold Cross Validation: The best weighted combination using plasma miRNAs and proteins was able to distinguish familial-CCM1 patients from non-CCM healthy controls with 71% accuracy when tested using CV, and performed with 100% sensitivity and 83% specificity on the full dataset (FIGS. 10a and b):

$$Canonical Value = - 2.18 * [ let - 7 e - 5 p ] r + 2.87 * [ miR - 93 - 5 p ] r + 3.67 * [ TM ] - 23.04$$

Higher canonical values (p=0.01) were calculated in familial-CCM1 patients compared to healthy non-CCM subjects (FIG. 10a).

The same approach led to weighted combination able to distinguish familial-CCM3 patients from non-CCM healthy controls with 100% sensitivity and 100% specificity on the full dataset, and 88% accuracy during CV (FIGS. 10c and d):

$$Canonical Value = 38.88 * [ miR - 93 - 5 p ] r - 13.18 * [ miR - 128 - 3 p ] r - 22.54 * [ miR - 375 - 3 p ] r + 0.33 * [ CCL 2 ] - 0.22 * [ TNFRI ] - 6.51 * [ Tsp - 2 ] + 186.92$$

Again, higher canonical values were higher (p=0.0002) in familial-CCM3 patients compared to healthy non-CCM subjects (FIG. 10c).

Finally, the weighted combination using plasma miRNAs and proteins was able to distinguish familial-CCM1 from familial-CCM3 patients with 88% sensitivity and 91% specificity when tested on the full dataset, and 79% accuracy during CV (FIGS. 10e and f):

$$Canonical Value = - 3.17 * [ let - 7 e - 5 p ] r + 2.63 * [ miR - 9 - 5 p ] r - 1.8 * [ IL - 6 ] - 5.55$$

Canonical values estimated with this model were higher (p=0.0018) in familial-CCM1 patients compared to CCM3 patients (FIG. 10e).

Leave-one-out Cross Validation: The best weighted combination using plasma miRNAs and proteins assessed by leave-one-out cross-validation was able to distinguish familial-CCM1 patients from non-CCM healthy controls with 100% sensitivity and 75% specificity (FIGS. 11a and b), and included:

$$Canonical Value = 0.89 * {[ miR - 128 - 3 p ]}_{r} - 2.22 * [ IL - 6 ]$$

Familial-CCM1 patients had higher canonical values (p=0.01) than healthy non-CCM subjects (FIG. 11a).

A similar approach using plasma miRNAs and proteins computed a weighted combination able to distinguish familial-CCM3 patients from non-CCM healthy controls with 83% sensitivity and 91% specificity (FIGS. 11c and d):

$$Canonical Value = - 0.29 * {[ miR - 20 b - 5 p ]}_{r} - 0.01 * [ TNFRI ]$$

Canonical values estimated with this model were higher in familial-CCM3 patients (p=0.0048) compared to healthy non-CCM subjects (FIG. 11c).

Finally, the best weighted combination able to distinguish familial-CCM1 from familial-CCM3 patients was achieved with only proteins and reached a 100% sensitivity and 82% specificity (FIGS. 11e and f):

$$Canonical Value = - 2.33 * [ CRP ] + 1.64 * [ IL - 6 ] - 5.01 * [ Tsp - 2 ]$$

Lower canonical values (p=0.0018) in familial-CCM1 compared to familial-CCM3 patients (FIG. 11e).

### E. Discussion

Herein, the inventors identify several homologous DE plasma miRNAs in preclinical murine models and CCM patients, with links to genes dysregulated in CCM disease. Additionally, the inventors show that the integration of readily measurable plasma proteins and plasma levels of miRNAs into a combined biomarker can diagnose familial-CCM disease with up to 95% accuracy. To the inventors' knowledge, such a translational approach to biomarker discovery in mouse models and humans, with mechanistic links to the disease transcriptome, validation in an independent cohort of human subjects, and the combination of miRNA and protein levels to optimize biomarker performance have not been previously described in this or any other disease.

This study is the first to show a weighted combination of levels of plasma miRNAs and proteins associated with genotype in a Mendelian disease, based on ML approaches and Bayesian concepts. Two cross-validation methods, which reduced overfitting effects, confirmed that integrating plasma miRNAs and proteins improves the diagnostic association with familial-CCM compared to each circulating compound (e.g., plasma miRNA and protein) alone. This is an important proof of concept for integration of several types of molecules in plasma biomarker development [7]. A homologous biomarker in mice and humans, which can differentiate familial-CCM with germline loss of function mutations can be immediately applied in preclinical studies and early trials, monitoring effectiveness of gene restoration therapeutics, which are currently of great interest. As patients undergo gene therapy, one could monitor their progress and the duration of therapeutic effect as biomarker canonical values shift in comparison to a healthy non-CCM profile. This approach to combined biomarker development is also applicable in other clinical contexts of use, including the prediction of new lesion formation.

In summary, the inventors have shown for the first time in any disease, that homologous DE plasma miRNAs can reflect the germline loss of function mutation in preclinical murine models and patients. They have additionally shown that plasma miRNAs can also distinguish cases who would develop new lesions, and plasma levels of these miRNAs can be assessed for facile diagnostic assays. The inventors describe a novel approach of circulating biomarker development which can be applied in specific clinical contexts of use in familial-CCM and other Mendelian diseases.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.
1. Awad I A, Polster S P. Cavernous angiomas: deconstructing a neurosurgical disease. J Neurosurg. 2019; 131:1-13. https://doi.org/10.3171/2019.3.JNS181724.
2. Akers A, Al-Shahi Salman R, I A A, Dahlem K, Flemming K, Hart B, et al. Synopsis of Guidelines for the Clinical Management of Cerebral Cavernous Malformations: Consensus Recommendations Based on Systematic Literature Review by the Angioma Alliance Scientific Advisory Board Clinical Experts Panel. Neurosurgery. 2017; 80:665-80. https://doi.org/10.1093/neuros/nyx091.
3. Shenkar R, Shi C, Rebeiz T, Stockton R A, McDonald D A, Mikati A G, et al. Exceptional aggressiveness of cerebral cavernous malformation disease associated with PDCD10 mutations. Genet Med. 2015; 17:188-96. https://doi.org/10.1038/gim.2014.97.
4. Polster S P, Cao Y, Carroll T, Flemming K, Girard R, Hanley D, et al. Trial Readiness in Cavernous Angiomas With Symptomatic Hemorrhage (CASH). Neurosurgery. 2019; 84:954-64. https://doi.org/10.1093/neuros/nyyl08.
5. Snellings D A, Hong C C, Ren A A, Lopez-Ramirez M A, Girard R, Srinath A, et al. Cerebral Cavernous Malformation: From Mechanism to Therapy. Circ Res. 2021; 129:195-215. https://doi.org/10.1161/CIRCRESAHA.121.318174.
6. Zhu W, Shen F, Mao L, Zhan L, Kang S, Sun Z, et al. Soluble FLT1 Gene Therapy Alleviates Brain Arteriovenous Malformation Severity. Stroke. 2017; 48:1420-3. https://doi.org/10.1161/STROKEAHA.116.015713.
7. Girard R, Li Y, Stadnik A, Shenkar R, Hobson N, Romanos S, et al. A Roadmap for Developing Plasma Diagnostic and Prognostic Biomarkers of Cerebral Cavernous Angioma With Symptomatic Hemorrhage (CASH). Neurosurgery. 2021; 88:686-97. https://doi.org/10.1093/neuros/nyaa478.
8. Mikati A G, Khanna O, Zhang L, Girard R, Shenkar R, Guo X, et al. Vascular permeability in cerebral cavernous malformations. J Cereb Blood Flow Metab. 2015; 35:1632-9. https://doi.org/10.1038/jcbfm.2015.98.
9. Zeineddine H A, Girard R, Cao Y, Hobson N, Fam M D, Stadnik A, et al. Quantitative susceptibility mapping as a monitoring biomarker in cerebral cavernous malformations with recent hemorrhage. J Magn Reson Imaging. 2018; 47:1133-8. https://doi.org/10.1002/jmri.25831.
10. Girard R, Fam M D, Zeineddine H A, Tan H, Mikati A G, Shi C, et al. Vascular permeability and iron deposition biomarkers in longitudinal follow-up of cerebral cavernous malformations. J Neurosurg. 2017; 127:102-10. https://doi.org/10.3171/2016.5.JNS16687.
11. Huang Z, Ma L, Huang C, Li Q, Nice EC. Proteomic profiling of human plasma for cancer biomarker discovery. Proteomics. 2017;17. https://doi.org/10.1002/pmic.201600240.
12. Loke S Y, Lee A S G. The future of blood-based biomarkers for the early detection of breast cancer. Eur J Cancer. 2018; 92:54-68. https://doi.org/10.1016/j.ejca.2017.12.025.
13. Koskimaki J, Girard R, Li Y, Saadat L, Zeineddine H A, Lightle R, et al. Comprehensive transcriptome analysis of cerebral cavernous malformation across multiple species and genotypes. JCI Insight. 2019; 4. https://doi.org/10.1172/jci.insight.126167.
14. Koskimaki J, Polster S P, Li Y, Romanos S, Srinath A, Zhang D, et al. Common transcriptome, plasma molecules, and imaging signatures in the aging brain and a Mendelian neurovascular disease, cerebral cavernous malformation. Geroscience. 2020; 42:1351-63. https://doi.org/10.1007/s11357-020-00201-4.
15. Lopez-Ramirez M A, Pham A, Girard R, Wyseure T, Hale P, Yamashita A, et al. Cerebral cavernous malformations form an anticoagulant vascular domain in humans and mice. Blood. 2019; 133:193-204. https://doi.org/10.1182/blood-2018-06-856062.
16. Lopez-Ramirez M A, Fonseca G, Zeineddine H A, Girard R, Moore T, Pham A, et al. Thrombospondin1 (TSP1) replacement prevents cerebral cavernous malformations. J Exp Med. 2017; 214:3331-46. https://doi.org/10.1084/jem.20171178.
17. Jenny Zhou H, Qin L, Zhang H, Tang W, Ji W, He Y, et al. Endothelial exocytosis of angiopoietin-2 resulting from CCM3 deficiency contributes to cerebral cavernous malformation. Nat Med. 2016; 22:1033-42. https://doi.org/10.1038/nm.4169.
18. Wustehube J, Bartol A, Liebler S S, Brutsch R, Zhu Y, Felbor U, et al. Cerebral cavernous malformation protein CCM1 inhibits sprouting angiogenesis by activating DELTA-NOTCH signaling. Proc Natl Acad Sci USA. 2010; 107:12640-5. https://doi.org/10.1073/pnas.1000132107.
19. Stockton R A, Shenkar R, Awad I A, Ginsberg M H. Cerebral cavernous malformations proteins inhibit Rho kinase to stabilize vascular integrity. J Exp Med. 2010; 207:881-96. https://doi.org/10.1084/jem.20091258.
20. Wetzel-Strong S E, Weinsheimer S, Nelson J, Pawlikowska L, Clark D, Starr M D, et al. Pilot investigation of circulating angiogenic and inflammatory biomarkers associated with vascular malformations. Orphanet J Rare Dis. 2021; 16:372. https://doi.org/10.1186/s13023-021-02009-7.
21. Koskimaki J, Zhang D, Li Y, Saadat L, Moore T, Lightle R, et al. Transcriptome clarifies mechanisms of lesion genesis versus progression in models of Ccm3 cerebral cavernous malformations. Acta Neuropathol Commun. 2019; 7:132. https://doi.org/10.1186/s40478-019-0789-0.
22. Correia de Sousa M, Gjorgjieva M, Dolicka D, Sobolewski C, Foti M. Deciphering miRNAs' Action through miRNA Int J Editing. Mol Sci. 2019; 20. https://doi.org/10.3390/ijms20246249.
23. Backes C, Meese E, Keller A. Specific miRNA Disease Biomarkers in Blood, Serum and Plasma: Challenges and Prospects. Mol Diagn Ther. 2016; 20:509-18. https://doi.org/10.1007/s40291-016-0221-4.
24. Lugli G, Cohen A M, Bennett D A, Shah R C, Fields C J, Hernandez A G, et al. Plasma Exosomal miRNAs in Persons with and without Alzheimer Disease: Altered Expression and Prospects for Biomarkers. PLOS One. 2015;10: e0139233. https://doi.org/10.1371/journal.pone.0139233.
25. Kar S, Bali K K, Baisantry A, Geffers R, Samii A, Bertalanffy H. Genome-Wide Sequencing Reveals MicroRNAs Downregulated in Cerebral Cavernous Malformations. J Mol Neurosci. 2017; 61:178-88. https://doi.org/10.1007/s12031-017-0880-6.
26. Lyne S B, Girard R, Koskimaki J, Zeineddine H A, Zhang D, Cao Y, et al. Biomarkers of cavernous angioma with symptomatic Insight. 2019; 4. hemorrhage. JCI https://doi.org/10.1172/jci.insight.128577.
27. Wendler A, Wehling M. The translatability of animal models for clinical development: biomarkers and disease models. Curr Opin Pharmacol. 2010; 10:601-6. https://doi.org/10.1016/j.coph.2010.05.009.
28. Tang A T, Choi J P, Kotzin J J, Yang Y, Hong C C, Hobson N, et al. Endothelial TLR4 and the microbiome drive cerebral cavernous malformations. Nature. 2017; 545:305-10. https://doi.org/10.1038/nature22075.
29. Morrison L, Akers A. Cerebral Cavernous Malformation, Familial. In: Adam MP, Ardinger H H, Pagon R A, Wallace S E, Bean L J H, Gripp K W, et al., editors. GeneReviews ((R)). Seattle (WA) 1993.
30. de Champfleur N M, Langlois C, Ankenbrandt W J, Le Bars E, Leroy M A, Duffau H, et al. Magnetic resonance imaging evaluation of cerebral cavernous malformations with susceptibility-weighted imaging. Neurosurgery. 2011; 68:641-7; discussion 7-8. https://doi.org/10.1227/NEU.0b013e31820773cf.
31. Blondal T, Brunetto M R, Cavallone D, Mikkelsen M, Thorsen M, Mang Y, et al. Genome-Wide Comparison of Next-Generation Sequencing and qPCR Platforms for microRNA Profiling in Serum. Methods Mol Biol. 2017; 1580:21-44. https://doi.org/10.1007/978-1-4939-6866-4_3.
32. Decker J T, Hall M S, Blaisdell R B, Schwark K, Jeruss J S, Shea L D. Dynamic microRNA activity identifies therapeutic targets in trastuzumab-resistant HER2 (+) breast cancer. Biotechnol Bioeng. 2018; 115:2613-23. https://doi.org/10.1002/bit.26791.
33. Kim S H, MacIntyre D A, Binkhamis R, Cook J, Sykes L, Bennett P R, et al. Maternal plasma miRNAs as potential biomarkers for detecting risk of small-for-gestational-age births. EBioMedicine. 2020; 62:103145. https://doi.org/10.1016/j.ebiom.2020.103145.
34. Johnson J J, Loeffert A C, Stokes J, Olympia R P, Bramley H, Hicks S D. Association of Salivary MicroRNA Changes With Prolonged Concussion Symptoms. JAMA Pediatr. 2018; 172:65-73. https://doi.org/10.1001/jamapediatrics.2017.3884.
35. Rohart F, Gautier B, Singh A, Le Cao K A. mixOmics: An R package for ‘omics feature selection and multiple data integration. PLOS Comput Biol. 2017;13: e1005752. https://doi.org/10.1371/journal.pcbi.1005752.
36. Dweep H, Gretz N, Sticht C. miRWalk database for miRNA-target interactions. Methods Mol Biol. 2014; 1182:289-305. https://doi.org/10.1007/978-1-4939-1062-5_25.
37. Sticht C, De La Torre C, Parveen A, Gretz N. miRWalk: An online resource for prediction of microRNA binding sites. PLOS One. 2018; 13: e0206239. https://doi.org/10.1371/journal.pone.0206239.
38. Dweep H, Sticht C, Pandey P, Gretz N. miRWalk—database: prediction of possible miRNA binding sites by “walking” the genes of three genomes. J Biomed Inform. 2011; 44:839-47. https://doi.org/10.1016/j.jbi.2011.05.002.
39. Sulakhe D, Balasubramanian S, Xie B, Feng B, Taylor A, Wang S, et al. Lynx: a database and knowledge extraction engine for integrative medicine. Nucleic Acids Res. 2014; 42: D1007-12. https://doi.org/10.1093/nar/gkt1166.
40. Amur S, LaVange L, Zineh I, Buckman-Garner S, Woodcock J. Biomarker Qualification: Toward a Multiple Stakeholder Framework for Biomarker Development, Regulatory Acceptance, and Utilization. Clin Pharmacol Ther. 2015; 98:34-46. https://doi.org/10.1002/cpt.136.
41. Iguchi T, Niino N, Tamai S, Sakurai K, Mori K. Absolute Quantification of Plasma MicroRNA Levels in Cynomolgus Monkeys, Using Quantitative Real-time Reverse Transcription PCR. J Vis Exp. 2018. https://doi.org/10.3791/56850.
42. Johnston S, Gallaher Z, Czaja K. Exogenous reference gene normalization for real-time reverse transcription-polymerase chain reaction analysis under dynamic endogenous transcription. Neural Regen Res. 2012; 7:1064-72. https://doi.org/10.3969/j.issn.1673-5374.2012.14.004.
43. Dakterzada F, Targa A, Benitez I D, Romero-ElKhayat L, de Gonzalo-Calvo D, Torres G, et al. Identification and validation of endogenous control miRNAs in plasma samples for normalization of qPCR data for Alzheimer's disease. Alzheimers Res Ther. 2020; 12:163. https://doi.org/10.1186/s13195-020-00735-x.
44. Andersen C L, Jensen J L, Orntoft T F. Normalization of real-time quantitative reverse transcription-PCR data: a model-based variance estimation approach to identify genes suited for normalization, applied to bladder and colon cancer data sets. Cancer Res. 2004; 64:5245-50. https://doi.org/10.1158/0008-5472.CAN-04-0496.
45. Pfaffl M W. A new mathematical model for relative quantification in real-time RT-PCR. Nucleic Acids Res. 2001;29: e45. https://doi.org/10.1093/nar/29.9.e45.
46. Selst M V, Jolicoeur P. A solution to the effect of sample size on outlier elimination. The quarterly journal of experimental psychology. 1994; 47:631-50.
47. Davies L, Gather U. The identification of multiple outliers. Journal of the American Statistical Association. 1993; 88:782-92.
48. Yuan J S, Reed A, Chen F, Stewart C N, Jr. Statistical analysis of real-time PCR data. BMC Bioinformatics. 2006; 7:85. https://doi.org/10.1186/1471-2105-7-85.
49. Dridi N, Giremus A, Giovannelli J F, Truntzer C, Hadzagic M, Charrier J P, et al. Bayesian inference for biomarker discovery in proteomics: an analytic solution. EURASIP J Bioinform Syst Biol. 2017; 2017:9. https://doi.org/10.1186/s13637-017-0062-4.
50. Girard R, Zeineddine H A, Koskimaki J, Fam M D, Cao Y, Shi C, et al. Plasma Biomarkers of Inflammation and Angiogenesis Predict Cerebral Cavernous Malformation Symptomatic Hemorrhage or Lesional Growth. Circ Res. 2018; 122:1716-21. https://doi.org/10.1161/CIRCRESAHA.118.312680.
51. Girard R, Zeineddine H A, Fam M D, Mayampurath A, Cao Y, Shi C, et al. Plasma Biomarkers of Inflammation Reflect Seizures and Hemorrhagic Activity of Cerebral Cavernous Malformations. Transl Stroke Res. 2018; 9:34-43. https://doi.org/10.1007/s12975-017-0561-3.
52. Arlot S, Celisse A. A survey of cross-validation procedures for model selection. Statistics Surveys. 2010; 4:40-79, 40.
53. Xia J, Broadhurst D I, Wilson M, Wishart D S. Translational biomarker discovery in clinical metabolomics: an introductory tutorial. Metabolomics. 2013; 9:280-99. https://doi.org/10.1007/s11306-012-0482-9.
54. Lien E C, Dibble C C, Toker A. PI3K signaling in cancer: beyond AKT. Curr Opin Cell Biol. 2017; 45:62-71. https://doi.org/10.1016/j.ceb.2017.02.007.
55. Alzahrani A S. PI3K/Akt/mTOR inhibitors in cancer: At the bench and bedside. Semin Cancer Biol. 2019; 59:125-32. https://doi.org/10.1016/j.semcancer.2019.07.009.
56. Chen H, Zhou L, Wu X, Li R, Wen J, Sha J, et al. The PI3K/AKT pathway in the pathogenesis of prostate cancer. Front Biosci (Landmark Ed). 2016; 21:1084-91. https://doi.org/10.2741/4443.
57. Kar S, Samii A, Bertalanffy H. PTEN/PI3K/Akt/VEGF signaling and the cross talk to KRIT1, CCM2, and PDCD10 proteins in cerebral cavernous malformations. Neurosurg Rev. 2015; 38:229-36; discussion 36-7. https://doi.org/10.1007/s10143-014-0597-8.
58. Martinez-Lopez A, Salvador-Rodriguez L, Montero-Vilchez T, Molina-Leyva A, Tercedor-Sanchez J, Arias-Santiago S. Vascular malformations syndromes: an update. Curr Opin Pediatr. 2019; 31:747-53. https://doi.org/10.1097/MOP.0000000000000812.
59. Ren A A, Snellings DA, Su Y S, Hong C C, Castro M, Tang A T, et al. PIK3CA and CCM mutations fuel cavernomas through a cancer-like mechanism. Nature. 2021. https://doi.org/10.1038/s41586-021-03562-8.
60. Hong T, Xiao X, Ren J, Cui B, Zong Y, Zou J, et al. Somatic MAP3K3 and PIK3CA mutations in sporadic cerebral and spinal cord cavernous malformations. Brain. 2021. https://doi.org/10.1093/brain/awab117.
61. Weng J, Yang Y, Song D, Huo R, Li H, Chen Y, et al. Somatic MAP3K3 mutation defines a subclass of cerebral cavernous malformation. Am J Hum Genet. 2021; 108:942-50. https://doi.org/10.1016/j.ajhg.2021.04.005.
62. Lisowska J, Rodel C J, Manet S, Miroshnikova Y A, Boyault C, Planus E, et al. The CCM1-CCM2 complex controls complementary functions of ROCK1 and ROCK2 that are required for endothelial integrity. J Cell Sci. 2018; 131. https://doi.org/10.1242/jcs.216093.
63. McKerracher L, Shenkar R, Abbinanti M, Cao Y, Peiper A, Liao J K, et al. A Brain-Targeted Orally Available ROCK2 Inhibitor Benefits Mild and Aggressive Cavernous Angioma Disease. Transl Stroke Res. 2020; 11:365-76. https://doi.org/10.1007/s12975-019-00725-8.
64. Wei S, Li Y, Polster S P, Weber C R, Awad I A, Shen L. Cerebral Cavernous Malformation Proteins in Barrier Maintenance and Regulation. Int J Mol Sci. 2020; 21. https://doi.org/10.3390/ijms21020675.
65. Polster S P, Stadnik A, Akers A L, Cao Y, Christoforidis G A, Fam M D, et al. Atorvastatin Treatment of Cavernous Angiomas with Symptomatic Hemorrhage Exploratory Proof of Concept (AT CASH EPOC) Trial. Neurosurgery. 2019; 85:843-53. https://doi.org/10.1093/neuros/nyy539.
66. Zhou Z, Tang A T, Wong W Y, Bamezai S, Goddard L M, Shenkar R, et al. Cerebral cavernous malformations arise from endothelial gain of MEKK3-KLF2/4 signalling. Nature. 2016; 532:122-6. https://doi.org/10.1038/nature17178.
67. Lopez-Ramirez M A, Lai C C, Soliman S I, Hale P, Pham A, Estrada E J, et al. Astrocytes propel neurovascular dysfunction during cerebral cavernous malformation lesion formation. J Clin Invest. 2021; 131. https://doi.org/10.1172/JCI139570.
68. Park J H, Lee J Y, Shin D H, Jang K S, Kim H J, Kong G. Loss of Mel-18 induces tumor angiogenesis through enhancing the activity and expression of HIF-1alpha mediated by the PTEN/PI3K/Akt pathway. Oncogene. 2011; 30:4578-89. https://doi.org/10.1038/onc.2011.174.
69. Hong C C, Tang A T, Detter M R, Choi J P, Wang R, Yang X, et al. Cerebral cavernous malformations are driven by ADAMTS5 proteolysis of versican. J Exp Med. 2020; 217. https://doi.org/10.1084/jem.20200140.
70. Liu M, Banerjee R, Rossa C, Jr., D′Silva NJ. RAP1-RAC1 Signaling Has an Important Role in Adhesion and Migration in HNSCC. J Dent Res. 2020; 99:959-68. https://doi.org/10.1177/0022034520917058.
71. Rho S S, Ando K, Fukuhara S. Dynamic Regulation of Vascular Permeability by Vascular Endothelial Cadherin-Mediated Endothelial Cell-Cell Junctions. J Nippon Med Sch. 2017; 84:148-59. https://doi.org/10.1272/jnms.84.148.
72. Li X, Zhang R, Draheim K M, Liu W, Calderwood D A, Boggon T J. Structural basis for small G protein effector interaction of Ras-related protein 1 (Rap1) and adaptor protein Krev interaction trapped 1 (KRIT1). J Biol Chem. 2012; 287:22317-27. https://doi.org/10.1074/jbc.M112.361295.
73. Wu M, Wang G, Tian W, Deng Y, Xu Y. MiRNA-based Therapeutics for Lung Cancer. Curr Pharm Des. 2018; 23:5989-96. https://doi.org/10.2174/1381612823666170714151715.
74. Rupaimoole R, Slack FJ. MicroRNA therapeutics: towards a new era for the management of cancer and other diseases. Nat Rev Drug Discov. 2017; 16:203-22. https://doi.org/10.1038/nrd.2016.246.
75. Burns T C, Li M D, Mehta S, Awad A J, Morgan A A. Mouse models rarely mimic the transcriptome of human neurodegenerative diseases: A systematic bioinformatics-based critique of preclinical models. Eur J Pharmacol. 2015; 759:101-17. https://doi.org/10.1016/j.ejphar.2015.03.021.
76. Mullane K, Williams M. Preclinical Models of Alzheimer's Disease: Relevance and Translational Validity. Curr Protoc Pharmacol. 2019; 84: e57. https://doi.org/10.1002/cpph.57.
77. Shenkar R, Venkatasubramanian P N, Wyrwicz A M, Zhao J C, Shi C, Akers A, et al. Advanced magnetic resonance imaging of cerebral cavernous malformations: part II. Imaging of lesions in murine models. Neurosurgery. 2008; 63:790-7; discussion 7-8. https://doi.org/10.1227/01.NEU.0000315862.24920.49.
78. Zeineddine H A, Girard R, Saadat L, Shen L, Lightle R, Moore T, et al. Phenotypic characterization of murine models of cerebral cavernous malformations. Lab Invest. 2019; 99:319-30. https://doi.org/10.1038/s41374-018-0030-y.
79. Nagy Z B, Bartak B K, Kalmar A, Galamb O, Wichmann B, Dank M, et al. Comparison of Circulating miRNAs Expression Alterations in Matched Tissue and Plasma Samples During Colorectal Cancer Progression. Pathol Oncol Res. 2019; 25:97-105. https://doi.org/10.1007/s12253-017-0308-1.
80. Malczewska A, Kidd M, Matar S, Kos-Kudla B, Modlin I M. A Comprehensive Assessment of the Role of miRNAs as Biomarkers in Gastroenteropancreatic Neuroendocrine Tumors. Neuroendocrinology. 2018; 107:73-90. https://doi.org/10.1159/000487326.

What is claimed:

1. A method for treating cerebral cavernous malformations (CCMs) in a subject, the method comprising administering a therapy to a subject determined to have differential expression of one or more biomarkers selected from the biomarkers listed in Tables S1-S8 and S13 in a biological sample from the subject.

2. A method for treating cerebral cavernous malformations (CCMs) in a subject, the method comprising administering a therapy to a subject determined to have differential expression of one or more biomarkers selected from let-7e-5p, miR-93-5p, miR-20b-5p, miR-128-3p, miR-9-5p, IL-10, Tsp-2, IL-2, and TNFR1.

3. The method of claim 2, wherein the subject has been determined to have to have differential expression of one or more of let-7e-5p, miR-93-5p, miR-20b-5p, miR-128-3p, IL-10, and Tsp-2.

4. The method of claim 3, wherein the subject has been determined to have to have differential expression of let-7e-5p, miR-93-5p, miR-20b-5p, miR-128-3p, IL-10, and Tsp-2.

5. The method of claim 4, wherein the subject has been determined to have a canonical value of greater than 0, wherein the canonical value is equal to −114.9*[let-7e-5p]ᵣ+359.5*[miR-93-5p]ᵣ−118.2*[miR-20b-5p]+88.9*[miR-128-3p]+199.5*[IL-10]−19.7*[Tsp-2]−463.2, wherein [X]ᵣ denotes the relative quantification value for each miRNA biomarker.

6. The method of claim 2, wherein the subject has been determined to have to have differential expression of one or more of miR-9-5p, miR-93-5p, IL-2, TNFR1, and Tsp-2.

7. The method of claim 6, wherein the subject has been determined to have to have differential expression of miR-9-5p, miR-93-5p, IL-2, TNFR1, and Tsp-2.

8. The method of claim 7, wherein the subject has been determined to have a canonical value of less than 15, wherein the canonical value is equal to 2.1*[miR-9-5p]ᵣ−3.0*[miR-93-5p]ᵣ−0.01*[IL-2]+0.1*[TNFR1]+0.5*[Tsp-2], wherein [X]ᵣ denotes the relative quantification value for each miRNA biomarker.

9. The method of any one of claims 1-8, wherein the biomarker is increased relative to a control.

10. The method of any one of claims 1-8, wherein the biomarker is decreased relative to a control.

11. The method of claim 1, wherein the subject has been diagnosed with CCM.

12. The method of claim 1, wherein the subject has not been diagnosed with CCM.

13. The method of any one of claims 1-12, wherein CCM is further defined as familial CCM.

14. The method of claim 13, wherein the familial CCM is further define as familial-CCM1.

15. The method of claim 13, wherein the familial CCM is further define as familial-CCM3.

16. The method of any one of claims 1-16, wherein the subject is being treated with a therapy for CCM.

17. The method of any one of claims 1-16, wherein the sample from the subject comprises a tissue sample, a blood sample, a whole blood sample, a fractionated sample, a plasma sample, a fecal sample, or a urine sample.

18. The method of any one of claims 1-17, wherein the sample from the subject comprises a plasma sample from the subject.

19. The method of any one of claims 1-18, wherein at least the expression level of miR-9-5p was determined.

20. The method of any one of claims 1-19, wherein at least the expression level of miR-93-5p was determined.

21. The method of any one of claims 1-20, wherein at least the expression level of IL-2 was determined.

22. The method of any one of claims 1-21, wherein at least the expression level of TNFR1 was determined.

23. The method of any one of claims 1-22, wherein at least the expression level of Tsp-2 was determined.

24. The method of any one of claims 1-23, wherein at least the expression level of let-7e-5p was determined.

25. The method of any one of claims 1-24, wherein at least the expression level of miR-20b-5p was determined.

26. The method of any one of claims 1-25, wherein at least the expression level of miR-128-3p was determined.

27. The method of any one of claims 1-26, wherein at least the expression level of IL-10 was determined.

28. The method of any of claims 1-27, wherein the expression level of no other biomarker in the sample was determined.

29. The method of any one of claims 1-28, wherein the determined biomarker comprises one or more of let-7e-5p, miR-93-5p, miR-20b-5p, miR-128-3p, and miR-9-5p, and wherein the expression level(s) of the biomarker(s) were determined by evaluating the RNA level of the biomarker.

30. The method of any one of claims 1-29, wherein the biomarker comprises one or more of IL-10, Tsp-2, IL-2, and TNER1, and wherein the expression level(s) of the biomarker(s) were determined by evaluating the protein level of the biomarker.

31. The method of any one of claims 9-30, wherein the control comprises the expression or protein level of the biomarker in samples from subjects identified as low risk or in subjects identified as not having CCM.

32. The method of any one of claims 9-30, wherein the control comprises the expression or protein level of the biomarker in samples from subjects identified as high risk or in subjects identified as having CCM.

33. The method of any one of claims 1-32, wherein the therapy comprises a gene therapy, a B-cell immunomodulation therapy, surgical excision of a CCM lesion, or combinations thereof.

34. A method for evaluating a subject comprising measuring the level of expression of one or more biomarkers selected from the biomarkers listed in Tables S1-S8 and S13 in a biological sample from the subject.

35. A method for evaluating a subject comprising measuring the level of expression of one or more of let-7e-5p, miR-93-5p, miR-20b-5p, miR-128-3p, miR-9-5p, IL-10, Tsp-2, IL-2, and TNFR1 in a sample from the subject.

36. The method of claim 35, wherein the expression levels of one or more of let-7e-5p, miR-93-5p, miR-20b-5p, miR-128-3p, IL-10, and Tsp-2 are evaluated in the sample.

37. The method of claim 36, wherein the expression levels of let-7e-5p, miR-93-5p, miR-20b-5p, miR-128-3p, IL-10, and Tsp-2 are evaluated in the sample.

38. The method of claim 37, wherein the method further comprises determining a canonical value; wherein the canonical value was determined to be greater than 0, wherein the canonical value is equal to −114.9*[let-7e-5p]ᵣ+359.5*[miR-93-5p]ᵣ−118.2*[miR-20b-5p]ᵣ+88.9*[miR-128-3p]ᵣ+199.5*[IL-10]−19.7*[Tsp-2]−463.2, wherein [X]ᵣ denotes the relative quantification value for each miRNA biomarker.

39. The method of claim 35, wherein the expression levels of one or more of miR-9-5p, miR-93-5p, IL-2, TNFR1, and Tsp-2 are evaluated in the sample.

40. The method of claim 39, wherein the expression levels of miR-9-5p, miR-93-5p, IL-2, TNFR1, and Tsp-2 are evaluated in the sample.

41. The method of claim 40, wherein the method further comprises determining a canonical value; wherein the canonical value was determined to be less than 15, wherein the canonical value is equal to 2.1*[miR-9-5p]ᵣ−3.0*[miR-93-5p]ᵣ−0.01*[IL-2]+0.1*[TNFR1]+0.5*[Tsp-2], wherein [X]ᵣ denotes the relative quantification value for each miRNA biomarker.

42. The method of any one of claims 34-41, wherein the biomarker is increased relative to a control.

43. The method of any one of claims 34-41, wherein the biomarker is decreased relative to a control.

44. The method of any one of claims 34-44, wherein the subject has been diagnosed with CCM.

45. The method of any one of claims 34-44, wherein the subject has not been diagnosed with CCM.

46. The method of any one of claims 34-45, wherein CCM is further defined as familial CCM.

47. The method of claim 46, wherein the familial CCM is further define as familial-CCM1.

48. The method of claim 46, wherein the familial CCM is further define as familial-CCM3.

49. The method of any one of claims 34-48, wherein the subject is being treated with a therapy for CCM.

50. The method of any one of claims 34-49, wherein the sample from the subject comprises a tissue sample, a blood sample, a whole blood sample, a fractionated sample, a plasma sample, a fecal sample, or a urine sample.

51. The method of any one of claims 34-50, wherein the sample from the subject comprises a plasma sample from the subject.

52. The method of any one of claims 34-51, wherein at least the expression level of miR-9-5p is evaluated.

53. The method of any one of claims 34-52, wherein at least the expression level of miR-93-5p is evaluated.

54. The method of any one of claims 34-53, wherein at least the expression level of IL-2 is evaluated.

55. The method of any one of claims 34-54, wherein at least the expression level of TNFR1 is evaluated.

56. The method of any one of claims 34-55, wherein at least the expression level of Tsp-2 is evaluated.

57. The method of any one of claims 34-56, wherein at least the expression level of let-7e-5p is evaluated.

58. The method of any one of claims 34-57, wherein at least the expression level of miR-20b-5p is evaluated.

59. The method of any one of claims 34-58, wherein at least the expression level of miR-128-3p is evaluated.

60. The method of any one of claims 34-59, wherein at least the expression level of IL-10 is evaluated.

61. The method of any of claims 34-60, wherein the expression level of no other biomarker in the biological sample are evaluated.

62. The method of any one of claims 34-61, wherein the biomarker comprises one or more of let-7e-5p, miR-93-5p, miR-20b-5p, miR-128-3p, and miR-9-5p, and wherein the expression level(s) of the biomarker(s) were measured by evaluating the RNA level of the biomarker.

63. The method of any one of claims 34-62, wherein the biomarker comprises one or more of IL-10, Tsp-2, IL-2, and TNER1, and wherein the expression level(s) of the biomarker(s) were measured by evaluating the protein level of the biomarker.

64. The method of any one of claims 42-63, wherein the control comprises the expression or protein level of the biomarker in samples from subjects identified as low risk or in subjects identified as not having CCM.

65. The method of any one of claims 42-63, wherein the control comprises the expression or protein level of the biomarker in samples from subjects identified as high risk or in subjects identified as having CCM.

66. The method of any one of claims 34-65, wherein the method further comprises treating the subject with a CCM therapy.

67. The method of claim 66, wherein the therapy comprises a gene therapy, a B-cell immunomodulation therapy, surgical excision of a CCM lesion, or combinations thereof.

68. A method of prognosing, diagnosing, and/or determining the efficacy of a therapy in a subject, the method comprising:
a) measuring the level of expression of one or more biomarkers listed in Tables S1-S8 and S13 in a sample from the subject;
b) i) comparing the level(s) of expression of the one or more biomarkers to a control sample(s) or control level(s) of expression; or ii) calculating a canonical value; and,
c) prognosing, diagnosing, and/or determining the efficacy of a therapy based on the levels of measured expression and/or the canonical value.

69. A method of prognosing, diagnosing, and/or determining the efficacy of a therapy in a subject, the method comprising:
a) measuring the level of expression of one or more biomarkers selected from let-7e-5p, miR-93-5p, miR-20b-5p, miR-128-3p, miR-9-5p, IL-10, Tsp-2, IL-2, and TNFR1 in a sample from the subject;
b) i) comparing the level(s) of expression of the one or more biomarkers to a control sample(s) or control level(s) of expression; and/or ii) calculating a canonical value; and,
c) prognosing, diagnosing, and/or determining the efficacy of a therapy based on the levels of measured expression and/or the canonical value.

70. The method of claim 69, wherein the expression levels of one or more of let-7e-5p, miR-93-5p, miR-20b-5p, miR-128-3p, IL-10, and Tsp-2 are measured in the sample.

71. The method of claim 70, wherein the expression levels of let-7e-5p, miR-93-5p, miR-20b-5p, miR-128-3p, IL-10, and Tsp-2 are measured in the sample.

72. The method of claim 71, wherein the method comprises determining a canonical value;
wherein the canonical value is equal to −114.9*[let-7e-5p]ᵣ+359.5*[miR-93-5p]ᵣ−118.2*[miR-20b-5p]ᵣ+88.9*[miR-128-3p]ᵣ+199.5*[IL-10]−19.7*[Tsp-2]−463.2, wherein [X]ᵣ denotes the relative quantification value for each miRNA biomarker.

73. The method of claim 72, wherein the subject is diagnosed as having CCM or prognosed as high risk when the canonical value is greater than 0.

74. The method of claim 72 or 73, wherein the efficacy of the therapy is determined to be ineffective when the canonical value is greater than 0.

75. The method of claim 73 or 74, wherein the method further comprises treating the subject with a CCM therapy.

76. The method of claim 75, wherein the therapy comprises a gene therapy, a B-cell immunomodulation therapy, surgical excision of a CCM lesion, or combinations thereof.

77. The method of claim 69, wherein the expression levels of one or more of miR-9-5p, miR-93-5p, IL-2, TNFR1, and Tsp-2 are measured in the sample.

78. The method of claim 77, wherein the expression levels of miR-9-5p, miR-93-5p, IL-2, TNFR1, and Tsp-2 are evaluated in the sample.

79. The method of claim 78, wherein the method further comprises determining a canonical value; wherein the canonical value is equal to 2.1*[miR-9-5p]ᵣ−3.0*[miR-93-5p]ᵣ−0.01*[IL-2]+0.1*[TNFR1]+0.5*[Tsp-2], wherein [X]ᵣ denotes the relative quantification value for each miRNA biomarker.

80. The method of claim 79, wherein the subject is diagnosed as having CCM or prognosed as high risk when the canonical value is less than 15.

81. The method of claim 79 or 80, wherein the efficacy of the therapy is determined to be ineffective when the canonical value is less than 15.

82. The method of claim 80 or 81, wherein the method further comprises treating the subject with a CCM therapy.

83. The method of claim 82, wherein the therapy comprises a gene therapy, a B-cell immunomodulation therapy, surgical excision of a CCM lesion, or combinations thereof.

84. The method of any one of claims 68-83, wherein the subject has been diagnosed with CCM.

85. The method of any one of claims 68-83, wherein the subject has not been diagnosed with CCM.

86. The method of any one of claims 68-85, wherein CCM is further defined as familial CCM.

87. The method of claim 86, wherein the familial CCM is further define as familial-CCM1.

88. The method of claim 86, wherein the familial CCM is further define as familial-CCM3.

89. The method of any one of claims 68-88, wherein the subject is being treated with a therapy for CCM.

90. The method of any one of claims 68-89, wherein the sample from the subject comprises a tissue sample, a blood sample, a whole blood sample, a fractionated sample, a plasma sample, a fecal sample, or a urine sample.

91. The method of any one of claims 68-90, wherein the sample from the subject comprises a plasma sample from the subject.

92. The method of any one of claims 68-91, wherein at least the expression level of miR-9-5p is measured.

93. The method of any one of claims 68-92, wherein at least the expression level of miR-93-5p is measured.

94. The method of any one of claims 68-93, wherein at least the expression level of IL-2 is measured.

95. The method of any one of claims 68-94, wherein at least the expression level of TNFR1 is measured.

96. The method of any one of claims 68-95, wherein at least the expression level of Tsp-2 is measured.

97. The method of any one of claims 68-96, wherein at least the expression level of let-7e-5p is measured.

98. The method of any one of claims 68-97, wherein at least the expression level of miR-20b-5p is measured.

99. The method of any one of claims 68-98, wherein at least the expression level of miR-128-3p is measured.

100. The method of any one of claims 68-99, wherein at least the expression level of IL-10 is measured.

101. The method of any of claims 68-100, wherein the expression level of no other biomarker in the biological sample are measured.

102. The method of any one of claims 68-101, wherein the biomarker is increased relative to a control.

103. The method of any one of claims 68-102, wherein the biomarker is decreased relative to a control.

104. The method of any one of claims 68-103, wherein the biomarker comprises one or more of let-7e-5p, miR-93-5p, miR-20b-5p, miR-128-3p, and miR-9-5p, and wherein the expression level(s) of the biomarker(s) were measured by evaluating the RNA level of the biomarker.

105. The method of any one of claims 68-104, wherein the biomarker comprises one or more of IL-10, Tsp-2, IL-2, and TNER1, and wherein the expression level(s) of the biomarker(s) were measured by evaluating the protein level of the biomarker.

106. The method of any one of claims 68-105, wherein the control comprises the expression or protein level of the biomarker in samples from subjects identified as low risk or in subjects identified as not having CCM.

107. The method of any one of claims 68-105, wherein the control comprises the expression or protein level of the biomarker in samples from subjects identified as high risk or in subjects identified as having CCM.

108. A kit comprising 1, 2, 3, 4, 5, 6, 7, 8, 9, 10, 11, 12, 13, 14, or 15 detection agents for determining expression levels of biomarkers for CCM, wherein the biomarkers comprise one or more listed in Tables S1-S8 and S13.

109. A kit comprising 1, 2, 3, 4, 5, 6, 7, 8, 9, 10, 11, 12, 13, 14, or 15 detection agents for determining expression levels of biomarkers for CCM, wherein the biomarkers comprise one or more of let-7e-5p, miR-93-5p, miR-20b-5p, miR-128-3p, miR-9-5p, IL-10, Tsp-2, IL-2, and TNFR1.

110. The kit of claim 108 or 109, wherein the kit further comprises one or more negative or positive control samples and/or control detection agents.

111. The kit of any one of claims 108-110, wherein the kit further comprises instructions for use.

112. The kit of any one of claims 108-110, wherein the CCM biomarkers comprise one or more of let-7e-5p, miR-93-5p, miR-20b-5p, miR-128-3p, IL-10, and Tsp-2.

113. The kit of claim 112, wherein the CCM biomarkers consist of let-7e-5p, miR-93-5p, miR-20b-5p, miR-128-3p, IL-10, and Tsp-2.

114. The kit of claim 112, wherein the CCM biomarkers comprise or consist of let-7e-5p, miR-93-5p, miR-20b-5p, miR-128-3p, IL-10, Tsp-2 and/or one or more controls.

115. The kit of any one of claims 111-114, wherein the instructions comprises evaluating a canonical value, wherein the canonical value is equal to −114.9*[let-7e-5p]ᵣ+359.5*[miR-93-5p]ᵣ−118.2*[miR-20b-5p]ᵣ+88.9*[miR-128-3p]ᵣ+199.5*[IL-10]−19.7*[Tsp-2]−463.2, wherein [X]ᵣ denotes the relative quantification value for each miRNA biomarker.

116. The kit of any one of claims 108-109, wherein the CCM biomarkers comprise one or more of miR-9-5p, miR-93-5p, IL-2, TNFR1, and Tsp-2.

117. The kit of claim 116, wherein the CCM biomarkers consist of miR-9-5p, miR-93-5p, IL-2, TNFR1, and Tsp-2.

118. The kit of claim 116, wherein the CCM biomarkers comprise or consist of miR-9-5p, miR-93-5p, IL-2, TNFR1, Tsp-2, and/or one or more controls.

119. The kit of any one of claims 111-118, wherein the instructions comprises evaluating a canonical value, wherein the canonical value is equal to 2.1*[miR-9-5p]ᵣ−3.0*[miR-93-5p]ᵣ −0.01*[IL-2]+0.1*[TNFR1]+0.5*[Tsp-2], wherein [X]ᵣ denotes the relative quantification value for each miRNA biomarker.

120. The kit of any one of claims 109-119, wherein the kit comprises reagents for isolation and/or amplification of biomarkers from a biological sample.

121. The kit of claim 120, wherein the biological sample comprises a tissue sample, a blood sample, a whole blood sample, a fractionated sample, a plasma sample, a fecal sample, or a urine sample.

122. The kit of claim 121, wherein the biological sample comprises a plasma sample.

123. The kit of any one of claims 120-122, wherein the biological sample is from a subject.

124. The kit of claim 123, wherein the subject has been diagnosed with CCM.

125. The kit of claim 123, wherein the subject has not been diagnosed with CCM.

126. The kit of claim 124 or 125, wherein CCM is further defined as familial CCM.

127. The kit of claim 126, wherein the familial CCM is further define as familial-CCM1.

128. The kit of claim 126, wherein the familial CCM is further define as familial-CCM3.

129. The kit of any one of claims 123-128, wherein the subject is being treated with a therapy for CCM.

130. The kit of claim 129, wherein the therapy comprises a gene therapy, a B-cell immunomodulation therapy, surgical excision of a CCM lesion, or combinations thereof.

131. A method for treating cerebral cavernous malformations (CCMs) in a subject, the method comprising administering one or more miRNA modulators, wherein the miRNA comprises let-7e-5p, miR-93-5p, miR-20b-5p, miR-128-3p, and/or miR-9-5p.

132. The method of claim 131, wherein the modulator is an agomer of the miRNA.

133. The method of claim 131, wherein the modulator is an antagomir of the miRNA.

134. The method of any one of claims 131-133, wherein the modulator is linked to a targeting moiety.

135. The method of claim 134, wherein the targeting moiety is an aptamer.

136. The method of claim 134 or 135, wherein the targeting moiety delivers the modulator to a specific cell type or tissue.

137. The method of any one of claims 131-136, wherein the modulator comprise a modified oligonucleotide.

138. The method of claim 137, wherein the modified oligonucleotide comprises a locked nucleotide, ethylene bridged nucleotide, a peptide nucleic acid, or a 5’ (E)-vinyl-phosphonate (VP) modification.

139. The method of any one of claims 131-138, wherein the modulator is administered by systemic, intravenous, intramuscular, or parenteral administration.

140. The method of any one of claims 131-139, wherein the subject has been diagnosed with CCM.

141. The method of any one of claims 131-139, wherein the subject has not been diagnosed with CCM.

142. The method of any one of claims 131-141, wherein CCM is further defined as familial CCM.

143. The method of claim 142, wherein the familial CCM is further define as familial-CCM1.

144. The method of claim 13, wherein the familial CCM is further define as familial-CCM3.

145. The method of any one of claims 131-144, wherein the subject is being treated with a therapy for CCM.

146. The method of claim 145, wherein the therapy comprises a gene therapy, a B-cell immunomodulation therapy, surgical excision of a CCM lesion, or combinations thereof.

147. The method of any one of claims 131-146, wherein the subject has been determined to have differential expression of one or more biomarkers selected from the biomarkers listed in Tables S1-S8 and S13 in a biological sample from the subject.

148. The method of any one of claims 131-147, wherein the subject has been determined to have differential expression of one or more biomarkers selected from let-7e-5p, miR-93-5p, miR-20b-5p, miR-128-3p, miR-9-5p, IL-10, Tsp-2, IL-2, and TNER1.
